SEQUENCE LISTING (42 sequences)

SEQ ID NO: 1 - DNA, 60 bp, synthetic construct
  source 1..60: mol_type = other DNA
atgtctcgctccgtggccttagctgtgctcgcgctactctctctttctggcctcgaggct

SEQ ID NO: 2 - protein, 20 aa, synthetic construct
MSRSVALAVLALLSLSGLEA

SEQ ID NO: 3 - DNA, 297 bp, synthetic construct
  source 1..297: mol_type = other DNA
atccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgagttcacccccactgaaaaagatgagtatgcctgccgtgtgaaccatgtgactttgtcacagcccaagatagttaagtgggatcgcgacatg

SEQ ID NO: 4 - protein, 99 aa, synthetic construct [sequence omitted]

SEQ ID NO: 5 - DNA, 27 bp, synthetic construct
  source 1..27: mol_type = other DNA
gttatggctccgcggactttaatttta

SEQ ID NO: 6 - protein, 9 aa, synthetic construct
VMAPRTLIL

SEQ ID NO: 7 - DNA, 45 bp, synthetic construct
  source 1..45: mol_type = other DNA
ggtggtggcggatccggtggtggcggttctggtggtggcggctcc

SEQ ID NO: 8 - protein, 15 aa, synthetic construct
GGGGSGGGGSGGGGS

SEQ ID NO: 9 - DNA, 60 bp, synthetic construct
  source 1..60: mol_type = other DNA
ggtggtggcggttctggtggtggcggtagtggcggcggaggaagcggtggtggcggttcc

SEQ ID NO: 10 - protein, 20 aa, synthetic construct
GGGGSGGGGSGGGGSGGGGS

SEQ ID NO: 11 - DNA, 1011 bp, synthetic construct
  source 1..1011: mol_type = other DNA
ggatctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggctctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 12 - protein, 337 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 13 - DNA, 1521 bp, synthetic construct
  source 1..1521: mol_type = other DNA
actagtgccaccatgtctcgctccgtggccttagctgtgctcgcgctactctctctttctggcctcgaggctgttatggctccgcggactttaattttaggtggtggcggatccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgagttcacccccactgaaaaagatgagtatgcctgccgtgtgaaccatgtgactttgtcacagcccaagatagttaagtgggatcgcgacatgggtggtggcggttctggtggtggcggtagtggcggcggaggaagcggtggtggcggttccggatctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggctctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaagaattc

SEQ ID NO: 14 - DNA, 27 bp, synthetic construct
  source 1..27: mol_type = other DNA
aaaggacctcctgccgctcttacactt

SEQ ID NO: 15 - protein, 9 aa, synthetic construct
KGPPAALTL

SEQ ID NO: 16 - DNA, 60 bp, synthetic construct; identical to SEQ ID NO: 1
  source 1..60: mol_type = other DNA

SEQ ID NO: 17 - protein, 20 aa, synthetic construct
MSRSVALAVLALLSLSGLEA

SEQ ID NO: 18 - DNA, 297 bp, synthetic construct; identical to SEQ ID NO: 3
  source 1..297: mol_type = other DNA

SEQ ID NO: 19 - protein, 99 aa, synthetic construct [sequence omitted]

SEQ ID NO: 20 - DNA, 945 bp, synthetic construct
  source 1..945: mol_type = other DNA
ggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattga

SEQ ID NO: 21 - protein, 314 aa, synthetic construct
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 22 - DNA, 1452 bp, synthetic construct
  source 1..1452: mol_type = other DNA
actagtgccaccatgtctcgctccgtggccttagctgtgctcgcgctactctctctttctggcctcgaggctaaaggacctcctgccgctcttacacttggtggtggcggatccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgagttcacccccactgaaaaagatgagtatgcctgccgtgtgaaccatgtgactttgtcacagcccaagatagttaagtgggatcgcgacatgggtggtggcggttctggtggtggcggtagtggcggcggaggaagcggtggtggcggttccggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgagaattc

SEQ ID NO: 23 - DNA, 1074 bp, synthetic construct
  source 1..1074: mol_type = other DNA
atggtagatggaaccctccttttactcctctcggaggccctggcccttacccagacctgggcgggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 24 - protein, 358 aa, synthetic construct
MVDGTLLLLLSEALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 25 - DNA, 354 bp, synthetic construct
  source 1..354: mol_type = other DNA
tctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatg

SEQ ID NO: 26 - protein, 118 aa, synthetic construct
SRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 27 - protein, 18 aa, synthetic construct
GGGGSSGGGGSSGGGGSS

SEQ ID NO: 28 - DNA, 1485 bp, synthetic construct
  source 1..1485: mol_type = other DNA
atggtagatggaaccctccttttactcctctcggaggccctggcccttacccagacctgggcgggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgggtggtggtggtagtagtggtggtggtggtagtagtggtggtggtggtagtagttctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaa

SEQ ID NO: 29 - DNA, 357 bp, synthetic construct
  source 1..357: mol_type = other DNA
atgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgagttcacccccactgaaaaagatgagtatgcctgccgtgtgaaccatgtgactttgtcacagcccaagatagttaagtgggatcgagacatg

SEQ ID NO: 30 - protein, 119 aa, synthetic construct
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 31 - DNA, 1029 bp, synthetic construct
  source 1..1029: mol_type = other DNA
aagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattga

SEQ ID NO: 32 - protein, 342 aa, synthetic construct
KTPRMVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 33 - DNA, 1437 bp, synthetic construct
  source 1..1437: mol_type = other DNA
gccaccatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgagttcacccccactgaaaaagatgagtatgcctgccgtgtgaaccatgtgactttgtcacagcccaagatagttaagtgggatcgagacatgggtggtggtggtagtggtggtggtggtagtggtggtggtggtagtaagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattga

SEQ ID NO: 34 - DNA, 11690 bp, synthetic construct
  source 1..11690: mol_type = other DNA
agtcctgcaggtttaaacgaattcgccctttgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggcaagcttctgacctcttctcttcctcccacagggcctcgagagatctggcagcggagagggcagaggaagtcttctaacatgcggtgacgtggaggagaatcccggccctaggctcgagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgtcgacatgtctcgctccgtggccttagctgtgctcgcgctactctctctttctggcctcgaggctgttatggctccgcggactttaattttaggtggtggcggatccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgagttcacccccactgaaaaagatgagtatgcctgccgtgtgaaccatgtgactttgtcacagcccaagatagttaagtgggatcgcgacatgggtggtggcggttctggtggtggcggtagtggcggcggaggaagcggtggtggcggttccggatctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggctctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaaacgcgtgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccaggcgcgtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatcgacagtactaagctttactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctctaagggcgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatgcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgaatactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcagaattaaccctcactaaagggactagct

SEQ ID NO: 35 - DNA, 11621 bp, synthetic construct
  source 1..11621: mol_type = other DNA
agtcctgcaggtttaaacgaattcgccctttgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggcaagcttctgacctcttctcttcctcccacagggcctcgagagatctggcagcggagagggcagaggaagtcttctaacatgcggtgacgtggaggagaatcccggccctaggctcgagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgtcgacatgtctcgctccgtggccttagctgtgctcgcgctactctctctttctggcctcgaggctaaaggacctcctgccgctcttacacttggtggtggcggatccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgagttcacccccactgaaaaagatgagtatgcctgccgtgtgaaccatgtgactttgtcacagcccaagatagttaagtgggatcgcgacatgggtggtggcggttctggtggtggcggtagtggcggcggaggaagcggtggtggcggttccggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaacgcgtgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccaggcgcgtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatcgacagtactaagctttactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctctaagggcgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatgcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgaatactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcagaattaaccctcactaaagggactagct

SEQ ID NO: 36 - DNA, 309 bp, synthetic construct
  source 1..309: mol_type = other DNA
atgactgccatggaggagtcacagtcggatatcagcctcaagagagcgctgcccacctgcacaagcgcctctcccccgcaaaagaaaaaaccacttgatggagagtatttcaccctcaagatccgcgggcgtaaacgcttcgagatgttccgggagctgaatgaggccttagagttaaaggatgcccatgctacagaggagtctggagacagcagggctcactccagctacctgaagaccaagaagggccagtctacttcccgccataaaaaaacaatggtcaagaaagtggggcctgactcagactga

SEQ ID NO: 37 - DNA, 20 bp, synthetic construct
  source 1..20: mol_type = other DNA
cgcgagcacagctaaggcca

SEQ ID NO: 38 - DNA, 21 bp, synthetic construct
  source 1..21: mol_type = other DNA
tgtgaaccatgtgactttgtc

SEQ ID NO: 39 - DNA, 42 bp, synthetic construct
  source 1..42: mol_type = other DNA
atgtctcgctccgtggccttagctgtgctcgcgctactctct

SEQ ID NO: 40 - DNA, 22 bp, synthetic construct
  source 1..22: mol_type = other DNA
agctgtgctcgcgctactctct

SEQ ID NO: 41 - DNA, 41 bp, synthetic construct
  source 1..41: mol_type = other DNA
atgtctcgctccgtggcttagctgtgctcgcgctactctct

SEQ ID NO: 42 - DNA, 32 bp, synthetic construct
  source 1..32: mol_type = other DNA
atgtctcgctccgtgggctcgcgctactctct

(19) United States
(12) Patent Application Publication
(10) Pub. No.: US 2024/0425823 A1
(43) Pub. Date: Dec. 26, 2024

(54) UNIVERSAL STEM CELL AND USES THEREOF

(71) Applicant: THE PENN STATE RESEARCH FOUNDATION, University Park, PA (US)

(72) Inventors: Xiaojun LIAN, University Park, PA (US); Lauren RANDOLPH, University Park, PA (US)

(21) Appl. No.: 18/705,343

(22) PCT Filed: Oct. 27, 2022
(86) PCT No.: PCT/US2022/078775

§ 371 Date: Apr. 26, 2024

Related U.S. Application Data
(60) Provisional application No. 63/272,322, filed on Oct. 27, 2021.

(51) Int. Cl. C12N 5/074; A61K 35/34; C07K 14/74; C07K 14/82; C12N 5/077; C12N 9/22; C12N 15/11

(52) U.S. Cl. CPC C12N 5/0696; A61K 35/34; C07K 14/70539; C07K 14/82; C12N 5/0657; C12N 9/22; C12N 15/11; C07K 2319/02; C12N 2310/20; C12N 2510/00

(57) ABSTRACT

Disclosed herein are recombinant poly nucleotides and universal stem cells comprising said recombinant polynucleotides. Also disclosed herein are method for treating cardiac disorders using the universal stem cells comprising said recombinant polynucleotides.

### CROSS-REFERENCE TO RELATED APPLICATIONS

This application claims the priority benefit of U.S. Provisional Application No. 63/272,322, filed Oct. 27, 2021, which is incorporated herein by reference in its entirety.

### STATEMENT REGARDING FEDERALLY SPONSORED RESEARCH OR DEVELOPMENT

This invention was made with Government Support under Grant NO: R21EB026035 awarded by the National Institutes of Health. The Government has certain rights in the invention.

### BACKGROUND

Human pluripotent stem cells (hPSCs), both embryonic and induced (iPSCs), offer unlimited self-renewal and capacity to differentiate into any desired therapeutic cell type, making these cells an ideal candidate for a universal donor cell line. However, transplanted cells will be rejected by the recipient's immune cells. What are needed are compositions and methods to reduce rejection of stem cells by the recipient's immune cells.

### SUMMARY

Disclosed herein are universal stem cells and methods of making the cells. In some examples, genome editing tools are used to specifically knock out beta-2-Microglobulin (B2M) gene to prevent T cell-mediated lysis towards the transplanted cells. Furthermore, a recombinant polynucleotide that comprises a polynucleotide sequence encoding a HLA-E or a HLA-G polypeptide is introduced into the universal stem cells to prevent natural killer (NK) cell-mediated lysis. These modified stem cells become universal donor stem cells and are not rejected by recipient's immune cells.

The details of one or more embodiments of the invention are set forth in the accompanying drawings and the description below. Other features, objects, and advantages of the invention will be apparent from the description and drawings, and from the claims.

In some aspects, disclosed herein is a recombinant polynucleotide comprising a first polynucleotide sequence encoding a B2M signaling peptide or a fragment thereof; a second polynucleotide sequence encoding a nonamer or a fragment thereof; a third polynucleotide sequence encoding a B2M polypeptide that lacks signaling peptide; and a fourth polynucleotide sequence encoding a human leukocyte antigen (HLA)-E polypeptide or a HLA-G polypeptide or a fragment thereof.

In some embodiments, the fourth polynucleotide sequence encodes a HLA-G polypeptide or a fragment thereof. In some embodiments, the fourth polynucleotide sequence comprises a sequence at least about 80% identity to SEQ ID NO: 20 or a fragment thereof. In some embodiments, the second polynucleotide sequence encoding the nonamer comprises a sequence at least about 80% identity to SEQ ID NO: 14 or a fragment thereof.

In some embodiments, the fourth polynucleotide sequence encodes a HLA-E polypeptide or a fragment thereof. In some embodiments, the fourth polynucleotide sequence comprises a sequence at least about 80% identity to SEQ ID NO: 11 or a fragment thereof. In some embodiments, the nonamer comprises a signal peptide sequence of a class I HLA (e.g., HLA-C). In some embodiments, the second polynucleotide sequence encoding the nonamer is at least about 80% identity to SEQ ID NO: 5 or a fragment thereof.

In some embodiments, the first nucleotide is at least 80% identity to SEQ ID NO: 1 or 16 or a fragment thereof.

In some embodiments, the third nucleotide is at least 80% identity to SEQ ID NO: 3 or 18 or a fragment thereof.

In some embodiments, the recombinant polypeptide disclosed herein comprises a sequence at least 80% identity to SEQ ID NO: 13, 22 or a fragment thereof.

Also disclosed herein is a vector comprising the recombinant polynucleotide disclosed herein. The vector can be an AAVSI locus targeting vector. In some examples, the vector comprises a sequence at least about 80% identity to SEQ ID NO: 34 or 35 or a fragment thereof.

Also disclosed herein is a universal stem cell comprising the recombinant polynucleotide disclosed herein that encodes a HLA-E or a HLA-G polypeptide. The universal stem cell can further comprise a polynucleotide encoding a p53 dominant-negative (p53 DD) polypeptide. In some examples, the universal stem comprising a deletion in a B2M host gene or a fragment thereof. In some examples, the universal stem comprising a deletion in a B2M host gene or a fragment thereof.

Also disclosed herein is a method of making a universal stem cell, comprising transducing a recombinant polynucleotide into the universal stem cell of any preceding aspect, and culturing the transduced universal stem cell, wherein the recombinant polynucleotide comprises a first polynucleotide sequence encoding a B2M signaling peptide or a fragment thereof; a second polynucleotide sequence encoding a nonamer or a fragment thereof; a third polynucleotide sequence encoding a B2M polypeptide that lacks signaling peptide; and a fourth polynucleotide sequence encoding a HLA-E or HLA-G polypeptide or a fragment thereof.

Also disclosed herein is a method making a cardiomyocyte, comprising making a universal stem cell of any preceding aspect by any of the preceding aspects disclosed herein; culturing the universal stem cell for about 12 days in a cell culture media to differentiate the universal stem cell into a cardiomyocyte; and harvesting the differentiated cardiomyocyte.

Also disclosed herein is a method of treating a cardiac disorder in a subject in need, comprising making a universal stem cell by the method disclosed herein; culturing the universal stem cell for about 12 days in a cell culture media to differentiate the universal stem cell into a cardiomyocyte; and transplanting the cardiomyocyte into the subject.

### DESCRIPTION OF DRAWINGS

FIG. 1A, FIG. 1B, and FIG. 1C show design strategy for universal donor hPSCs. FIG. 1A shows schematic illustrating the structure of class I HLA molecules. FIG. 1B shows relative expression for MHC molecules from bulk RNA sequencing analysis of day 60 hPSC derived CMs. FIG. 1C shows schematic of the two-part universal donor stem cell design strategy.

FIG. 2A, FIG. 2B, FIG. 2C, FIG. 2D, FIG. 2E, FIG. 2F, FIG. 2G, FIG. 2H, FIG. 2I, and FIG. 2J show generation of B2M KO hPSCs. FIG. 2A shows schematic illustrating the location sin the B2M gene where the sgRNAs were targeted. FIG. 2B shows experimental schematic for the generation of a B2M KO with HEK 293 cells. FIG. 2C shows brightfield and GFP images of HEK 293 cells in each condition taken 24 hours after transfection. Scale bars are 100 μm. FIG. 2D shows flow cytometry analysis of B2M expression for HEK 293 cells one week after transfection. FIG. 2E shows experimental schematic for the generation of B2M KO hPSCs. FIG. 2F shows brightfield and GFP images of B2M KO cells transfected with p53DD (bottom) and without (top) 24 and 48 hours after nucleofection. Scale bars are 100 μm. FIG. 2G shows flow cytometry quantification of the GFP positive percentage of p53DD treated cells 48 hours after nucleofection. FIGS. 2H-2I show flow cytometry analysis of B2M expression for cells treated with p53DD one week after nucleofection (FIG. 2H) and for WT and selected single cell derived clones for H9 and 6-9-9 (FIG. 2I). FIG. 2J shows Sanger sequencing of WT and KO alleles. Green indicates start codon. Bolded blue indicates sgRNA sequence. Underline indicates the protospacer adjacent motif. Red colons indicate deleted base pairs. The sequences in FIG. 2 are SEQ ID NOs: 39-42.

FIG. 3A, FIG. 3B, FIG. 3C, FIG. 3D, FIG. 3E, FIG. 3F, FIG. 3G, and FIG. 3H show that HLA-G trimer promotes robust engineered surface expression. FIG. 3A shows schematic showing HLA-G isoform 1 components. FIG. 3B shows plasmid map for HLA-G dimer construct sequence introduced via lentivirus. FIG. 3C shows plasmid map for HLA-G trimer construct sequence introduced via lentivirus. FIG. 3D shows western blot results for B2M KO cells and B2M KO+GD cells probed for B2M. β-actin was used as a housekeeping protein. FIG. 3D shows immunofluorescent microscopy analysis of B2M expression in B2M KO+GD cells (fixed or fixed and permeabilized) and UDGT cells (fixed). Scale bars are 100 μm. FIG. 3E shows comparative live cell flow cytometry analysis of HLA expression for B2M KO+GD and UDGT cells. FIG. 3F shows flow cytometry analysis of HLA expression in WT (black), B2M KO (red), and UDGT cells (green). FIG. 3G shows comparative live cell flow cytometry analysis of HLA expression for UDGT cells treated with or without IFNγ. FIG. 3H shows that hPSCs were integrated with HLA-G trimer and then cultured in stem cell medium with or without IFNγ. Live cells were collected and processed for flow cytometry analyses of surface gene expression.

FIG. 4A, FIG. 4B, FIG. 4C, FIG. 4D, and FIG. 4E show generation of stable knockin universal donor hPSCs. FIG. 4A shows schematic knockin design and experimental execution. FIG. 4(B-C) shows flow cytometry analysis of B2M and HLA-E (FIG. 4B) or HLA-G (FIG. 44C) to determine KI efficiency seven days after transfection. FIGS. 4D-4E show flow cytometry analysis of HLA expression in WT (black), B2M KO (red), KI UDET (blue), and KI UDGT (green) for single cell derived clones for H9 (FIG. 4D) 6-9-9 (FIG. 4E) cells.

FIG. 5A, FIG. 5B, FIG. 5C, and FIG. 5D show in vitro immunogenicity of universal donor hPSCs. FIG. 5A shows schematic of cardiomyocyte differentiation. FIG. 5B shows analysis of day 6 cells for expression of cardiac progenitor marker ISL1. FIGS. 5C and 5D show CD8+T-cell (FIG. 5C) and NK cell (FIG. 5D) lysis of CPCs measured as percent specific lysis. Results for positive control CPCs treated with Triton-X and negative control untreated CPCs are incorporated into the calculation of percent specific lysis. Error bars represent standard error of the mean. *, **, ***, and **** indicate p values less than 0.05, 0.01, 0.001, and 0.0001 respectively. ns indicates values that are not significantly different.

FIG. 6 shows identification of successful B2M KO clones derived from 6-9-9 (top row) and H9 (bottom row). Wild type cells from each cell line and unstained control are also shown.

FIG. 7A, FIG. 7B, and FIG. 7C show that B2M KO cells remain pluripotent. FIG. 7A shows B2M KO hPSCs express pluripotency markers OCT4, NANOG, and SSEA4. FIG. 7B and 7C shows H9 (FIG. 7B) and 6-9-9 (FIG. 7C) B2M KO cells can differentiate to all three germ layers. Scale bars are 100 μm.

FIG. 8A, FIG. 8B, and FIG. 8C show the generation of the HLA-E trimer cells. FIG. 8A shows the plasmid map for HLA-E trimer construct sequence introduced via lentivirus. FIG. 8B shows flow cytometry analysis of HLA expression in WT (black), B2M KO (red), and HLA-E trimer (blue) cells. FIG. 8C shows flow cytometry analysis of HLA expression in H9 B2M KO cells transduced with HLA-E trimer treated with or without IFNg.

FIG. 9 shows flow cytometry analysis of engineered HLA expression in D8 cardiac progenitor cells derived from H9 UDET (top) and H9 UDGT (bottom).

FIG. 10A and FIG. 10B show flow cytometry analysis of HLA expression for 6-9-9 (FIG. 10A) and H9 (FIG. 10B) KI UDGT (top) and KI UDET (bottom) hPSCs treated with or without IFNg.

FIG. 11A and FIG. 11B show immunostaining for pluripotency genes for H9 KI cells (FIG. 11A) and 6-9-9 KI cells (FIG. 11B).

FIG. 12A, FIG. 12B, FIG. 12C, FIG. 12D, and FIG. 12E show the generation of B2M KO hPSCs. FIG. 12A shows the experimental schematic for the generation of B2M KO hPSCs. FIG. 12B shows brightfield and GFP images of B2M KO cells tranfected with p53DD (top) and without (bottom) 24 and 48 hours after nucleofection. Scale bars are 100 μm. FIG. 12C shows flow cytometry quantification of the GFP positive percentage of p53DD treated cells 48 hours after nucleofection. FIGS. 12 D and 12E show flow cytometry analysis of B2M expression for cells treated with p53DD one week post nucleofection (FIG. 12D) and for WT and selected single cell derived clones for H9 and 6-9-9 cells (FIG. 12E).

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### Definitions

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Reference will now be made in detail to the embodiments of the invention, examples of which are illustrated in the drawings and the examples. This invention may, however, be embodied in many different forms and should not be construed as limited to the embodiments set forth herein.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10”as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

The phrases “concurrent administration”, “administration in combination”, “simultaneous administration” or “administered simultaneously” as used herein, means that the compounds are administered at the same point in time or immediately following one another.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, at least about 75%, or at least about 90% complementary. See Kanehisa (1984) Nucl. Acids Res. 12:203.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells. A tissue-specific promoter may direct expression primarily in a desired tissue of interest, such as muscle, neuron, bone, skin, blood, specific organs, or particular cell types (e.g. stem cells). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8 (1), p. 466-472, 1988); SV40 enhancer; and the intron sequence between exons 2 and 3 of rabbit β-globin (Proc. Natl. Acad. Sci. USA., Vol. 78 (3), p. 1527-31, 1981). It will be appreciated by those skilled in the art that the design of the expression vector can depend on such factors as the choice of the host cell to be transformed, the level of expression desired, etc.

The term “recombinant” refers to a human manipulated nucleic acid (e.g., polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning-A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g., polynucleotides) are extremely unlikely to be found in nature. For instance, human manipulated restriction sites or plasmid vector sequences may flank or separate the promoter from the second nucleic acid (e.g., polynucleotide). One of skill will recognize that nucleic acids (e.g., polynucleotides) can be manipulated in many ways and are not limited to the examples above.

The term “expression cassette” or “vector” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning-A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P (N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

Nucleotide: The fundamental unit of nucleic acid molecules. A nucleotide includes a nitrogen-containing base attached to a pentose monosaccharide with one, two, or three phosphate groups attached by ester linkages to the saccharide moiety. The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T).

The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U).

As used throughout, by a “subject” (or a “host”) is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human. Administration of the therapeutic agents can be carried out at dosages and for periods of time effective for treatment of a subject.

The term “about” as used herein when referring to a measurable value such as an amount, a percentage, and the like, is meant to encompass variations of +20%, +10%, +5%, or +1% from the measurable value.

“Therapeutically effective amount” or “therapeutically effective dose” of a composition refers to an amount that is effective to achieve a desired therapeutic result. In some embodiments, a desired therapeutic result is reduction or clearance of a pathogen. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, cells, salts, esters, amides, proagents, active metabolites, isomers, fragments, analogs, and the like. When the term “therapeutic agent” is used, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially. Prophylactic treatments are administered to a subject prior to onset (e.g., before obvious signs of a cardiac disorder), during early onset (e.g., upon initial signs and symptoms of a cardiac disorder), or after an established development of a cardiac disorder. Prophylactic administration can occur for several days to years prior to the manifestation of symptoms of a disorder

The term “polypeptide” refers to a compound made up of a single chain of D-or L-amino acids or a mixture of D-and L-amino acids joined by peptide bonds.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “polynucleotide” refers to a single or double stranded polymer composed of nucleotide monomers.

### Compositions and Methods

Disclosed herein are universal stem cells and methods of making the cells. In some examples, genome editing tools are used to specifically knock out beta-2-Microglobulin (B2M) gene to prevent T cell-mediated lysis towards the transplanted cells. In some examples, a recombinant polynucleotide that comprises a polynucleotide sequence encoding a HLA-E or a HLA-G polypeptide is introduced into the universal stem cells to prevent NK cell-mediated lysis towards the stem cells once transplanted into recipients. Introduction of the recombinant polynucleotides into universal stem cells confers stable and high expression of the HLA proteins (for example, HLA-G and/or HLA-E) encoded by the recombinant polynucleotides on cell surface.

Accordingly, in some aspects, disclosed herein is a recombinant polynucleotide comprising a first polynucleotide sequence encoding a first beta-2-Microglobulin (B2M) polypeptide or a fragment thereof; a second polynucleotide sequence encoding a peptide (e.g., a nonamer) or a fragment thereof; a third polynucleotide sequence encoding a second B2M polypeptide or fragment thereof; and a fourth polynucleotide sequence encoding a human leukocyte antigen (HLA)-E polypeptide or a HLA-G polypeptide or a fragment thereof.

In some embodiments, the first B2M polypeptide is a B2M signaling peptide or a fragment thereof. In some embodiments, the second B2M polypeptide is a B2M polypeptide that lacks the domain of B2M signaling peptide.

Accordingly, in some aspects, disclosed herein is a recombinant polynucleotide comprising a first polynucleotide sequence encoding a beta-2-Microglobulin (B2M) signaling peptide or a fragment thereof; a second polynucleotide sequence encoding a peptide (e.g., a nonamer) or a fragment thereof; a third polynucleotide sequence encoding a B2M polypeptide that lacks signaling peptide; and a fourth polynucleotide sequence encoding a human leukocyte antigen (HLA)-E or HLA-G polypeptide or a fragment thereof.

It should be understood and herein contemplated that the first polynucleotide sequence, the second polynucleotide sequence, the third polynucleotide sequence, and the fourth polynucleotide sequence can be linked by linkers in any order. In some embodiments, the linker comprises a nucleic acid sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 7 or SEQ ID NO: 9. In some embodiments, the linker sequence encodes a polypeptide sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 8, 10 or 27.

The term “signaling peptide” or “signal peptide” herein refers to a short peptide (for example, about 10 to 30 amino acid residues in length) normally present at the N-terminus or the C-terminus of a newly synthesized protein, directing the protein to secretory pathway.

In some embodiments, the B2M signaling peptide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 2 or SEQ ID NO: 17 or a fragment thereof. In some embodiments, the first polynucleotide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 1 or SEQ ID NO: 16 or a fragment thereof.

In some embodiments, the B2M polypeptide lacking signaling peptide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 4 or SEQ ID NO: 19 or a fragment thereof. In some embodiments, the third polynucleotide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 3 or SEQ ID NO: 18 or a fragment thereof.

In some examples, the fourth polynucleotide sequence encodes a HLA-G polypeptide or a fragment thereof. HLA-G belongs to the HLA class I heavy chain paralogues. This class I molecule is a heterodimer consisting of a heavy chain and a light chain (beta-2 microglobulin). HLA-G can be expressed on fetal derived placental cells. In some embodiments, the HLA-G polypeptide is the heavy chain of HLA-G. In some embodiments, the HLA-G polypeptide lacks signaling peptide. In some embodiments, the HLA-G polypeptide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 21 or a fragment thereof. In some embodiments, the fourth polynucleotide sequence comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 20 or a fragment thereof.

HLA-G requires the binding of a peptide for molecule stabilization and cell surface expression. The disclosed recombinant polynucleotide encodes a trimer protein comprising a HLA-G polypeptide, B2M, and a peptide (for example, a nonamer), conferring stable cell surface expression of the HLG-A polypeptide. In some embodiments, the peptide is a nonamer. In some embodiments, the peptide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 15 or a fragment thereof. In some embodiments, the second polynucleotide sequence encoding the peptide (e.g., the nonamer) comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 14 or a fragment thereof.

In some embodiment, the recombinant polynucleotide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 22 or a fragment thereof.

In some examples, the fourth polynucleotide sequence encodes a HLA-E polypeptide or a fragment thereof. HLA-E belongs to the HLA class I heavy chain paralogues. This class I molecule is a heterodimer consisting of a heavy chain and a light chain (beta-2 microglobulin). In some embodiments, the HLA-E polypeptide is the heavy chain of HLA-E. In some embodiments, the HLA-E polypeptide lacks signaling peptide. In some embodiments, the HLA-E polypeptide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 12 or a fragment thereof. In some embodiments, the fourth polynucleotide sequence comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 11 or a fragment thereof.

HLA-E most often binds peptides processed from the signal peptide sequences of other class I HLAs. HLA-E expression is reduced in the absence of other HLA molecule expression. Nonamer peptides derived from the signal peptide sequence of a class I HLA (e.g., HLA-C or HLA-G) induce strong CD94/NKG2A inhibitory immune interactions. The herein disclosed recombinant polynucleotide encodes a trimer protein comprising a HLA-G polypeptide, B2M, and a peptide (for example, a nonamer derived from the signal peptide sequence of a class I HLA), conferring stable cell surface expression of the HLG-A polypeptide. In some embodiments, the peptide is a noanmer is derived from HLA-C or HLA-G. In some embodiments, the peptide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 6 or a fragment thereof. In some embodiments, the second polynucleotide sequence encoding the peptide (e.g., the nonamer) comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 5 or a fragment thereof.

In some embodiment, the recombinant polynucleotide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 13 or a fragment thereof.

Also disclosed herein is a recombinant polynucleotide comprising a first polynucleotide sequence encoding a beta-2-Microglobulin (B2M) signaling peptide or a fragment thereof; and a second polynucleotide sequence encoding a human leukocyte antigen (HLA)-E or HLA-G polypeptide or a fragment thereof.

It should be understood and herein contemplated that the first polynucleotide sequence and the second polynucleotide sequence can be linked by a linker in any order. In some embodiments, the linker comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 7 or SEQ ID NO: 9. In some embodiments, the linker sequence encodes a polypeptide sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 8, 10 or 27.

In some embodiments, the B2M polypeptide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 26 or SEQ ID NO: 30 or a fragment thereof. In some embodiments, the second polynucleotide sequence comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 25 or SEQ ID NO: 29 or a fragment thereof.

In some embodiments, the second polynucleotide sequence encodes a HLA-E polypeptide or a fragment thereof. In some embodiments, the second polynucleotide encodes a HLA-G polypeptide or a fragment thereof.

In some embodiments, the HLA-E polypeptide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 24 or a fragment thereof. In some embodiments, the second polynucleotide sequence comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 23 or a fragment thereof.

In some embodiments, the HLA-G polypeptide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 32 or a fragment thereof. In some embodiments, the second polynucleotide sequence comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 31 or a fragment thereof.

In some embodiment, the recombinant polynucleotide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 28 or SEQ ID NO: 33 or a fragment thereof.

The recombinant polynucleotide disclosed herein can be contained in a vector that can be used to deliver the recombinant polynucleotide to cells, either in vitro or in vivo. Accordingly, in some aspects, disclosed herein is a vector comprising the recombinant polynucleotide disclosed herein. The vector can be an Adeno-Associated Virus Integration Site 1 (AAVS1) locus targeting vector. In some embodiments, the vector comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 34 or 35 or a fragment thereof. In some examples, the vector comprises the recombinant polynucleotide that comprises a HLA-E coding polynucleotide sequence, and wherein the vector comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 34 or a fragment thereof. In some examples, the vector comprises the recombinant polynucleotide that comprises a HLA-G coding polynucleotide sequence, and wherein the vector comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 35 or a fragment thereof.

The vectors and the delivery methods can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Transfer vectors can be any nucleotide construction used to deliver genes into cells, or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed polynucleotides into the cell without degradation and include a promoter yielding expression of the gene in the cells into which it is delivered. In some embodiments, the polypeptides are derived from either a virus or a retrovirus. Viral vectors can be, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. A preferred embodiment is a viral vector which has been engineered so as to suppress the immune response of the host organism, elicited by the viral antigens.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsulation, and promoters to control the transcription and replication of the viral genome. When engineered as vectors, viruses typically have one or more of the early genes removed and a gene or gene/promotor cassette is inserted into the viral genome in place of the removed viral DNA.

In some embodiments, the polynucleotide disclosed herein is contained in an adeno-associated virus (AAV) vector or a lentiviral vector (including, but not limited to simian immunodeficiency virus (SIV), human immunodeficiency virus 1 (HIV-1), human immunodeficiency virus 2 (HIV-2), and feline immunodeficiency virus). This defective parvovirus is a preferred vector because it can infect many cell types and is nonpathogenic to humans.

### Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468,(1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

### A) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids into the cell without degradation and include a promoter yielding expression of the gene in the cells into which it is delivered. In some embodiments the polynucleotides are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

### (1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

### (2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

### (3) Adeno-associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### (4) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8:33-41, 1994; Cotter and Robertson, Curr Opin Mol Ther 5:633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable. The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

### B) Non-nucleic Acid-based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed polynucleotides, constructs, or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No.4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, MD), SUPERFECT (Qiagen, Inc. Hilden, Germany) and TRANSFECTAM (Promega Biotec, Inc., Madison, WI), as well as other liposomes developed according to procedures standard in the art. In addition, the disclosed nucleic acid or vector can be delivered in vivo by electroporation, the technology for which is available from Genetronics, Inc. (San Diego, CA) as well as by means of a SONOPORATION machine (ImaRx Pharmaceutical Corp., Tucson, AZ).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

### C) In Vivo/Ex Vivo

As described above, the compositions can be administered in a pharmaceutically acceptable carrier and can be delivered to the subjects' cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

### Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### A) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273:113 (1978)). The immediate early promoter of the human cytomegalovirus is conveniently obtained as a HindIII E restriction fragment (Greenway, P. J. et al., Gene 18:355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78:993 (1981)) or 3′ (Lusky, M. L., et al., Mol. Cell Bio. 3:1108 (1983)) to the transcription unit.

Furthermore, enhancers can be within an intron (Banerji, J. L. et al., Cell 33:729 (1983)) as well as within the coding sequence itself (Osborne, T. F., et al., Mol. Cell Bio. 4:1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin,-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

### Universal Stem Cell

In some aspects, disclosed herein is a universal stem cell for differentiation that are pluripotent. The universal comprises the recombinant polynucleotide disclosed herein for the expression of HLA-G or HLG-E on the cell surface. The universal stem cells comprise the recombinant polynucleotide disclosed herein. In some embodiments, the universal stem cell is an induced pluripotent stem cell.

The term ‘stem cells’ as used herein refers to undifferentiated cells defined by their ability at the single cell level to both self-renew and differentiate to produce progeny cells, including self-renewing progenitors, non-renewing progenitors, and terminally differentiated cells. Stem cells have the ability to divide for indefinite periods in culture. Stem cells are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts. Stem cells are categorized as somatic (adult) stem cells or embryonic stem cells. A somatic stem cell is an undifferentiated cell found in a differentiated tissue that can renew itself (clonal) and (with certain limitations) differentiate to yield all the specialized cell types of the tissue from which it originated.

The term ‘induced pluripotent stem cells’, commonly abbreviated as iPSC, as used herein refers to somatic (adult) cells reprogrammed to enter an embryonic stem cell-like state by being forced to express factors important for maintaining the “stemness” of embryonic stem cells. Typically, iPSC are artificially prepared from a non-pluripotent cell, (i.e. adult somatic cell, or terminally differentiated cell) such as fibroblast, a hematopoietic cell, a myocyte, a neuron, an epidermal cell, or the like, by introducing into or otherwise contacting the cell with reprogramming factors.

The term “pluripotency” as used herein is generally understood by the skilled person and refers to an attribute of a pluripotent stem cell that has the potential to differentiate into all cells constituting one or more tissues or organs, for example, any of the three germ layers: endoderm (e.g. interior stomach lining, gastrointestinal tract, the lungs), mesoderm (e.g. heart, muscle, bone, blood, urogenital tract), or ectoderm (e.g. epidermal tissues and nervous system). 129. Accordingly, in some aspects, disclosed herein is a universal stem cell comprising a recombinant polynucleotide comprising a first polynucleotide sequence encoding a beta-2-Microglobulin (B2M) signaling peptide or a fragment thereof; a second polynucleotide sequence encoding a nonamer or a fragment thereof; a third polynucleotide sequence encoding a B2M polypeptide that lacks signaling peptide; and a fourth polynucleotide sequence encoding a human leukocyte antigen (HLA)-E or HLA-G polypeptide or a fragment thereof.

In some embodiments, the universal stem cell comprises a deletion in a B2M gene or a fragment thereof (e.g., a deletion of exon 1, exon 2, and/or exon 3 or a fragment thereof). In some embodiments, the universal stem cell comprises a polynucleotide encoding a Cas9 and a polynucleotide encoding a guide RNA. In some embodiments, the universal stem cell comprises a polynucleotide encoding a Cas9 and a guide RNA. In some embodiments, the guide RNA targets the beta-2-Microglobulin gene or a fragment thereof. In some embodiments, the guide RNA comprises a sequence 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 37 or 38 or a fragment thereof. In some embodiments, the polynucleotide encoding the guide RNA comprises a sequence 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 37 or 38 or a fragment thereof.

In some embodiments, the B2M signaling peptide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 2 or SEQ ID NO: 17 or a fragment thereof. In some embodiments, the first polynucleotide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 1 or SEQ ID NO: 16 or a fragment thereof.

In some embodiments, the B2M polypeptide that lack signaling peptide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 4 or SEQ ID NO: 19 or a fragment thereof. In some embodiments, the third polynucleotide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 3 or SEQ ID NO: 18 or a fragment thereof.

In some embodiments, the HLA-G polypeptide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 21 or a fragment thereof. In some embodiments, the fourth polynucleotide sequence comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 20 or a fragment thereof.

In some embodiments, the nonamer comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 15 or a fragment thereof. In some embodiments, the second polynucleotide sequence encoding the peptide (e.g., the nonamer) comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 14 or a fragment thereof. 135. In some embodiment, the recombinant polynucleotide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 22 or a fragment thereof.

In some examples, the fourth polynucleotide sequence encodes a HLA-E polypeptide or a fragment thereof. In some embodiments, the HLA-E polypeptide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 12 or a fragment thereof. In some embodiments, the fourth polynucleotide sequence comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 11 or a fragment thereof.

In some embodiments, the nonamer comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 6 or a fragment thereof. In some embodiments, the second polynucleotide sequence encoding the peptide (e.g., the nonamer) comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 5 or a fragment thereof.

In some embodiment, the recombinant polynucleotide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 13 or a fragment thereof.

In some aspects, disclosed herein is a universal stem cell comprising a recombinant polynucleotide comprising a first polynucleotide sequence encoding a beta-2-Microglobulin (B2M) signaling peptide or a fragment thereof; and a second polynucleotide sequence encoding a human leukocyte antigen (HLA)-E or HLA-G polypeptide or a fragment thereof.

In some embodiments, the B2M polypeptide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 26 or SEQ ID NO: 30 or a fragment thereof. In some embodiments, the second polynucleotide sequence comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 25 or SEQ ID NO: 29 or a fragment thereof.

In some embodiments, the second polynucleotide sequence encodes a HLA-E polypeptide or a fragment thereof. In some embodiments, the second polynucleotide encodes a HLA-G polypeptide or a fragment thereof.

In some embodiments, the HLA-E polypeptide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 24 or a fragment thereof. In some embodiments, the second polynucleotide sequence comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 23 or a fragment thereof.

In some embodiments, the HLA-G polypeptide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 32 or a fragment thereof. In some embodiments, the second polynucleotide sequence comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 31 or a fragment thereof.

In some embodiment, the recombinant polynucleotide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 28 or SEQ ID NO: 33 or a fragment thereof.

The polynucleotides disclosed herein can be contained in the vector disclosed herein that can be used to deliver the polynucleotide to stem cell. It should be understood that the expression of HLA-E and HLA-G decreases during the stem cell differentiation. Therefore, in some embodiments, the recombinant polynucleotides disclosed herein are knocked in at the AAVS1 locus of the universal stem cell. Accordingly, the vector can be an AAVSI locus targeting vector. In some embodiments, the vector comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to SEQ ID NO: 34 or 35 or a fragment thereof.

The safe harbor knockin at the AAVSI locus is CRISPR-Cas9 mediated. Accordingly, the universal stem cell can further comprise a polynucleotide encoding a Cas9 and a polynucleotide encoding a guide RNA.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. CRISPR systems are known in the art. See, e.g., U.S. Pat. No. 8,697,359, incorporated by reference herein in its entirety.

A gRNA is a component of the CRISPR/Cas system. A “gRNA” (guide ribonucleic acid) herein refers to a fusion of a CRISPR-targeting RNA (crRNA) and a trans-activation crRNA (tracrRNA), providing both targeting specificity and scaffolding/binding ability for Cas9 nuclease. A “crRNA” is a bacterial RNA that confers target specificity and requires tracrRNA to bind to Cas9. A “tracrRNA” is a bacterial RNA that links the crRNA to the Cas9 nuclease and typically can bind any crRNA. The sequence specificity of a Cas DNA-binding protein is determined by gRNAs, which have nucleotide base-pairing complementarity to target DNA sequences. The native gRNA comprises a Specificity Determining Sequence (SDS), which specifies the DNA sequence to be targeted. At least a portion of the target DNA sequence is complementary to the SDS of the gRNA. For Cas9 to successfully bind to the DNA target sequence, a region of the target sequence is complementary to the SDS of the gRNA sequence and is immediately followed by the correct protospacer adjacent motif (PAM) sequence (e.g., NGG or NG for Cas9 used herein). In some embodiments, an SDS is 100% complementary to its target sequence. In some embodiments, the SDS sequence is less than 100% complementary to its target sequence and is, thus, considered to be partially complementary to its target sequence. For example, a targeting sequence may be 99%, 98%, 97%, 96%, 95%, 94%, 93%, 92%, 91%, or 90% complementary to its target sequence.

In some embodiments, the universal stem cell further comprises a p53 dominant-negative (p53 DD) polypeptide. In some embodiments, the polynucleotide encoding a p53 DD polypeptide comprises a sequence at least about 80% identity to SEQ ID NO: 36.

The universal stem cell can have a deletion in B2M host gene or a fragment thereof to prevent T cell-mediated lysis. The universal stem cell can have a deletion in any of the four exons (exon 1, exon 1, exon 3, and/or exon 4) of B2M host gene.

Also disclosed herein is a method of making a universal stem cell, comprising transducing the recombinant polynucleotide disclosed herein into the universal stem cell, and culturing the transduced universal stem cell, wherein the recombinant polynucleotide comprises a first polynucleotide sequence encoding a beta-2-Microglobulin (B2M) signaling peptide or a fragment thereof; a second polynucleotide sequence encoding a nonamer or a fragment thereof; a third polynucleotide sequence encoding a B2M polypeptide that lacks signaling peptide; and a fourth polynucleotide sequence encoding a HLA-E or HLA-G polypeptide or a fragment thereof.

Also disclosed herein is a method of a making a cardiomyocyte, comprising making a universal stem cell by the method disclosed herein for making a universal stem cell; culturing the universal stem cell (e.g., for about 5 days, 6 days, 7 days, 8 days, 9 days, 10 days, 11 days, 12days, 13 days, 14 days, 15 days, 18 days, 20 days, 25 days, 30 days, 60 days, or 100 days) in a cell culture media to differentiate the universal stem cell into a cardiomyocyte; and harvesting the differentiated cardiomyocyte. In some embodiments, the universal stem cell is cultured for at least about 12 days to differentiate the universal stem cell into a cardiomyocyte.

In the present invention, the term ‘differentiation’ as used herein refers to a biological process whereby an unspecialized PESC or iPSC acquires the features of a specialized cell (e.g., a somatic cell) such as a heart cell (e.g. cardiomyocyte), liver cell, or muscle cell under controlled conditions in in vitro culture. Differentiation is controlled by the interaction of a cell's genes with the physical and chemical conditions outside the cell, usually through signaling pathways involving proteins embedded in the cell surface. In certain embodiments, pluripotent stem cells (e.g. PESC or iPSC) can be exposed to the culture medium compositions and methods of the invention so as to promote differentiation of pluripotent stem cells into foetal-like cardiomyocytes. Cardiac differentiation can be detected by the use of markers selected from, but not limited to, NKX2-5, GATA4, myosin heavy chain, myosin light chain, alpha-actinin, troponin, and tropomyosin (Burridge et al (2012) Stem Cell, Vol. 10 (1): 16-28, U.S. Patent Application Publication No. US2013/0029368, which is incorporated herein by reference in its entirety).

Accordingly, also disclosed herein is a method of making a cell (e.g., a neuronal cell, a pancreatic cell, a cardiac cell, an endothelial cell, or an immune cell), comprising making a universal stem cell by the method disclosed herein disclose herein and differentiating the universal stem cell into the cell.

Also disclosed herein are uses of the differentiated cells (e.g., a neuronal cell, a pancreatic cell, a cardiac cell, an endothelial cell, or an immune cell) for treating a neurological disorder, diabetes, a cardiac disorder, a vascular disorder, or an immunological disorder in a subject comprising transplanting the differentiated cells into the subject.

The skilled person is aware of various methods to obtain stem cell, (e.g., iPSC or embryonic stem cell), derived cardiomyocytes. When stem cells are removed from differentiation suppression conditions and/or when grown in suspension aggregates, called embryoid bodies, spontaneous differentiation to cells of the three germ layers occurs. Cardiomyocytes originate from the mesodermal germ layer and differentiation of stem cells into cardiomyocytes thus requires efficient differentiation toward the mesodermal lineage. Such directed differentiation toward the cardiac lineage is mainly achieved by several strategies, including the formation of embryoid bodies in the presence of growth factors and repressors known to influence heart development (see, for example Kehat et al. Clin. Invest. 2001; 108, 407-414), reliance on the influence of endoderm on cardiac differentiation during embryogenesis (Mummery et al., Circulation. 2003; 107, 2733-2740). Methods for stem cell differentiation are known in the art. See, e.g., U.S. Pat. Nos. 10,696,947 and 9,395,354, incorporated by reference herein in their entireties.

In some aspects, disclosed herein is a method for treating a cardiac disorder in a subject in need, comprising making a universal stem cell by the method disclosed herein for making a universal stem cell; culturing the universal stem cell (e.g., for about 5 days, 6 days, 7 days, 8 days, 9 days, 10 days, 11 days, 12 days, 13 days, 14 days, 15 days, 18 days, 20 days, 25 days, 30 days, 60 days, or 100 days) in a cell culture media to differentiate the universal stem cell into a cardiomyocyte; and transplanting the cardiomyocyte into the subject. In some embodiments, the universal stem cell is cultured for at least about 12 days to differentiate the universal stem cell into a cardiomyocyte.

In some embodiments, the universal stem cell is derived from the subject. In some embodiments, the universal stem cell is not derived from the subject.

In one aspect, the disclosed methods can be employed 10, 9, 8, 7, 6, 5, 4, 3, 2 years, 12, 11, 10, 9, 8, 7, 6, 5, 4, 3, 2 months, 30, 29, 28, 27, 26, 25, 24, 23, 22, 21, 20, 19, 18, 17, 16, 15, 14, 13, 12, 11, 10, 9, 8, 7, 6, 5, 4, 3 days, 60, 48, 36, 30, 24, 18, 15, 12, 10, 9, 8, 7, 6, 5, 4, 3, 2 hours, 60, 45, 30, 15, 10, 9, 8, 7, 6, 5, 4, 3, 2, or 1 minute prior to onset of a cardiac disorder; or 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 days, 3, 4, 5, 6, 7, 8, 9, 10, 11, 12 months, 2, 3, 4, 5, 6, 7, 8, 9, 10 or more years after onset of a cardiac disorder.

### EXAMPLES

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. While the invention has been described with reference to particular embodiments and implementations, it will be understood that various changes and additional variations may be made and equivalents may be substituted for elements thereof without departing from the scope of the invention or the inventive concept thereof. In addition, many modifications may be made to adapt a particular situation or device to the teachings of the invention without departing from the essential scope thereof. Such equivalents are intended to be encompassed by the following claims. It is intended that the invention not be limited to the particular implementations disclosed herein, but that the invention will include all implementations falling within the scope of the appended claims.

### Example 1: Introduction

Heart transplantation is one of the only long-term clinical options for end stage heart failure, which occurs when existing cardiomyocytes (CMs) become damaged and unable to function or die. However, there are many issues with allogeneic transplants. Graft rejection is the primary concern following organ transplantation, necessitating the administration of immunosuppressive therapy for the duration of a patient's life. Immunosuppressive therapy not only leaves a recipient susceptible to common infections, but can also lead to the development of diabetes, renal failure, or cardiovascular disease. The economic burden of this therapy is immense, with immunosuppressive therapy for new transplant recipients in the US in 2017 alone costing an estimated one trillion dollars. Additionally, the demand for suitable donor matched organs far outweighs the supply. With a rapidly aging population, this is not a sustainable model of care, and as such, we sought to address this problem by engineering a universal donor cell line as an alternative to organ transplant therapies.

Human pluripotent stem cells (hPSCs), both embryonic and induced (iPSCs), offer unlimited self-renewal and capacity to differentiate into any desired therapeutic cell type, making these cells an ideal candidate for a universal donor cell line. iPSC technology provides the ability to develop an autologous source of donor cells, and chemically defined and efficient methods were developed for generating CMs from hPSCs. Unfortunately, establishing patient-specific iPSCs is time consuming, costly, and precludes off-the-self applicability. Moreover, murine studies have shown that while this strategy can be effective for some tissue types such as skin, it is ineffective for CMs, resulting in immune rejection. In light of the special immunogenic properties of CMs, alternative strategies needed to be considered.

A myriad of efforts have been made to determine how to evade immune rejection, most of which center on HLA molecule expression. Due to the high degree of polymorphic variations in the peptide binding cleft of HLAs A, B, and C, CD8+ cytotoxic T cells can distinguish between “self” from “non-self” cells, resulting in lysis of foreign cells and ultimately to graft rejection. In order to circumvent CD8+ T cell mediated graft rejection, some groups have specifically removed these three classical HLAs (A, B, and C). A more streamlined approach, however, is to biallelically knockout (KO) the conserved B2M gene, that can cause complete KO of all class I HLAs, and therefore can create a cell line that would not elicit CD8+ T cell rejection.

Immunoengineering strategies that completely remove class I HLAs, however, become susceptible to natural killer (NK) cell lysis, through what is known as the missing self response. To prevent this, some groups have explored the expression of CD47, a surface marker expressed at the maternal-fetal interface, which is an example of a naturally occurring hemi-allogeneic “graft”. This strategy resulted in reduced NK cell activation in an HLA class I and II null background. Expression of immunoregulatory markers PD-L1 and CTLA4-Ig, popular cancer immunotherapy targets, have also been tested to mitigate T-cell attack, but both of these markers are insufficient on their own and require the expression of additional immunoregulatory proteins to obtain immune protection. Genetic KO of certain HLA genes A and B combined with a KO of one allele of HLA-C would result in the evasion of CD8+ T-cell and NK cell mediated lysis. This generation of an HLA-C haplozygous donor cell line is a more stringent approach to donor matching by HLA homozygous donor cells, which has been attempted in primate models with iPSC derived CM transplantation and retained the requirement for immunosuppressive therapy to prevent rejection.

The study herein shows the generation of the universal donor hPSCs and the in vitro testing of their immunogenicity as differentiated cardiomyocytes. A data driven and cardiac specific approach was developed to generating a universal donor stem cell line using a two part CRISPR-Cas9 mediated genetic immunoenginnering method. All class I HLA expression was removed and a direct comparison of the in vitro immunogenicity of HLA-E and HLA-G overexpression was performed. The design of the HLA proteins was optimized to obtain stable and high surface level expression.

### Example 2. Design and Selection of Hypoimmunogenic Molecules for Universal Donor Cells

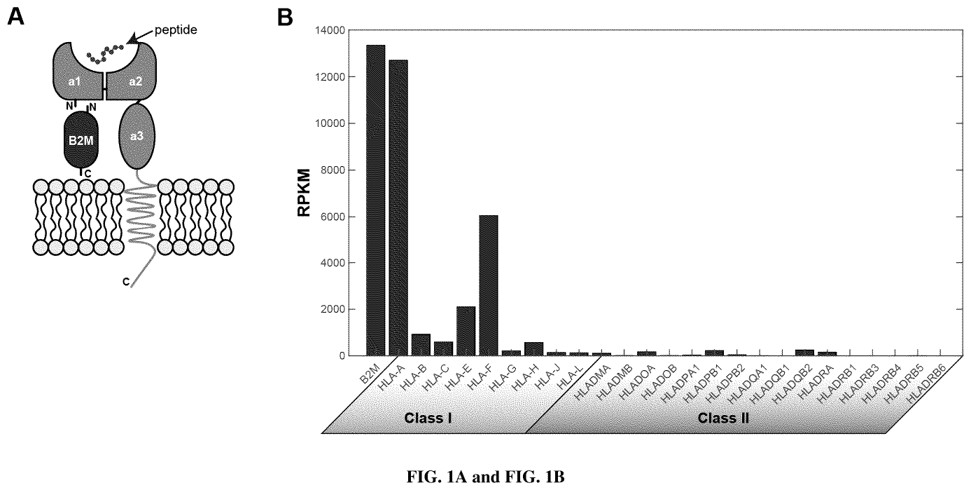

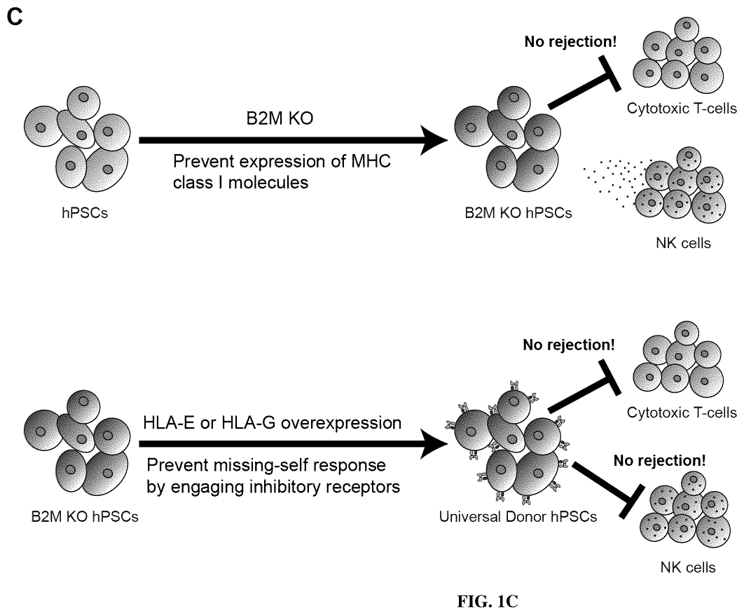

To inform the design of the universal donor cell for cardiac regenerative therapy, the HLA expression profile of mature in vitro hPSC derived CMs was first examined. RNA sequencing data revealed high expression of class I HLA molecules, specifically HLA-A,-B, and-C, with very high HLA-A expression, with no class II HLA expression (FIG. 1B). Class I HLA molecules are trimeric, consisting of a specific HLA heavy chain that noncovalently binds to a conserved beta-2-microglobulin (B2M) and an antigen peptide (FIG. 1A). As such, the universal donor stem cell design involved immunoengineered HLA expression in two parts (FIG. 1C). First, all class I HLA expression was removed by Cas9-mediated biallelic KO of the B2M gene to prevent CD8+ T-cell lysis. Then, examined candidate proteins for inducing hypoimmunogenicity, and found HLA-E and HLA-G to both be the most ideal. They are both minimally polymorphic and not known to bind to or be recognized by CD8 receptors. The expression of HLA-E or HLA-G cannot trigger CD8+ T-cell activation and can prevent the NK missing-self response.

HLA-G expression is tissue restricted and has been implicated in the suppression of T-cell, B-cell, and NK cell activation. HLA-G is notably expressed at the maternal-fetal interface during pregnancy, which has led to the theory that it plays a role in immune tolerance. It is also upregulated in tumor cells as a mechanism of immune evasion. Furthermore, HLA-G was shown to be upregulated in heart transplants, indicating its expression was associated with decreased incidence of acute and chronic rejection, an improvement for transplant outcomes. Based on this data, HLA-G was selected as a candidate for mediating immune tolerance for cardiac regeneration with universal donor stem cells.

HLA-E has well-established inhibitory and activation effects on NK cells resulting from interactions with CD94/NKG2 receptors on NK cells. Cells expressing HLA-G showed increased HLA-E expression, most notably at the maternal-fetal interface indicating a role in immune privilege. Upregulated expression of HLA-E has also been implicated with age-related immune evasion of senescent dermal fibroblasts. Recently, the HLA-E/NKG2A interaction was designated as an immune checkpoint and investigated as a target for cancer immunotherapies due to the increased expression of HLA-E on tumor surfaces. Expression of HLA-E in porcine cells has resulted in the ability of those cells to evade human NK cell mediated lysis, signifying the inhibitory trigger is able to transcend species mismatch. Therefore, HLA-E was selected as a second candidate for further examination in the generation of universal donor stem cells, specifically in a cardiac regenerative context.

### Example 3. Generation and Characterization of B2M KO hPSCs

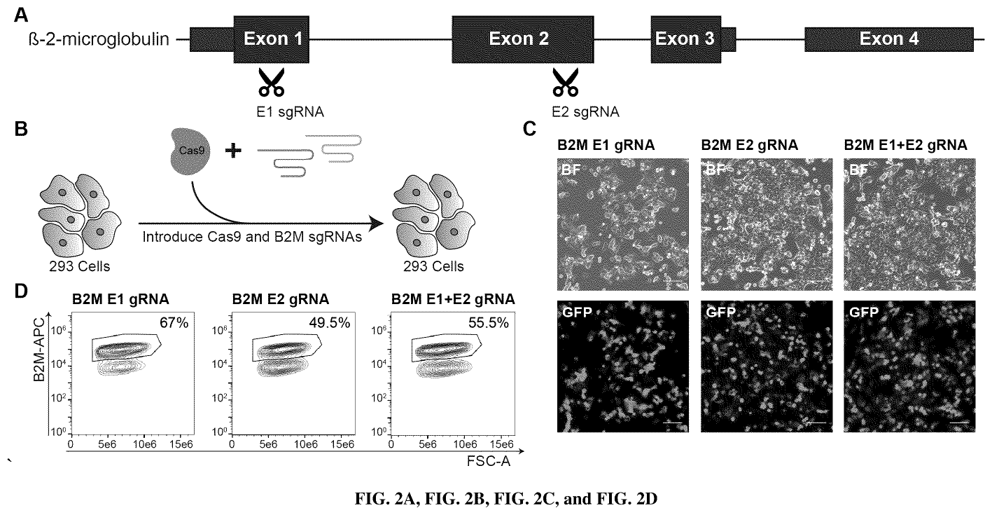

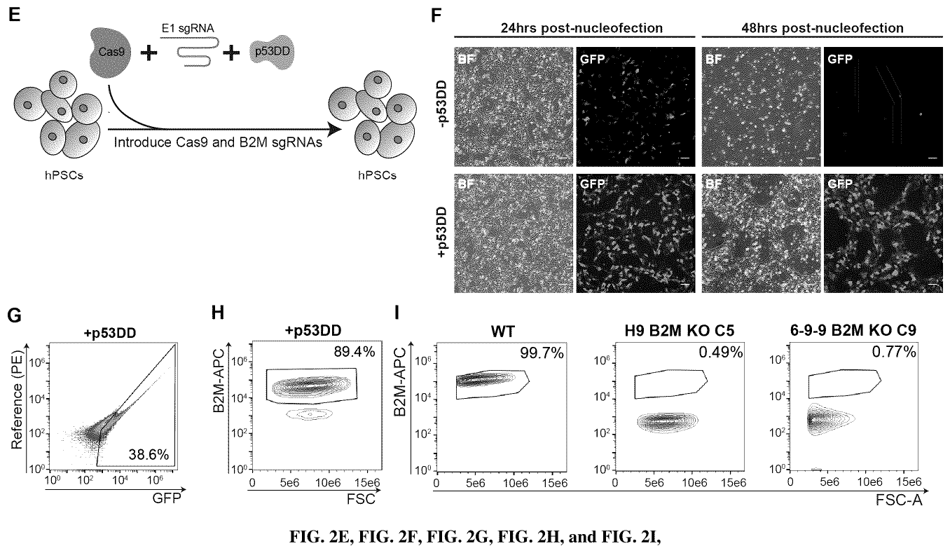

B2M KO hPSCs were generated using CRISPR-Cas9 genome editing techniques. B2M has four exons with the start codon in exon 1 and the stop codon in exon 3. We designed two different single guide RNAs (sgRNAs), to target either exon 1 (E1 gRNA) or exon 2 (E2 gRNA), for introducing a double stranded break (FIG. 2A). Before trying to generate a KO in hPSCs, which can be difficult to transfect, the efficacy of the sgRNAs were first tested in HEK 293 cells. HEK 293 cells were lipofected with a plasmid encoding Cas9 and GFP and included plasmids encoding the E1 gRNA, the E2 gRNA, or both (FIG. 2B). The day after transfection, most of the cells in all three conditions were GFP positive indicating successful transfection (FIG. 2C). The cells were maintained for one week after transfection to allow any existing B2M protein to be degraded. Then the percentage of the population with a successful B2M KO was measured via flow cytometry analysis of B2M expression (FIG. 2D). All three conditions resulted in B2M KO cells and even the single sgRNAs were successful (FIG. 2D). Accordingly, the E1 gRNA was selected for use in hPSCs due to its target location near the start codon, which prevents truncated protein expression.

To generate the B2M KO hPSCs two hPSC cell lines (H9 and 6-9-9) were transfected with plasmids encoding Cas9 and GFP, the selected sgRNA (E1 gRNA), and p53DD (FIG. 2E). p53DD was included because transient expression has been shown to improve stem cell survival by inhibiting p53 mediated apoptosis and cell cycle arrest following the formation of a double stranded break during Cas9 editing. In this system, the inclusion of p53DD greatly improved cell survival from 24 to 48 hours following transfection using nucleofection technology (FIG. 2F). With p53DD added, a transfection efficiency of 38% and KO efficiency of 10% was measured one week after transfection (FIGS. 2G and 2H). Single cell derived colony selection was performed and these clones were analyzed by flow cytometry to detect clones that lost B2M expression. 3 KO clones were identified with the 6-9-9 cell line and 4 were identified with the H9 cells, and one clone of each was selected for further analysis and use (FIG. 21, FIG. 6).

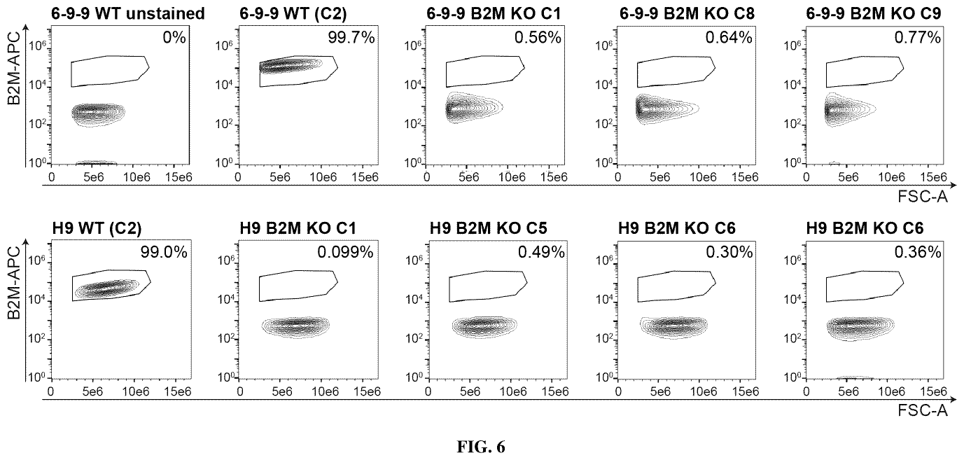

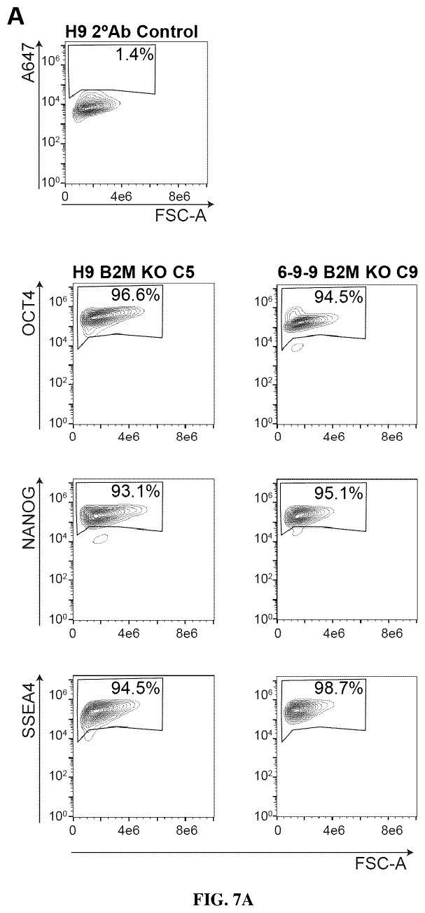

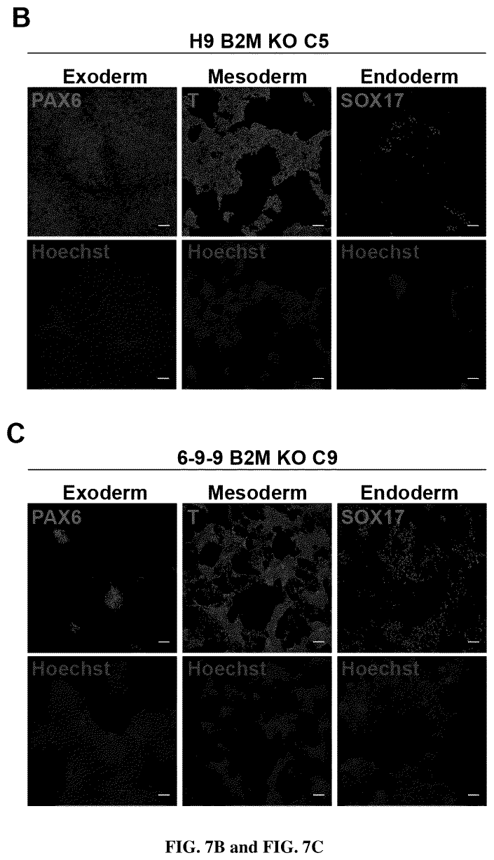

To further characterize the selected B2M KO hPSC clones, flow cytometry analysis was performed for pluripotency markers and over 90% of the cells expressed OCT4, NANOG, and SSEA4 were found (FIG. 7A). The cells were differentiated to each of the three germ layers and differentiation potential was confirmed by immunofluorescent microscopy analysis of PAX6, T and SOX17 for the ectoderm, mesoderm, and endoderm respectively (FIGS. 7B and 7C). Both cell lines were able to differentiate successfully to all three germ layers further denoting maintained pluripotency. TOPO-TA cloning was performed to determine the sequence of both alleles of B2M in these selected B2M KO clones. The non-homologous end joining repair mechanism resulted in the generation of a 1-20 base pair deletion yielding frame shift mutations and premature stop codons (FIG. 2J). The 6-9-9 cells showed different indels for each allele whereas the H9 B2M KO cells had the same 10bp deletion in both alleles (FIG. 2J). Together, these data illustrate the successful generation of B2M KO hPSCs.

### Example 4. Design and Characterization of Universal Donor hPSCs

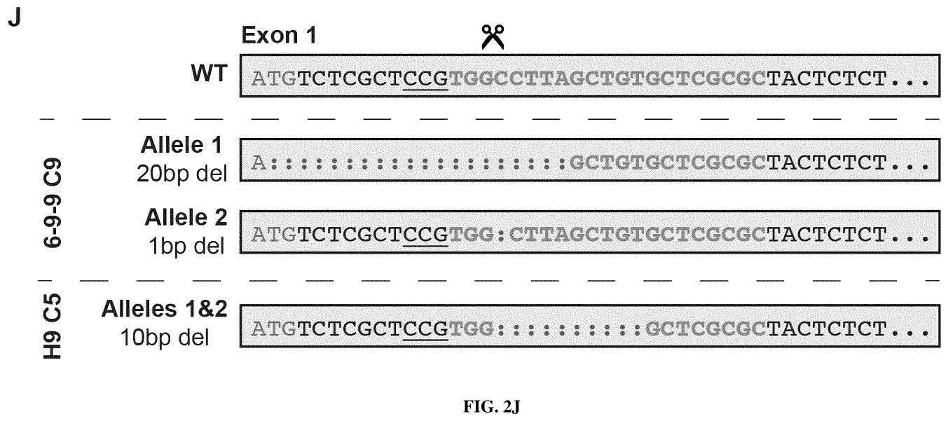

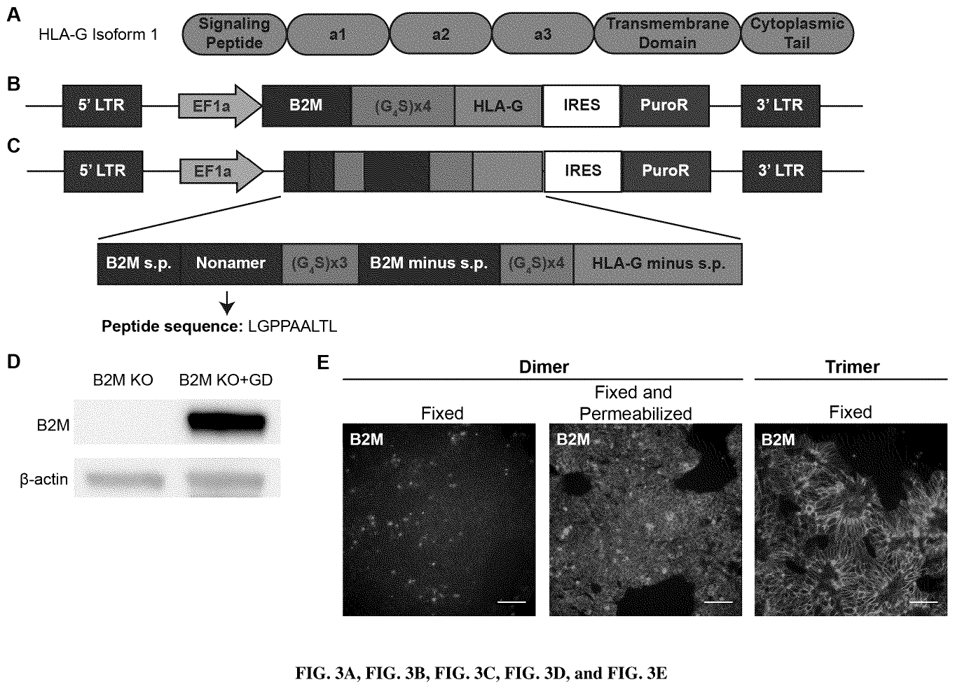

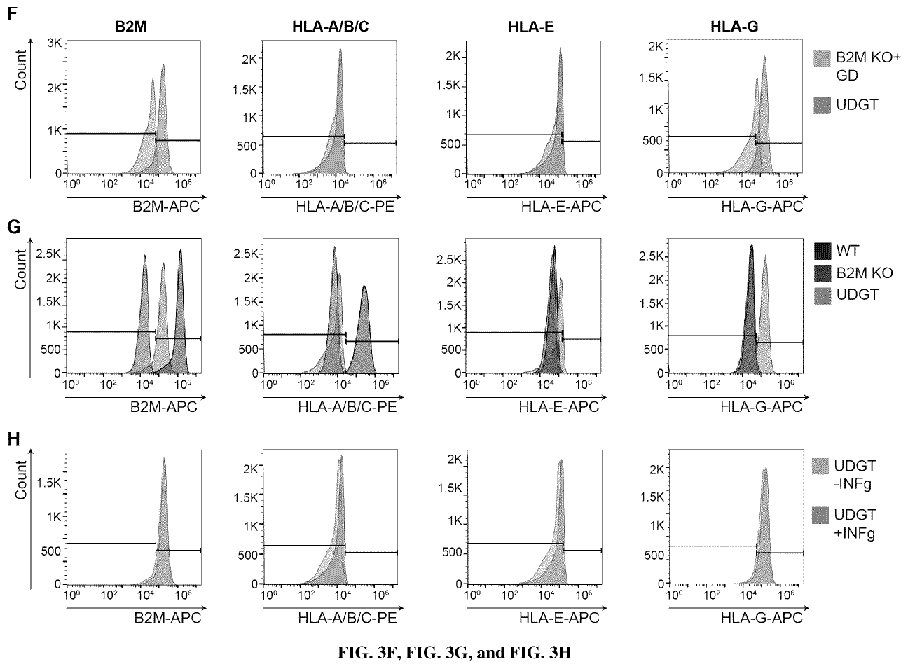

After generating B2M KO hPSCs, constructs were designed to obtain overexpression of HLA-G or HLA-E exclusively. HLA-G is unique in that it exists in 7 different isoforms. Of these, isoforms 1-4 are membrane bound while isoforms 5-7 are secreted. Additionally, only isoforms 1 and 5 have been shown to bind to B2M. As such, isoform 1 was selected for the fusion protein because it retains all three alpha domains, is membrane bound, complexes with B2M, and has been implicated in immune tolerance (FIG. 3A). Next experiment determined whether a synthetic dimer (B2M and HLAG) or trimer (B2M, HLAG, and peptide) design can be more efficacious in promoting surface expression. HLAs require the binding of a peptide for molecule stabilization and cell surface expression. While HLA-G is uniquely stable once expressed on the surface, owing to its shorter cytoplasmic tail domain, if it is unable to bind an appropriate peptide, surface expression cannot occur. HLA-G typically presents peptides derived from a variety of intracellular proteins; nevertheless, it is possible that, due to its tissue restricted expression pattern, peptides suitable for binding HLA-G are not present in all cell types. This can explain the sharp decrease in HLA-G expression observed previously, where upon hPSC differentiation to vascular smooth muscle cells, only 27% of them retained HLA-G expression. Alternatively, HLA-G dimer fusion is unable to form a complex with the ER protein TAP (transporter associated with antigen processing), a process on which peptide binding is dependent.

A dimer fusion protein consisting of B2M connected by a flexible non-cleavable linker to HLA-G was designed (FIG. 3B). For the trimer, an approach originally intended for the development of DNA vaccines was used, which has been effective at producing TAP independent assembly, stable surface expression, and uninhibited immune recognition with other MHC molecules. An HLA-G trimer that incorporated a peptide, B2M, and HLA-G was designed (FIG. 3C). The peptide sequence most commonly presented by HLA-G at the maternal-fetal interface was selected because of its contextual role in immune tolerance. These sequences were synthesized and cloned into a lentiviral plasmid backbone under the control of a constitutive promotor with a puromycin resistance gene. Cells were transduced with lentivirus containing this plasmid and purified by puromycin selection.

Western blot analysis of B2M, comparing our B2M KO cells and B2M KO cells with the HLA-G dimer (B2M KO+GD), indicated this construct was successfully incorporated and strongly expressed (FIG. 3D). However, using immunofluorescent microscopy only detected strong B2M expression intracellularly (FIG. 3E). This was not the case though with the B2M KO cells transduced with lentivirus containing the HLA-G trimer construct (Universal Donor HLA-G Trimer; UDGT), which showed strong surface expression of B2M (FIG. 3E). This indicates that the trimer construct does allow increased surface expression of engineered MHC molecules. This was further supported by quantification of expression by flow cytometry. It demonstrated higher surface expression of B2M and HLA-G in the UDGT cells as compared to the B2M KO+GD cells (FIG. 3F). To evaluate the efficacy of the engineered HLA expression profile, hPSCs were treated with interferon gamma to induce native HLA expression and compared wildtype (WT) and B2M KO cells with UDGT cells. WT cells showed expression of B2M and HLA-A/B/C without HLA-E or G, whereas B2M KO cells showed no B2M or HLA expression (FIG. 3G). UDGT cells only expressed B2M and HLA-G. This was independent of interferon gamma treatment (FIGS. 3G and 3H).

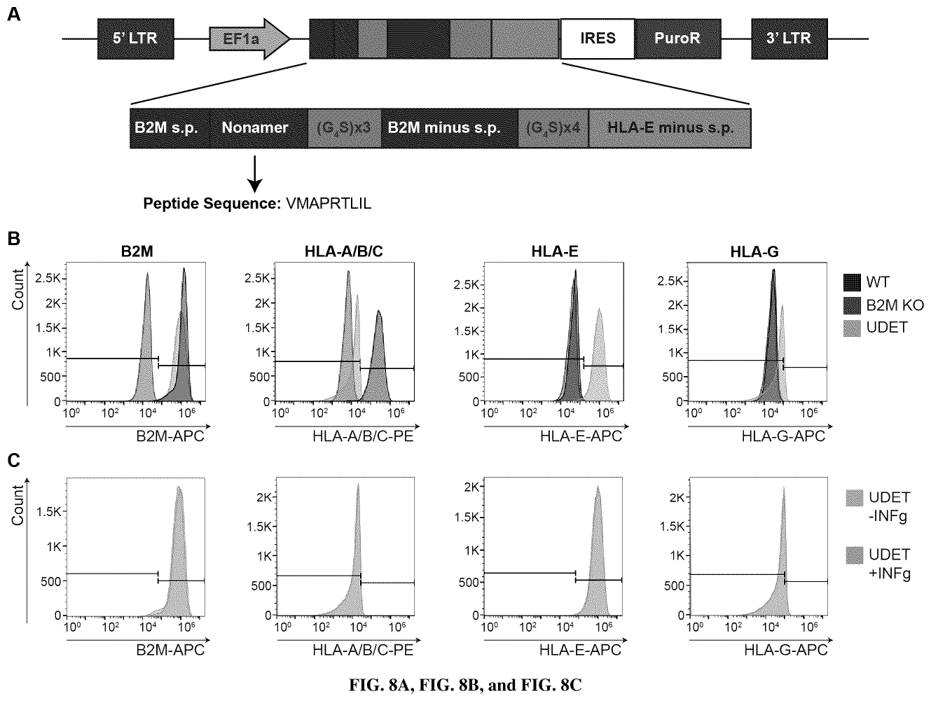

Upon confirming the HLA engineering strategy was successful with HLA-G, the same design principles were implemented to obtain functional HLA-E expression. The two common allelic variants of HLA-E differ only at position 107, where the amino acid at this location is either arginine or glycine. The glycine variant has increased thermal stability and cell surface expression, so this version was used in the fusion protein. HLA-E most often binds peptides processed from the signal peptide sequences of other class I HLAs. This includes HLA-A, B, C, and G with varying affinity. Meaning that HLA-E expression is reduced in the absence of other HLA molecule expression. Nonamer peptides derived from the signal peptide sequence of HLA-C and HLA-G induce strong CD94/NKG2A inhibitory immune interactions, but HLA-E presenting HLA-G derived peptide also triggers potent NK cell activation via CD94/NKG2C. Given this, a peptide derived from the HLA-C signaling peptide sequence was selected, which has no reported activating effects on NK cells (FIG. 8A). B2M KO cells were transduced with lentivirus containing the HLA-E trimer construct and selected for cells having successfully incorporated the construct. Analysis of HLA expression in interferon gamma treated hPSCs revealed these HLA-E trimer cells (Universal Donor HLA-E Trimer; UDET) strongly express B2M and HLA-E independent of interferon gamma, indicating that this design was successful (FIGS. 8B and 8C). Taken together, these data indicate that engineered HLA-E and HLA-G surface expression can be achieved in the absence of B2M by fusing B2M and a peptide to the HLA protein.

### Example 5. Genetically Stable Knockin of HLA Trimer Constructs

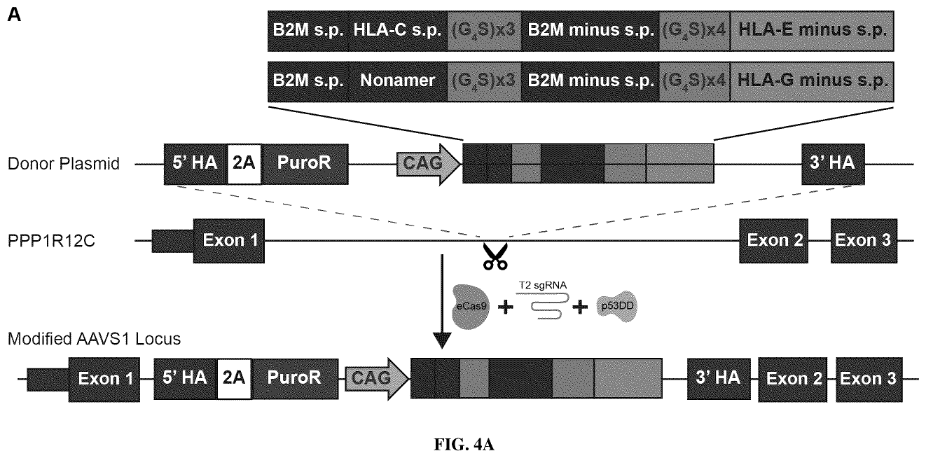

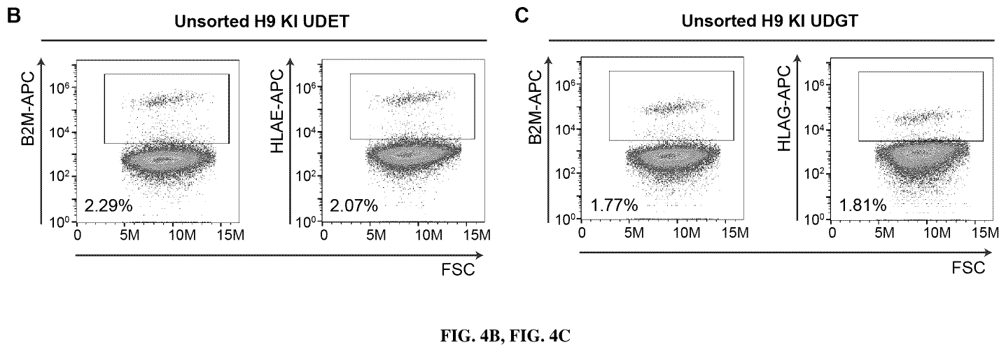

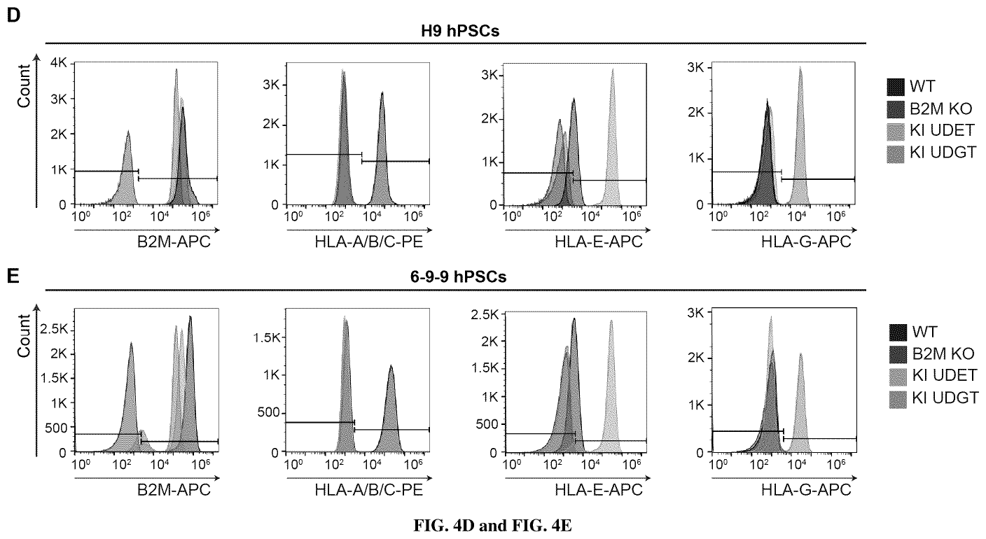

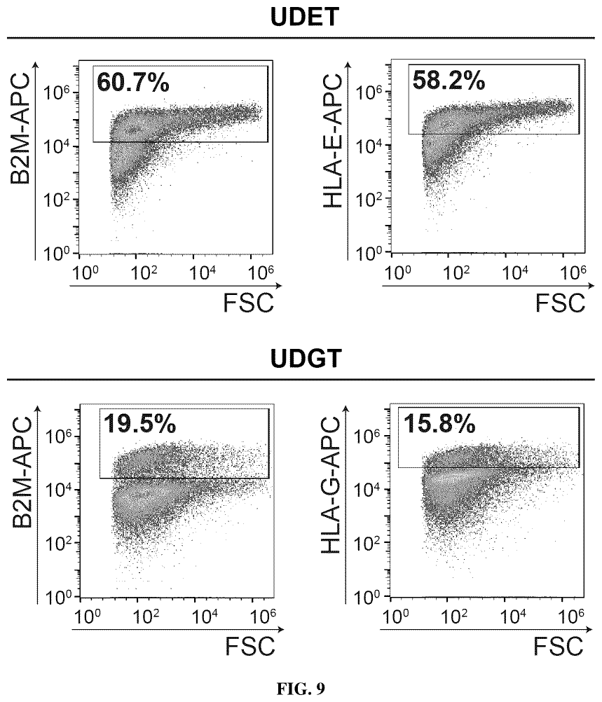

Next evaluated was whether the trimer constructs can be silenced during differentiation due to lentiviral integration. It was found that, in cardiac progenitor cells differentiated from UDET and UDGT hPSCs, the expression of HLA-E and G trimers was in fact decreased, indicative of silencing (FIG. 9). Of note, the constructs were not silenced at equal rates with the UDET cells remaining 60% positive for HLA-E, while UDGT cells showed less than 20% of the population still expressing HLA-G (FIG. 9). To address this, the integration method was altered for the trimer constructs and a CRISPR-Cas9 mediated safe harbor knockin at the AAVSI locus was generated. The trimer fusion constructs were cloned into donor plasmids and B2M KO cells were transfected with plasmids encoding eCas9, T2 sgRNA, the donor construct, and p53DD (FIG. 4A). Using this strategy, roughly 2% knockin efficiency was achieved (FIGS. 4B and 4C). The design of the donor construct permitted the expression of the puromycin resistance gene only if inserted immediately following an exon, rendering off target insertion rare and unlikely. Using puromycin, the successfully modified cells were purified and single cell colony selection was performed to identify successful heterozygous knockin clones.

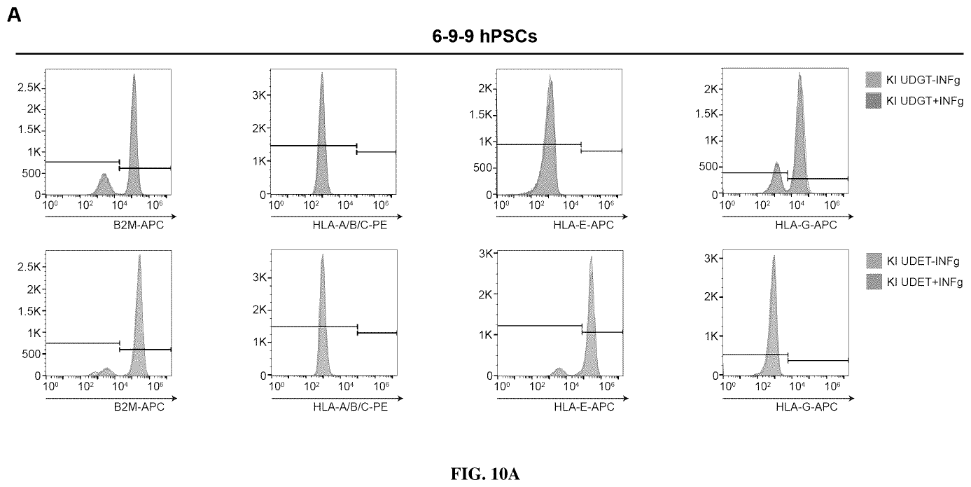

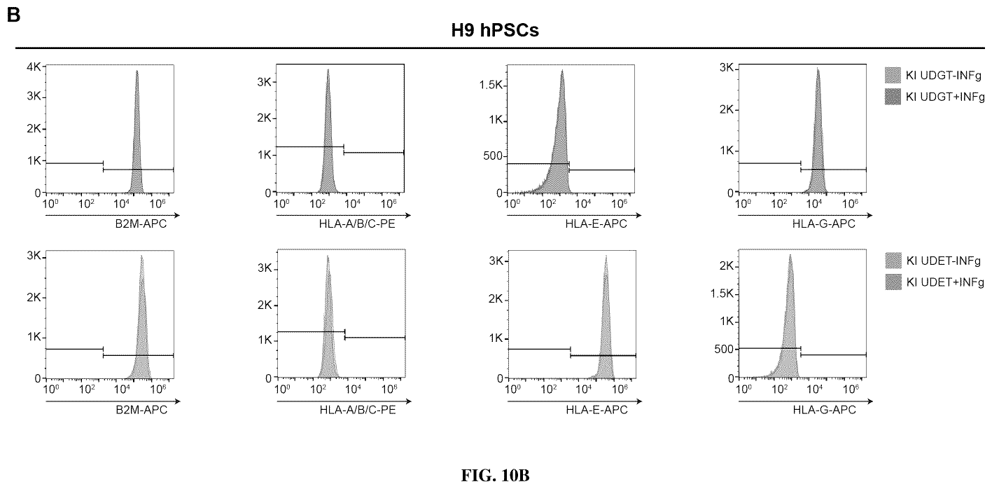

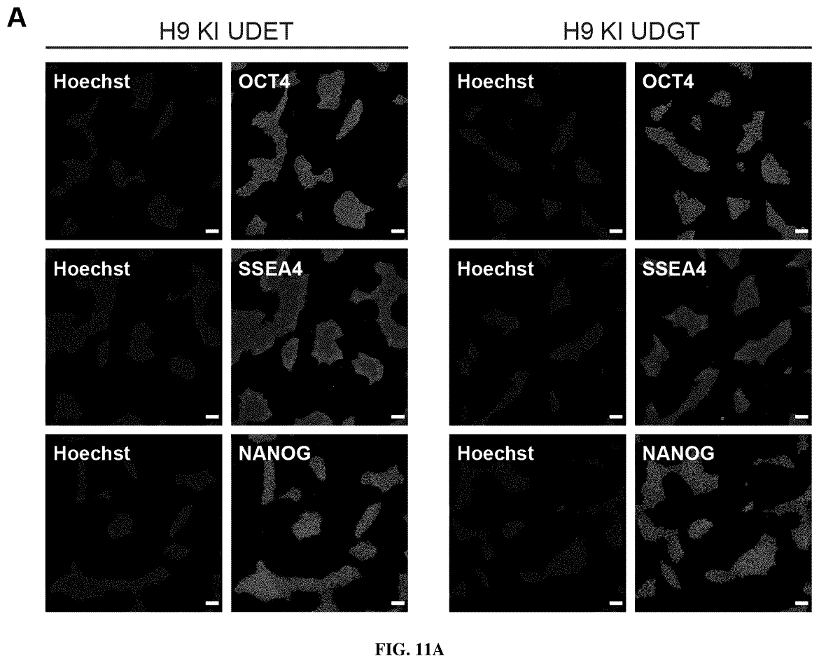

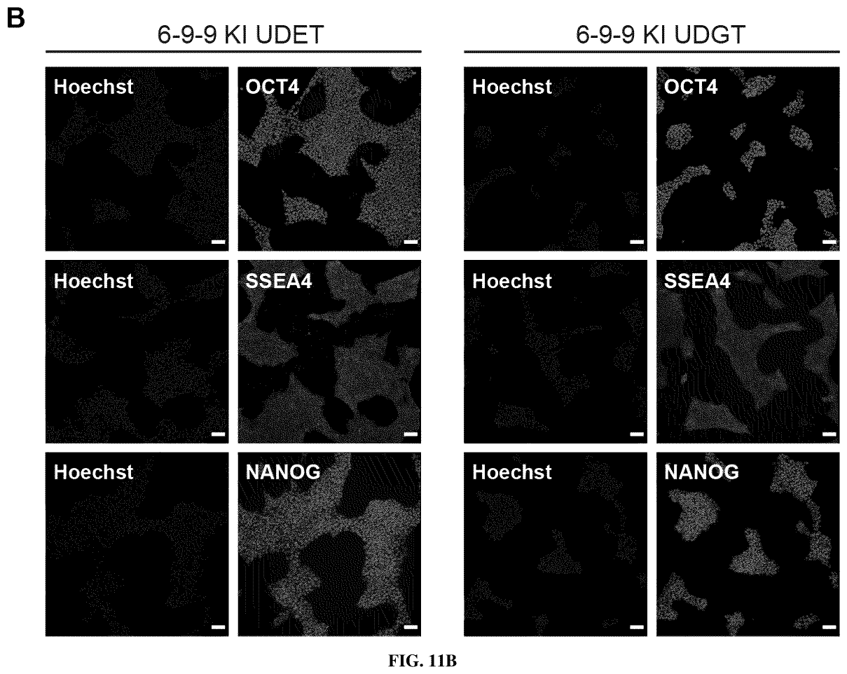

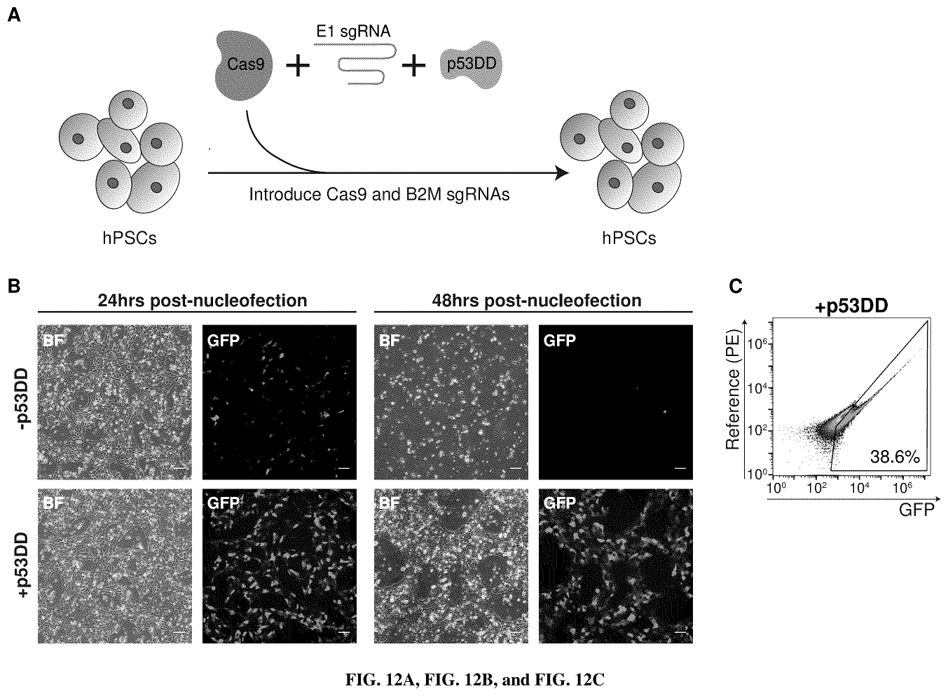

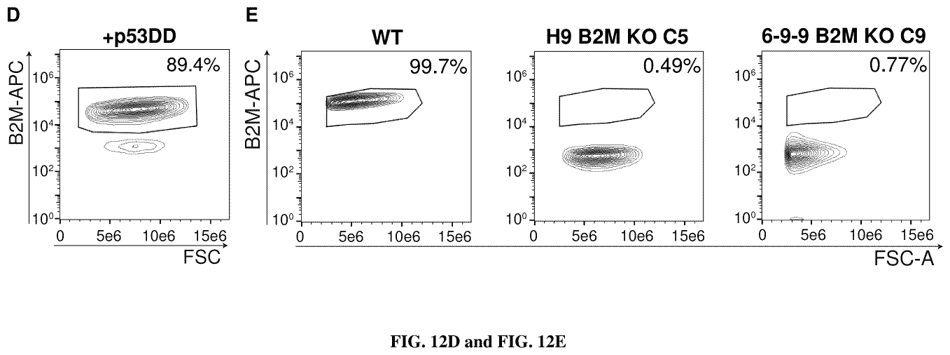

One single cell derived clone for each cell line (H9 and 6-9-9) and each candidate universal donor cell type (HLA-E and G trimers) was selected for further analysis. These cell lines are referred to as KI UDET and KI UDGT. Analysis of HLA gene expression was conducted for knockin cells and compared to WT and B2M KO cells (FIG. 4D). The KI UDGT and KI UDET cells showed engineered HLA expression that was independent from interferon gamma stimulation (FIGS. 4D and 4E, FIG. 10). Additionally, immunofluorescence analysis indicates that the cells remain pluripotent (FIG. 11). This data verifies the knockin design effectively introduced the trimer constructs to achieve stable engineered HLA expression.

### Example 6. In Vitro Analysis of Immune Rejection

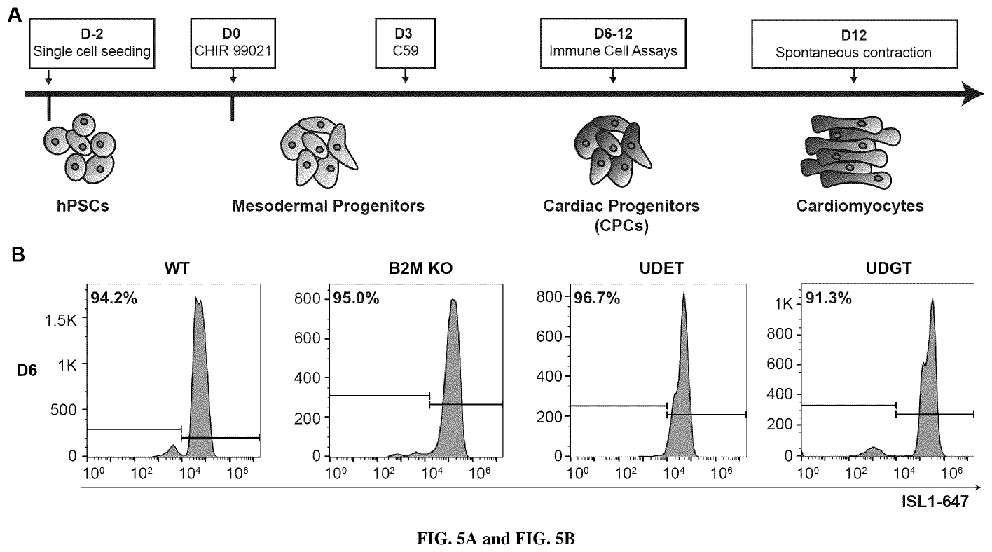

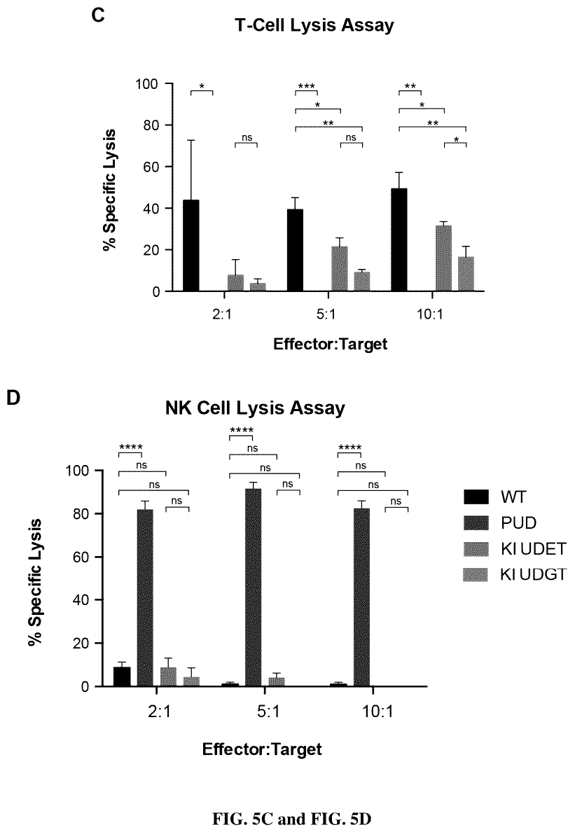

Having immunoengineered novel universal donor cells, their ability to evade immune rejection was tested in vitro. To do this, the cells were first differentiated to the committed CM progenitor state (FIG. 5A). It was verified that these modified cells can become cardiac progenitor cells (CPCs) by assaying for CPC marker ISL1, on day 6 of differentiation for WT, B2M KO, KI UDGT, and KI UDET (FIG. 5B). All of the modified cells readily differentiated with greater than 90% efficiency (FIG. 5B). Using the CPCs from each cell type, an in vitro luciferase release lysis assay was performed using human donor peripheral blood derived CD8+ T-cells. All of the target cell lines were transduced to constitutively express luciferase. After incubating the effector immune cells with our target CPC cells, WT CPCs were lysed across all effector to target cell ratios tested (FIG. 5C). The B2M KO cells were protected from T-cell mediated lysis, indicated by a significant decrease in the percent specific lysis to near zero. This protection was maintained with statistically significant decreases in lysis from the WT levels even in the presence of HLA-E or HLA-G trimer expression (FIG. 5C). While the KI UDGT cells showed slightly less lysis than KI UDET cells across all effector to target ratios. This difference was not enough to be considered statistically significant. This leads to the conclusion that HLA-E and HLA-G trimer expression do not elicit an immunogenic response from CD8+ T-cells in vitro.

Next, the ability of these universal donor cells to evade NK cell attack was tested. Accordingly, the same in vitro luciferase release lysis assay was performed with NK-92 cells as effector cells. NK-92 cells are an NK cell line expressing high levels of CD94/NKG2A, a common receptor involved in the NK cell immune response. An inverse lysis trend to that observed with T-cells for WT and B2M KO cells was observed. WT cells evaded NK cell detection and B2M KO cells were readily lysed (FIG. 5D). The introduction of these trimer constructs resulted in a significant reduction in NK cell mediated lysis compared to B2M KO cells, such that there was no significant difference between the KI UDET or KI UDGT cells and WT cells. (FIG. 5D). Again, a non-significant difference in lysis was observed between the KI UDET and KI UDGT cells, indicating that the engineered HLA expression protects the cells from an NK cell mediated immune response.

The ability to deliver on the promise of hPSCs for regenerative medicine and tissue engineering applications requires consideration of the immunological phenomena associated with transplantation. The idea of developing a universal donor cell source is not a new one; however, this study has demonstrated here a unique immunoengineering strategy to achieve this goal. We have successfully generated a Cas9 mediated B2M knockout and HLA trimer knockin resulting in the universal donor cells. Furthermore, the engineered HLA expression takes into consideration the unique ability of immune cell receptors and ligands to detect the peptide sequences presented by HLA molecules, and the potential causative role of these sequences in determining immune response. This study has also provided evidence that engineered HLA molecule expression favors the inclusion of an antigen peptide for strong surface expression. These findings are supported by previous studies that found HLA-E expression with a peptide can be used to reduce T-cell mediated lysis for retinal epithelial cells and blood progenitors derived from hPSCs in a class I negative background. A recent study demonstrated that exogenous expression of membrane HLA-G1 and soluble HLA-G5 in a B2M null hPSC line generates hypoimmunogenic differentiated cells. This study did not include an antigen peptide in their HLA molecule design which it was found here to be critical for robust surface expression of HLA-G. This contradictory report can be the result of differences in the genetic engineering approach. They inserted HLA-G directly to the B2M locus linking it to the endogenous B2M protein.

The universal donor cell immunogenicity was analyzed in vitro. It was demonstrated that these cells are protected from lysis by CD8+ T-cells, a primary effector cell of immune rejection, and NK cells, a secondary effector cell. While both versions of the universal donor cells effectively evade immune detection, the KI UDGT cells do show slightly reduced immunogenicity in direct comparison to the KI UDET cells. CD8+ T-cells and NK cells are not the only effector cells responsible for immune rejection. CD4+ T-cells also play a role in immune rejection; however, CD4+ T-cells respond to MHC class II molecules on antigen presenting cells. These cells are not expected to play a major role in myocardial rejection for several reasons. First, it has been shown CMs do not express class II HLAs required for CD4+ T-cell binding and recognition. Second, strong CD4+ activation is largely achieved by the recognition of class II HLAs on antigen presenting cells, such as dendritic cells. While this can be achieved through indirect allorecognition, the only class I HLA expressed on the universal donor cells, and therefore the only HLA available for capture, degradation and presentation by antigen presenting cells, is HLA-E or HLA-G. Both of which are minimally polymorphic and tolerance inducing. In addition, ILT-2 is expressed on dendritic cells, and its interaction with HLA-G has been shown to induce tolerance and prevent class II HLA expression. Therefore, strong CD4+ T-cell activation is not expected in response to the universal donor cells.

Another secondary effector cell in the immune rejection cascade is the B-cell. The design shown herein can account for B-cell activation in several different ways. First, the lack of CD4+ T-cell activation can mitigate B-cell activation and thus prevent secretion of antibodies and cytokines. Moreover, the binding of antibodies to a graft cell and stimulatory cytokine signals can recruit NK cells, which then detect and lyse the cells. That being said, the cells are immunoengineered specifically to inhibit NK cells either via CD94/NKG2 interactions with HLA-E or via ILT-2 and HLA-G. Furthermore, in the case of the KI UDGT cells, the HLA-G binding partner ILT-2 is expressed on B-cells and receptor ligand binding has been shown to prevent B-cell activation and antibody secretion.

One potential concern associated with this universal donor design is that the immune system can be less able to mount a response if the cells were infected by pathogens; however, these cells were not made completely immune privileged. The mechanisms of pathogen removal by cells of the innate immune system, such as neutrophils and macrophages, remain intact. In spite of this, a solution can be to incorporate a thymidine kinase kill switch into the cells as further insurance against infection, and this can be incorporated into this genetic engineering strategy. Overall, methods have been designed and provided for engineering HLA expression for universal donor stem cells aimed at cardiovascular regeneration. Uniquely, this method provides a direct comparison of two molecules long studied for their role in immune tolerance.

Additionally, this method results in robust engineered surface expression due to the inclusion of a peptide in the trimer constructs. Furthermore, this study has provided proof of principle studies illustrating the hypoimmunogenic properties of differentiated cardiac cells obtained from these universal donor stem cells. The universal donor cells can be used for transplantation medicine for cardiovascular disease and have broad applications to other cell types affected by degenerative or autoimmune diseases.

### Example 7. METHODS

Maintenance of hPSCs. Human pluripotent stem cells (H9 and 6-9-9) were maintained on either Matrigel (Corning) or iMatrix-511 silk (Stegment) coated plates in LaSR or mTeSR1 (Stemcell Technologies) pluripotent stem cell medium. hPSCs transduced with Luc2aNeo construct were maintained with 500 μg/mL geneticin. All drugs were removed upon initiating differentiation. Cells were routinely tested to ensure mycoplasma free culture conditions using an established PCR based detection method. The use of hESCs was approved by the Institutional Review Board of the Pennsylvania State University and the Embryonic Stem Cell Research Oversight Committee. All studies were conducted in accordance with the approved guidelines.

Maintenance of HEK 293 cells. Human embryonic kidney 293 cells were maintained in DMEM (Thermo Fisher Scientific) supplemented with 10% FBS (VWR) and passaged every three to four days via TrypLE Express (Thermo Fisher Scientific) mediated dissociation. Cells were routinely tested to ensure mycoplasma free culture conditions using an established PCR based detection method.

Maintenance of PB-derived CD8+ T-cells. These cells were maintained using ImmunoCult-XF T-cell Expansion Media (Stem Cell Technologies) and activated using ImmunoCult HuCD3/CD28/CD2 T-cell Activator (Stem Cell Technologies). The cells were cultured per the instructions provided by Stem Cell Technologies regarding the use of their media and activator. These cells were maintained for no longer than 21 days in culture.

Maintenance of NK-92 cells. These cells were cultured in MEM-alpha supplemented with 0.2 mM myo-inositol (Sigma), 0.1 mM 2-mercaptoethanol (Gibco), 0.02 mM folic acid (Sigma), 100 IU/mL IL-2 (Peprotech), 12.5% FBS, 12.5% horse serum (Thermo Fisher Scientific). Fresh media was added every 2-3 days, and cells were gently pipetted to disperse clusters. Cell density was monitored to ensure it remained between le5-le6 cells/mL at all times. Approximately every 5 days the cells were passaged by collection, centrifugation and resuspension in fresh media. Cells were routinely tested to ensure mycoplasma free culture conditions using an established PCR based detection method.

Germ layer differentiation. For ectoderm differentiation: Cells were plated at low density (20% confluent). On day 0 and every day after, media was changed with LaSR Basal media for up to 6 days. For mesoderm differentiation: Cells were cultured until 80% confluent. On day 0,media was changed with RPMI (Thermo Fisher Scientific) supplemented with 100 μg/mL L-ascorbic acid and 6 μM CHIR99021. On day 1 media was changed to RPMI supplemented with 200 μg/mL L-ascorbic acid and 0.5% HSA (Biological Industries, 10% solution). Cells were collected on day 1 or 2.

Generation of HVPs and CMs. Cardiac differentiation of hPSCs was initiated when hPSCs seeded on Matrigel coated plates reached 80% confluence. Differentiation was performed according to previously published GiWi method. Briefly, at day 0, cells were treated RPMI supplemented with 100 μg/mL L-ascorbic acid and 6 μM CHIR99021, followed by a change with RPMI supplemented with 200 μg/mL L-ascorbic acid and 0.5% HSA medium on day 1 and 2. On day 3, 2 μM Wnt-C59 (Tocris) was added, followed by a medium change with RPMI supplemented with 200 μg/mL L-ascorbic acid and 0.5% HSA on day 5. On day 6 and every 3days after, cells were then cultured in RPMI supplemented with 200 μg/mL L-ascorbic acid, 0.25% HSA, selenium, transferrin, and insulin. Cells were considered cardiac progenitors between day 8 and day 12 and cardiomyocytes following day 12.

Generation of B2M KO sgRNA plasmid constructs. Two gRNAs were designed, one targeted to exon 1 (CGCGAGCACAGCTAAGGCCA, SEQ ID NO: 37) and one targeted to exon 2 (TGTGAACCATGTGACTTTGTC, SEQ ID NO: 37). These gRNAs were ligated into a plasmid backbone, pGuide (This plasmid was a gift from Kiran Musunuru. Addgene #64711), that was linearized with BbsI (New England Biolabs). Sanger sequencing was used to confirm successful incorporation of the gRNA sequence. These plasmids are available as pLR05_pGuide_B2M_sgRNA1 and pLR06_pGuide_B2M_sgRNA2 from Addgene. The plasmid backbone for B2M sgRNA insertion was linearized using the BbsI (New England Biolabs) restriction enzyme. The linearization reaction was performed by incubating the reaction at 55° C. (BsmBI) or 37° C. (BbsI) for 5 hours and purifying the linearized plasmid from an agarose gel following electrophoresis using a Zymoclean Gel DNA Recovery Kit (Zymo Research, D4001). The forward and reverse SOX17 sgRNA DNA oligos (synthesized by IDT, Appendix B Table 6) were annealed and ligated into the linearized backbone. The ligated plasmid was transformed into Stb13 competent E.coli (Thermo Fisher Scientific) and streaked on an Ampicillin containing agar plate (Thermo Fisher Scientific). A liquid culture was inoculated for 4-8 E.coli colonies and allowed to culture overnight. Plasmid was extracted from the E.coli using the Zyppy Plasmid Miniprep Kit (Zymo Research, D4020), and Sanger Sequencing was performed to identify a successfully ligated plasmid containing the sgRNA sequence. Sanger Sequencing was performed by the Penn State Huck Institutes Genomics Core Facility staff.

Lipofection of 293 cells. Lipofectamine reagent (Thermo Fisher Scientific) was combined with P3000 reagent (Thermo Fisher Scientific) and 3 μg total of the desired plasmid DNA in basal media. This solution was then added to cells. Media was changed the next day, washing the cells once with culture media to remove the lipofectamine reagents. All plasmid DNA used was prepared using the Invitrogen PureLink HiPure Plasmid Filter Midiprep Kit.

Generation of B2M KO cells. Cells were dissociated with Accutase for 10 minutes at 37° C. and pelleted. The cell pellet was resuspended in P3 Solution (Lonza) with 16 μg of plasmid DNA, including 7 μg CAG-Cas9-T2A-EGFP-ires-puro (This plasmid was a gift from Timo Otonkoski. Addgene #78311), 7 μg pLR05_pGuide_B2M_sgRNA1, and 2 μg pCE-mp53DD (This plasmid was a gift from Shinya Yamanaka. Addgene #41856). The mixture was transferred to a cuvette and nucleofected using the CB 150 program on the Lonza 4D Nucleofector. All plasmid DNA used was prepared using an Invitrogen PureLink HiPure Plasmid Filter Midiprep Kit. Cells were plated at a high density with 5 μM Y27632. The next day media was changed and for 1-2 days following 1 μg/mL puromycin was added to the media to select for cells that were successfully nucleofected. Cells were then plated at a single cell density and single cell derived colonies were picked and expanded. Single cell derived colonies were tested via flow cytometry to identify successful homozygous KO clones. These clones were then further characterized, and allele sequencing was performed using PCR amplified DNA of the cut site and the TOPO TA Cloning Kit (Thermo Fisher Scientific) following the manufacturer's instructions.

Generation of lentiviruses. To generate lentiviruses, the cargo plasmid and 2nd generation packaging plasmids psPAX2 and pMD2.G were added into OptiMEM (Thermo Fisher Scientific) and incubated at room temperature for 5 minutes. psPAX2 and pMD2.G were gifts from Didier Trono (Addgene plasmid #12260 and 12259). Fugene HD reagent (Promega) was added mixed by careful pipetting before incubating at room temperature for 10-15 minutes. All plasmid DNA used was prepared using an Invitrogen PureLink HiPure Plasmid Filter Midiprep Kit (Thermo Fisher Scientific). This solution was then added into pre-warmed DMEM+10% FBS to make the transfection media. Media was changed on 90% confluent HEK 293 cells using 0.5X the normal culture volume of transfection media. Cells were incubated overnight at 37° C. and media was changed the following morning with pre-warmed DMEM with 10% FBS using 1.5X the normal culture volume. 24 hrs, 48 hrs, and 72 hrs after this media change, virus containing media was collected from the cells and the media was changed with pre-warmed DMEM with 10% FBS using 1.5X the normal culture volume. The cells were disposed of after the third collection.

The collected virus containing media was stored at 4C until collection is complete. Virus containing media was centrifuged for 5 minutes at 1.5 g and the supernatant was collected. Concentration was done using Lenti-X Concentrator (TaKaRa Bio USA) according to the manufacturer's instructions. Concentrated virus was resuspended in DMEM and stored in single use aliquots at −80° C. until used.

Lentiviral infection of HEK 293 cells. To infect HEK 293 cells, 200μL of fresh virus containing media is added directly into the culture media of those cells and gently pipetted to mix.

Lentiviral infection of hPSCs cells. To infect hPSCs, an 80% confluent well of hPSCs was washed with DPBS and dissociated using Accutase for 5 minutes and pelleted. The supernatant was aspirated, and the cell pellet was resuspended in 50% culture media and 50% concentrated virus solution with a total volume of 100 μL. Cells were incubated at 37° C. for 30minutes and then plated in prewarmed culture media with 5 μM Y27632 and incubated overnight. Media was changed 24 hrs later.

Generation of knockin UDET and UDGT hPSCs. The dimer fusion proteins were designed by joining the B2M CDS sequence to the HLA-E or HLA-G CDS sequence by a flexible non cleavable linker (GGGGS)×3. The trimer fusion protein design was guided by previous research. The signaling peptide sequence for B2M was followed by a nonamer peptide sequence for HLA binding and presentation. This was then linked to the remaining B2M protein sequence by a flexible linker (GGGGS)×3 which was in turned linked to the sequence for HLAE or HLAG by a flexible linker (GGGGS)×4. The signaling peptide portion of the HLAE or HLAG coding sequence was not included. For all fusion proteins, a point mutation (A267G) was made to the B2M sequence to remove a restriction site but maintain the amino acid sequence. The Kozak sequence was included at the start of each fusion protein. These fusion proteins were synthesized by Genewiz and cloned into pXL001 (This plasmid was a gift from Sean Palecek. Addgene #26122) using restriction enzymes SpeI and EcoRI (New England Biolabs). These plasmids are available as pLR19_pXL001_CSIG-B2M-HLAE and pLR20_pXL001_PLASIG-2M-HLAG from Addgene.

To generate the knockin donor plasmids, the trimer fusion coding sequence was PCR amplified from pLR19_pXL001_CSIG-B2M-HLAE and pLR20_pXL001_PLASIG-B2 M-HLAG using the Q5 High-Fidelity 2X Master Mix (New England Biolabs) and purified following gel electrophoresis. This sequence was ligated into AAVS1-Pur-CAG-EGFP (This plasmid was a gift from Su-Chun Zhang. Addgene #80945), which was linearized using restriction enzymes SalI and MluI (New England Biolabs), using the NEBuilder HiFi DNA Assembly Master Mix (New England Biolabs). These plasmids are available as AAVSI_KIDonor_CSIG-B2M-HLAE or AAVSI_KIDonor_PLASIG-B2M-HLAG from Addgene.

To generate the knockin cells B2M KO cells were dissociated with Accutase for 10 minutes at 37° C. and pelleted. The cell pellet was resuspended in P3 Solution (Lonza) with 16 μg of plasmid DNA, including 7 μg eCas9-T2gRNA, 7 μg donor plasmid (either AAVS1_KIDonor_CSIG-B2M-HLAE or AAVSI_KIDonor_PLASIG-B2M-HLAG), and 2 μg pCE-mp53DD. The T2 sgRNA (GGGGGCCACTAGGGACAGGAT) was cloned into the eSpCas9 (1.1) plasmid (This was a gift from Feng Zhang. Addgene #71814) which was linearized with restriction enzyme BbsI (New England Biolabs). All plasmid DNA used was prepared using an Invitrogen PureLink HiPure Plasmid Filter Midiprep Kit. The mixture was transferred to a cuvette and nucleofected using the CB150 program on the Lonza 4D Nucleofector. Cells were plated at a high density with 5 μM Y27632. Following puromycin selection to purify the successfully modified cells, single cell colony selection was performed, and PCR based genotyping was used to identify heterozygous knockin clones.

RNA sequencing. Total RNA was isolated with Direct-Zol RNA Kits (Zymo Research, USA). RNA quality was checked using the BioAnlayzer 2100. Library preparation type used was Illumina TrueSeq Stranded mRNA, Poly-A selection. Samples were sequenced on an Illumina HiSeq2500. Briefly, the FASTQ sequence files were mapped to Human Genome, GRCh37 with STAR aligher. The outputted BAM alignment files were used for further analysis. The expression count table was produced by inputting the BAM files produced in a previous step and annotation file gencode.v27lift37 from Gencode into the FeatureCounts program.

Flow cytometry analysis. For staining and analysis of fixed cells, after dissociation with TrypLE Express (differentiated cells) or Accutase (hPSCs), cells were pelleted and resuspended in DPBS with 1% formaldehyde for 30 minutes at room temperature. Cells were pelleted and washed 3 times with DPBS. Cells were stained with primary and secondary antibodies in DPBS with 0.1% Triton X-100 and 0.5% BSA for 2 hours at room temperature. Cells were pelleted and washed 3 times with DPBS with 0.5% BSA.

For staining and analysis of live cells, cells were dissociated with TrypLE Express (differentiated cells) or Accutase (hPSCs) and pelleted. For suspension cultures of hematopoietic or lymphoid progenitors, cells were filtered with a 100 μm cell strainer and pelleted. Cells were then resuspended in DPBS with 0.5% BSA and the appropriate conjugated primary antibody dilution and incubated at room temperature for 30 minutes. Cells were pelleted and washed with DPBS with 0.5% BSA.

Data were collected on a BD Accuri C6 Plus flow cytometer and analyzed using FlowJo. Gating was based on the corresponding untreated or secondary antibody stained cell control.

Immunostaining. Cells were fixed with 4% formaldehyde (Sigma) for 15 min at room temperature. Cells were washed 3 times with PBS and then blocked for 1 hour at room temperature with DPBS with 0.4% Triton X-100 and 5% non-fat dry milk (BioRad). Cells were stained with primary and secondary antibodies in DPBS with 0.4% Triton X-100 and 5% non-fat dry milk. Nuclei were stained with Hoechst 33342 (Thermo Fisher Scientific). A Nikon TI Eclipse epifluorescence microscope was used for image capture and analysis. Fiji and custom Matlab scripts were used for further analysis and quantification.

Western blotting. Cells were washed with DPBS and lysed with Mammalian Protein Extraction Reagent (Thermo Fisher) with 1X Halt's Protease and Phosphatase (Thermo Fisher) by incubation for 3 minutes. Cell lysate was collected and stored at −80° C. until used. Samples were mixed with Laemmli sample buffer (BioRad) at a working concentration of 1X and incubated at 97 C for 5 minutes. Samples were loaded into a pre-cast MP TGX stain free gel (BioRad) and run at 200V for 30 minutes in 1X Tris/Glycine/SDS buffer (BioRad). Protein was transferred to a PVDF membrane using a Trans-blot Turbo Transfer System (BioRad). The membrane was blocked for 30 minutes at room temperature in 1X TBST with 5% Dry Milk. The membrane was incubated overnight at 4° C. with primary antibodies and for 1 hour at room temperature with secondary antibodies in 1X TBST with 5% Dry Milk. The membrane was washed between each antibody exposure with 1X TBST. Chemiluminescence was activated using Clarity Western ECL Substrate (BioRad) and the blot was imaged using a ChemiDoc Touch Imaging System and Image Lab software (BioRad). Blots were analyzed using Fiji software.

Luciferase release assay. Cardiac cells were counted and dispersed evenly into 96 well plates and allowed to adhere for at least 72 hours prior to initiating a lysis assay. At least one media change was done between cardiac cell plating and assay initiation. Just prior to the start of the assay, at least three wells dispersed across the plate were dissociated and counted to ensure even density was achieved. The cell counts for these wells were then averaged and to obtain a target cell number. Based on this, the number of effector cells required for 10:1, 5:1, and 2:1 effector to target ratios was determined. The luminescence was then measured to establish a baseline. Media was changed and 100 μL of effector cell media containing 150 μg/mL D-luciferin (Promega) was added. Cells were incubated for 5 minutes before luminescence was measured for the 0 hour time point. Effector cells were collected, pelleted and dispersed into wells (at least 3 wells for each ratio). Cells were then incubated together at 37 C for 2 hrs. After 2 hours, 150 μg/mL D-luciferin was added and cells were incubated for 5 minutes prior to obtaining the 2 hour luminescence measurement. Effector cells were resuspended in their normal culture media prior to dispersal for the assay. Cardiac cells in effector medium with no effector cells were used for a spontaneous death control. Cardiac cells in effector medium with 0.1% Triton-X (Sigma) were used as a positive control. All luminescence measurements were collected with a Tecan Infinite M Plex plate reader. Effector cells were collected, pelleted and dispersed into wells (at least 3 wells for each ratio). The percent of specific lysis was calculated using previously established methods.

Statistics. Experiments were performed in triplicate. Data obtained from multiple experiments or replicates are shown as the mean±standard error of the mean. Where appropriate, Student's t test or a two-way ANOVA was utilized (alpha=0.05) with a Bonferroni or Tukey's post hoc test. Data were considered significant when p<0.05. Statistical tests were performed using GraphPad Prism

### REFERENCES

1. Abbas, A. K., Lichtman, A. H. & Pillai, S. Transplantation Immunology, in Cellular and Molecular Immunology 373-396 (Elsevier, 2018). doi: 10.1016/B978-1-4377-1604-7.00048-8.

2. Bentley, T. S., Phillips, S. J. & Hanson, S. G. Milliman Research Report: 2017 U.S. organ and tissue transplant cost estimates and discussion. (2017).

3. Lian, X. et al. Robust cardiomyocyte differentiation from human pluripotent stem cells via temporal modulation of canonical Wnt signaling. Proc. Natl. Acad. Sci. 109, E1848 -E1857 (2012).

4. Lian, X. et al. Directed cardiomyocyte differentiation from human pluripotent stem cells by modulating Wnt/β-catenin signaling under fully defined conditions. Nat. Protoc. 8, 162-75 (2013).

5. Lian, X. et al. Chemically defined, albumin-free human cardiomyocyte generation. Nat. Methods 12, 595-596 (2015).

6. Araki, R. et al. Negligible immunogenicity of terminally differentiated cells derived from induced pluripotent. Nature 494, 100-104 (2013).

7. Han, X. et al. Generation of hypoimmunogenic human pluripotent stem cells. 116, (2019).

8. Xu, H. et al. Targeted Disruption of HLA Genes via CRISPR-Cas9 Generates iPSCs with Enhanced Immune Article Targeted Disruption of HLA Genes via CRISPR-Cas9Generates iPSCs with Enhanced Immune Compatibility. Stem Cell 24, 566-578.e7 (2019).

9. Deuse, T. et al. Hypoimmunogenic derivatives of induced pluripotent stem cells evade immune rejection in fully immunocompetent allogeneic recipients. Nat. Biotechnol. 37, (2019).

10. Gornalusse, G. G. et al. HLA-E-expressing pluripotent stem cells escape allogeneic responses and lysis by NK cells. Nat. Publ. Gr. 35, 765-772 (2017).

11. Liao, N., Bix, M., Zijlstra, M., Jaenisch, R. & Raulet, D. MHC Class I Deficiency: Susceptibility to Natural Killer (NK) Cells and Impaired NK Activity. Science (80-.). 253, 199-202 (1991).

12. Bix, M. et al. Rejection of class I MHC-deficient haemopoietic cells by irradiated MHC-matched mice. Nature 349, 329-331 (1991).

13. Rong, Z. et al. An effective approach to prevent immune rejection of human ESC-derived allografts. Cell Stem Cell 14, 121-130 (2014).

14. Kawamura, T. et al. Cardiomyocytes Derived from MHC-Homozygous Induced Pluripotent Stem Cells Exhibit Reduced Allogeneic Immunogenicity in MHC-Matched Non-human Primates. Stem Cell Reports 6, 312-320 (2016).

15. Shiba, Y. et al. Allogeneic transplantation of iPS cell-derived cardiomyocytes regenerates primate hearts. Nature 538, 388-391 (2016).

16. Strong, R. K. et al. HLA-E allelic variants: Correlating differential expression, peptide affinities, crystal structures, and thermal stabilities. J. Biol. Chem. 278, 5082-5090 (2003).

17. Rouas-Freiss, N., Marchal, R. E., Kirszenbaum, M., Dausset, J. & Carosella, E. D. The al domain of HLA-G1 and HLA-G2 inhibits cytotoxicity induced by natural killer cells: Is HLA-G the public ligand for natural killer cell inhibitory receptors? Proc. Natl. Acad. Sci. U.S.A. 94, 5249-5254 (1997).

18. Naji, A. et al. Binding of HLA-G to ITIM-Bearing Ig-like Transcript 2 Receptor Suppresses B Cell Responses. J. Immunol. 192, 1536-1546 (2014).

19. Pazmany, L. et al. Protection from natural killer cell-mediated lysis by HLA-G expression on target cells. Science (80-.). 274, 792-795 (1996).

20. Dumont, C. et al. CD8PPD-1-ILT2p T cells are an intratumoral cytotoxic population selectively inhibited by the immune-checkpoint HLA-G. Cancer Immunol. Res. 7, 1619-1632 (2019).

21. Clements, C. S. et al. Crystal structure of HLA-G: A nonclassical MHC class I molecule expressed at the fetal-maternal interface. Proc. Natl. Acad. Sci. U.S.A. 102, 3360-3365 (2005).

22. Ferreira, L. M. R., Meissner, T. B., Tilburgs, T. & Strominger, J. L. HLA-G: At the Interface of Maternal-Fetal Tolerance. Trends Immunol. 38, 272-286 (2017).

23. Carosella, E. D., Rouas-freiss, N., Roux, D. T., Moreau, P. & Lemaoult, J. HLA-G: An Immune Checkpoint Molecule. Advances in Immunology vol. 127 (Elsevier Inc., 2015).

24. Lila, N. et al. Implication of HLA-G molecule in heart-graft acceptance. Lancet 355, 2138 (2000).

25. Llano, M. et al. HLA-E-bound peptides influence recognition by inhibitory and triggering CD94/NKG2 receptors: Preferential response to an HLA-G-derived nonamer. Eur. J. Immunol. 28, 2854-2863 (1998).

26. Andre, P. et al. Anti-NKG2A mAb Is a Checkpoint Inhibitor that Promotes Anti-tumor Immunity by Unleashing Both T Article Anti-NKG2A mAb Is a Checkpoint Inhibitor that Promotes Anti-tumor Immunity by Unleashing Both T and NK Cells. Cell 175, 1731-1743 (2018).

27. Ishitani, A. et al. Protein Expression and Peptide Binding Suggest Unique and Interacting Functional Roles for HLA-E, F, and G in Maternal-Placental Immune Recognition. J. Immunol. 171, 1376-1384 (2003).

28. Pereira, B. I. et al. Senescent cells evade immune clearance via HLA-E-mediated NK and CD8+ T cell inhibition. Nat. Commun. 10, (2019).

29. Crew, M. D., Cannon, M. J., Phanavanh, B. & Garcia-Borges, C. N. An HLA-E single chain trimer inhibits human NK cell reactivity towards porcine cells. Mol. Immunol. 42, 1205-1214 (2005).

30. Haapaniemi, E., Botla, S., Persson, J., Schmierer, B. & Taipale, J. CRISPR-Cas9 genome editing induces a p53-mediated DNA damage response. Nat. Med. (2018) doi: 10.1038/s41591-018-0049-z.

31. Ihry, R. J. et al. p53 inhibits CRISPR-Cas9 engineering in human pluripotent stem cells. Nat. Med. 1-8 (2018) doi: 10.1038/s41591-018-0050-6.

32. Abbas, A. K., Lichtman, A. H. & Pillai, S. Antigen presentation to T lymphocytes and the functions of MHC molecules, in Cellular and molecular immunology 117-140 (Elsevier, 2018).

33. Davis, D. M. et al. Impaired spontaneous endocytosis of HLA-G. Eur. J. Immunol. 27, 2714-2719 (1997).

34. Hansen, T., Yu, Y. Y. L. & Fremont, D. H. Preparation of stable single-chain trimers engineered with peptide, B2 microglobulin, and MHC heavy chain. Curr. Protoc. Immunol. (2009) doi: 10.1002/0471142735.im1705s87.

35. Yu, Y. Y. L., Netuschil, N., Lybarger, L., Connolly, J. M. & Hansen, T. H. Cutting Edge: Single-Chain Trimers of MHC Class I Molecules Form Stable Structures That Potently Stimulate Antigen-Specific T Cells and B Cells. J. Immunol. 168, 3145-3149 (2002).

36. Lee, N., Goodlett, D. R., Ishitani, A., Marquardt, H. & Geraghty, D. E. HLA-E surface expression depends on binding of TAP-dependent peptides derived from certain HLA class I signal sequences. J. Immunol. 160, 4951-60 (1998).

37. Karimi, M. A. et al. Measuring cytotoxicity by bioluminescence imaging outperforms the standard chromium-51 release assay. PLOS One 9, (2014).

38. Shi, L. et al. Generation of hypoimmunogenic human pluripotent stem cells via expression of membrane-bound and secreted β2m-HLA-G fusion proteins. Stem Cells (2020) doi: 10.1002/stem.3269.

39. Greco, R. et al. Improving the safety of cell therapy with the TK-suicide gene. Front. Pharmacol. 6, 1-13 (2015).

40. Lian, X. et al. A Small Molecule Inhibitor of Src Family Kinases Promotes Simple Epithelial Differentiation of Human Pluripotent Stem Cells. PLOS One 8, (2013).

41. Bao, X. et al. Chemically-defined albumin-free differentiation of human pluripotent stem cells to endothelial progenitor cells. Stem Cell Res. 15, 122-129 (2015).

42. Young, L., Sung, J., Stacey, G. & Masters, J. R. Detection of Mycoplasma in cell cultures. Nat. Protoc. 5, 929-934 (2010).

SEQUENCES
SEQ ID NO: 1, B2M signaling peptide (HLA-E trimer)
ATGTCTCGCTCCGTGGCCTTAGCTGTGCTCGCGCTACTCTCTCTTTCTGGCCTCGAGG
CT
SEQ ID NO: 2, B2M signaling peptide (HLA-E trimer)
MSRSVALAVLALLSLSGLEA
SEQ ID NO: 3, B2M minus signaling peptide (HLA-E trimer)
ATCCAGCGTACGCCAAAGATTCAGGTTTACTCACGTCATCCAGCAGAGAATGGAAA
GTCAAATTTCCTGAATTGCTATGTGTCTGGGTTTCATCCATCCGACATTGAAGTTGAC
TTACTGAAGAATGGAGAGAGAATTGAAAAAGTGGAGCATTCAGACTTGTCTTTCAG
CAAGGACTGGTCTTTCTATCTCTTGTACTACACTGAGTTCACCCCCACTGAAAAAGA
TGAGTATGCCTGCCGTGTGAACCATGTGACTTTGTCACAGCCCAAGATAGTTAAGTG
GGATCGCGACATG
SEQ ID NO: 4, B2M minus signaling peptide (HLA-E trimer)
IQRTPKIQVYSRHPAENGKSNFLNCYVSGFHPSDIEVDLLKNGERIEKVEHSDLSFSKDW
SFYLLYYTEFTPTEKDEYACRVNHVTLSQPKIVKWDRDM
SEQ ID NO: 5, HLA-C signaling peptide (HLA-E trimer)
GTTATGGCTCCGCGGACTTTAATTTTA
SEQ ID NO: 6, HLA-C signaling peptide (HLA-E trimer)
VMAPRTLIL
SEQ ID NO: 7, Linker (HLA-E trimer)
GGTGGTGGCGGATCCGGTGGTGGCGGTTCTGGTGGTGGCGGCTCC
SEQ ID NO: 8, Linker (HLA-E trimer)
GGGGSGGGGSGGGGS
SEQ ID NO: 9, Linker (HLA-G trimer)
GGTGGTGGCGGTTCTGGTGGTGGCGGTAGTGGCGGCGGAGGAAGCGGTGGTGGCGG
TTCC
SEQ ID NO: 10, Linker (HLA-G trimer)
GGGGSGGGGSGGGGSGGGGS
SEQ ID NO: 11, HLA-E (HLA-E trimer minus signaling peptide)
GGATCTCACTCCTTGAAGTATTTCCACACTTCCGTGTCCCGGCCCGGCCGCGGGGAG
CCCCGCTTCATCTCTGTGGGCTACGTGGACGACACCCAGTTCGTGCGCTTCGACAAC
GACGCCGCGAGTCCGAGGATGGTGCCGCGGGCGCCGTGGATGGAGCAGGAGGGGT
CAGAGTATTGGGACCGGGAGACACGGAGCGCCAGGGACACCGCACAGATTTTCCGA
GTGAACCTGCGGACGCTGCGCGGCTACTACAATCAGAGCGAGGCCGGGTCTCACAC
CCTGCAGTGGATGCATGGCTGCGAGCTGGGGCCCGACGGGCGCTTCCTCCGCGGGT
ATGAACAGTTCGCCTACGACGGCAAGGATTATCTCACCCTGAATGAGGACCTGCGC
TCCTGGACCGCGGTGGACACGGCGGCTCAGATCTCCGAGCAAAAGTCAAATGATGC
CTCTGAGGCGGAGCACCAGAGAGCCTACCTGGAAGACACATGCGTGGAGTGGCTCC
ACAAATACCTGGAGAAGGGGAAGGAGACGCTGCTTCACCTGGAGCCCCCAAAGAC
ACACGTGACTCACCACCCCATCTCTGACCATGAGGCCACCCTGAGGTGCTGGGCTCT
GGGCTTCTACCCTGCGGAGATCACACTGACCTGGCAGCAGGATGGGGAGGGCCATA
CCCAGGACACGGAGCTCGTGGAGACCAGGCCTGCAGGGGATGGAACCTTCCAGAAG
TGGGCAGCTGTGGTGGTGCCTTCTGGAGAGGAGCAGAGATACACGTGCCATGTGCA
GCATGAGGGGCTACCCGAGCCCGTCACCCTGAGATGGAAGCCGGCTTCCCAGCCCA
CCATCCCCATCGTGGGCATCATTGCTGGCCTGGTTCTCCTTGGATCTGTGGTCTCTGG
AGCTGTGGTTGCTGCTGTGATATGGAGGAAGAAGAGCTCAGGTGGAAAAGGAGGG
AGCTACTCTAAGGCTGAGTGGAGCGACAGTGCCCAGGGGTCTGAGTCTCACAGCTT
G
SEQ ID NO: 12, HLA-E (HLA-E trimer minus signaling peptide)
GSHSLKYFHTSVSRPGRGEPRFISVGYVDDTQFVRFDNDAASPRMVPRAPWMEQEGSE
YWDRETRSARDTAQIFRVNLRTLRGYYNQSEAGSHTLQWMHGCELGPDGRFLRGYEQ
FAYDGKDYLTLNEDLRSWTAVDTAAQISEQKSNDASEAEHQRAYLEDTCVEWLHKYL
EKGKETLLHLEPPKTHVTHHPISDHEATLRCWALGFYPAEITLTWQQDGEGHTQDTELV
ETRPAGDGTFQKWAAVVVPSGEEQRYTCHVQHEGLPEPVTLRWKPASQPTIPIVGIIAGL
VLLGSVVSGAVVAAVIWRKKSSGGKGGSYSKAEWSDSAQGSESHSL
SEQ ID NO: 13, HLA-E construct sequence
ACTAGTGCCACCATGTCTCGCTCCGTGGCCTTAGCTGTGCTCGCGCTACTCTCTCTTT
CTGGCCTCGAGGCTGTTATGGCTCCGCGGACTTTAATTTTAGGTGGTGGCGGATCCG
GTGGTGGCGGTTCTGGTGGTGGCGGCTCCATCCAGCGTACGCCAAAGATTCAGGTTT
ACTCACGTCATCCAGCAGAGAATGGAAAGTCAAATTTCCTGAATTGCTATGTGTCTG
GGTTTCATCCATCCGACATTGAAGTTGACTTACTGAAGAATGGAGAGAGAATTGAA
AAAGTGGAGCATTCAGACTTGTCTTTCAGCAAGGACTGGTCTTTCTATCTCTTGTACT
ACACTGAGTTCACCCCCACTGAAAAAGATGAGTATGCCTGCCGTGTGAACCATGTG
ACTTTGTCACAGCCCAAGATAGTTAAGTGGGATCGCGACATGGGTGGTGGCGGTTCT
GGTGGTGGCGGTAGTGGCGGCGGAGGAAGCGGTGGTGGCGGTTCCGGATCTCACTC
CTTGAAGTATTTCCACACTTCCGTGTCCCGGCCCGGCCGCGGGGAGCCCCGCTTCAT
CTCTGTGGGCTACGTGGACGACACCCAGTTCGTGCGCTTCGACAACGACGCCGCGA
GTCCGAGGATGGTGCCGCGGGCGCCGTGGATGGAGCAGGAGGGGTCAGAGTATTGG
GACCGGGAGACACGGAGCGCCAGGGACACCGCACAGATTTTCCGAGTGAACCTGCG
GACGCTGCGCGGCTACTACAATCAGAGCGAGGCCGGGTCTCACACCCTGCAGTGGA
TGCATGGCTGCGAGCTGGGGCCCGACGGGCGCTTCCTCCGCGGGTATGAACAGTTC
GCCTACGACGGCAAGGATTATCTCACCCTGAATGAGGACCTGCGCTCCTGGACCGC
GGTGGACACGGCGGCTCAGATCTCCGAGCAAAAGTCAAATGATGCCTCTGAGGCGG
AGCACCAGAGAGCCTACCTGGAAGACACATGCGTGGAGTGGCTCCACAAATACCTG
GAGAAGGGGAAGGAGACGCTGCTTCACCTGGAGCCCCCAAAGACACACGTGACTCA
CCACCCCATCTCTGACCATGAGGCCACCCTGAGGTGCTGGGCTCTGGGCTTCTACCC
TGCGGAGATCACACTGACCTGGCAGCAGGATGGGGAGGGCCATACCCAGGACACG
GAGCTCGTGGAGACCAGGCCTGCAGGGGATGGAACCTTCCAGAAGTGGGCAGCTGT
GGTGGTGCCTTCTGGAGAGGAGCAGAGATACACGTGCCATGTGCAGCATGAGGGGC
TACCCGAGCCCGTCACCCTGAGATGGAAGCCGGCTTCCCAGCCCACCATCCCCATCG
TGGGCATCATTGCTGGCCTGGTTCTCCTTGGATCTGTGGTCTCTGGAGCTGTGGTTGC
TGCTGTGATATGGAGGAAGAAGAGCTCAGGTGGAAAAGGAGGGAGCTACTCTAAG
GCTGAGTGGAGCGACAGTGCCCAGGGGTCTGAGTCTCACAGCTTGTAAGAATTC
SEQ ID NO: 14, Nonamer for HLA-G
AAAGGACCTCCTGCCGCTCTTACACTT
SEQ ID NO: 15, Nonamer for HLA-G
KGPPAALTL
SEQ ID NO: 16, B2M signaling peptide (HLA-G trimer)
ATGTCTCGCTCCGTGGCCTTAGCTGTGCTCGCGCTACTCTCTCTTTCTGGCCTCGAGG
CT
SEQ ID NO: 17, B2M signaling peptide (HLA-G trimer)
MSRSVALAVLALLSLSGLEA
SEQ ID NO: 18, B2M minus signaling peptide (HLA-G trimer)
ATCCAGCGTACGCCAAAGATTCAGGTTTACTCACGTCATCCAGCAGAGAATGGAAA
GTCAAATTTCCTGAATTGCTATGTGTCTGGGTTTCATCCATCCGACATTGAAGTTGAC
TTACTGAAGAATGGAGAGAGAATTGAAAAAGTGGAGCATTCAGACTTGTCTTTCAG
CAAGGACTGGTCTTTCTATCTCTTGTACTACACTGAGTTCACCCCCACTGAAAAAGA
TGAGTATGCCTGCCGTGTGAACCATGTGACTTTGTCACAGCCCAAGATAGTTAAGTG
GGATCGCGACATG
SEQ ID NO: 19, B2M minus signaling peptide (HLA-G trimer)
IQRTPKIQVYSRHPAENGKSNFLNCYVSGFHPSDIEVDLLKNGERIEKVEHSDLSFSKDW
SFYLLYYTEFTPTEKDEYACRVNHVTLSQPKIVKWDRDM
SEQ ID NO: 20, HLA-G minus signaling peptide
GGCTCCCACTCCATGAGGTATTTCAGCGCCGCCGTGTCCCGGCCCGGCCGCGGGGA
GCCCCGCTTCATCGCCATGGGCTACGTGGACGACACGCAGTTCGTGCGGTTCGACAG
CGACTCGGCGTGTCCGAGGATGGAGCCGCGGGCGCCGTGGGTGGAGCAGGAGGGG
CCGGAGTATTGGGAAGAGGAGACACGGAACACCAAGGCCCACGCACAGACTGACA
GAATGAACCTGCAGACCCTGCGCGGCTACTACAACCAGAGCGAGGCCAGTTCTCAC
ACCCTCCAGTGGATGATTGGCTGCGACCTGGGGTCCGACGGACGCCTCCTCCGCGG
GTATGAACAGTATGCCTACGATGGCAAGGATTACCTCGCCCTGAACGAGGACCTGC
GCTCCTGGACCGCAGCGGACACTGCGGCTCAGATCTCCAAGCGCAAGTGTGAGGCG
GCCAATGTGGCTGAACAAAGGAGAGCCTACCTGGAGGGCACGTGCGTGGAGTGGCT
CCACAGATACCTGGAGAACGGGAAGGAGATGCTGCAGCGCGCGGACCCCCCCAAG
ACACACGTGACCCACCACCCTGTCTTTGACTATGAGGCCACCCTGAGGTGCTGGGCC
CTGGGCTTCTACCCTGCGGAGATCATACTGACCTGGCAGCGGGATGGGGAGGACCA
GACCCAGGACGTGGAGCTCGTGGAGACCAGGCCTGCAGGGGATGGAACCTTCCAGA
AGTGGGCAGCTGTGGTGGTGCCTTCTGGAGAGGAGCAGAGATACACGTGCCATGTG
CAGCATGAGGGGCTGCCGGAGCCCCTCATGCTGAGATGGAAGCAGTCTTCCCTGCC
CACCATCCCCATCATGGGTATCGTTGCTGGCCTGGTTGTCCTTGCAGCTGTAGTCACT
GGAGCTGCGGTCGCTGCTGTGCTGTGGAGAAAGAAGAGCTCAGATTGA
SEQ ID NO: 21, HLA-G minus signaling peptide
GSHSMRYFSAAVSRPGRGEPRFIAMGYVDDTQFVRFDSDSACPRMEPRAPWVEQEGPE
YWEEETRNTKAHAQTDRMNLQTLRGYYNQSEASSHTLQWMIGCDLGSDGRLLRGYEQ
YAYDGKDYLALNEDLRSWTAADTAAQISKRKCEAANVAEQRRAYLEGTCVEWLHRY
LENGKEMLQRADPPKTHVTHHPVFDYEATLRCWALGFYPAEIILTWQRDGEDQTQDVE
LVETRPAGDGTFQKWAAVVVPSGEEQRYTCHVQHEGLPEPLMLRWKQSSLPTIPIMGIV
AGLVVLAAVVTGAAVAAVLWRKKSSD
SEQ ID NO: 22, HLA-G trimer construct sequence
ACTAGTGCCACCATGTCTCGCTCCGTGGCCTTAGCTGTGCTCGCGCTACTCTCTCTTT
CTGGCCTCGAGGCTAAAGGACCTCCTGCCGCTCTTACACTTGGTGGTGGCGGATCCG
GTGGTGGCGGTTCTGGTGGTGGCGGCTCCATCCAGCGTACGCCAAAGATTCAGGTTT
ACTCACGTCATCCAGCAGAGAATGGAAAGTCAAATTTCCTGAATTGCTATGTGTCTG
GGTTTCATCCATCCGACATTGAAGTTGACTTACTGAAGAATGGAGAGAGAATTGAA
AAAGTGGAGCATTCAGACTTGTCTTTCAGCAAGGACTGGTCTTTCTATCTCTTGTACT
ACACTGAGTTCACCCCCACTGAAAAAGATGAGTATGCCTGCCGTGTGAACCATGTG
ACTTTGTCACAGCCCAAGATAGTTAAGTGGGATCGCGACATGGGTGGTGGCGGTTCT
GGTGGTGGCGGTAGTGGCGGCGGAGGAAGCGGTGGTGGCGGTTCCGGCTCCCACTC
CATGAGGTATTTCAGCGCCGCCGTGTCCCGGCCCGGCCGCGGGGAGCCCCGCTTCAT
CGCCATGGGCTACGTGGACGACACGCAGTTCGTGCGGTTCGACAGCGACTCGGCGT
GTCCGAGGATGGAGCCGCGGGCGCCGTGGGTGGAGCAGGAGGGGCCGGAGTATTG
GGAAGAGGAGACACGGAACACCAAGGCCCACGCACAGACTGACAGAATGAACCTG
CAGACCCTGCGCGGCTACTACAACCAGAGCGAGGCCAGTTCTCACACCCTCCAGTG
GATGATTGGCTGCGACCTGGGGTCCGACGGACGCCTCCTCCGCGGGTATGAACAGT
ATGCCTACGATGGCAAGGATTACCTCGCCCTGAACGAGGACCTGCGCTCCTGGACC
GCAGCGGACACTGCGGCTCAGATCTCCAAGCGCAAGTGTGAGGCGGCCAATGTGGC
TGAACAAAGGAGAGCCTACCTGGAGGGCACGTGCGTGGAGTGGCTCCACAGATACC
TGGAGAACGGGAAGGAGATGCTGCAGCGCGCGGACCCCCCCAAGACACACGTGAC
CCACCACCCTGTCTTTGACTATGAGGCCACCCTGAGGTGCTGGGCCCTGGGCTTCTA
CCCTGCGGAGATCATACTGACCTGGCAGCGGGATGGGGAGGACCAGACCCAGGACG
TGGAGCTCGTGGAGACCAGGCCTGCAGGGGATGGAACCTTCCAGAAGTGGGCAGCT
GTGGTGGTGCCTTCTGGAGAGGAGCAGAGATACACGTGCCATGTGCAGCATGAGGG
GCTGCCGGAGCCCCTCATGCTGAGATGGAAGCAGTCTTCCCTGCCCACCATCCCCAT
CATGGGTATCGTTGCTGGCCTGGTTGTCCTTGCAGCTGTAGTCACTGGAGCTGCGGT
CGCTGCTGTGCTGTGGAGAAAGAAGAGCTCAGATTGAGAATTC
SEQ ID NO: 23, HLA-E (HLA-E-B2M dimer)
ATGGTAGATGGAACCCTCCTTTTACTCCTCTCGGAGGCCCTGGCCCTTACCCAGACC
TGGGCGGGCTCCCACTCCTTGAAGTATTTCCACACTTCCGTGTCCCGGCCCGGCCGC
GGGGAGCCCCGCTTCATCTCTGTGGGCTACGTGGACGACACCCAGTTCGTGCGCTTC
GACAACGACGCCGCGAGTCCGAGGATGGTGCCGCGGGCGCCGTGGATGGAGCAGG
AGGGGTCAGAGTATTGGGACCGGGAGACACGGAGCGCCAGGGACACCGCACAGAT
TTTCCGAGTGAATCTGCGGACGCTGCGCGGCTACTACAATCAGAGCGAGGCCGGGT
CTCACACCCTGCAGTGGATGCATGGCTGCGAGCTGGGGCCCGACGGGCGCTTCCTCC
GCGGGTATGAACAGTTCGCCTACGACGGCAAGGATTATCTCACCCTGAATGAGGAC
CTGCGCTCCTGGACCGCGGTGGACACGGCGGCTCAGATCTCCGAGCAAAAGTCAAA
TGATGCCTCTGAGGCGGAGCACCAGAGAGCCTACCTGGAAGACACATGCGTGGAGT
GGCTCCACAAATACCTGGAGAAGGGGAAGGAGACGCTGCTTCACCTGGAGCCCCCA
AAGACACACGTGACTCACCACCCCATCTCTGACCATGAGGCCACCCTGAGGTGCTG
GGCCCTGGGCTTCTACCCTGCGGAGATCACACTGACCTGGCAGCAGGATGGGGAGG
GCCATACCCAGGACACGGAGCTCGTGGAGACCAGGCCTGCAGGGGATGGAACCTTC
CAGAAGTGGGCAGCTGTGGTGGTGCCTTCTGGAGAGGAGCAGAGATACACGTGCCA
TGTGCAGCATGAGGGGCTACCCGAGCCCGTCACCCTGAGATGGAAGCCGGCTTCCC
AGCCCACCATCCCCATCGTGGGCATCATTGCTGGCCTGGTTCTCCTTGGATCTGTGG
TCTCTGGAGCTGTGGTTGCTGCTGTGATATGGAGGAAGAAGAGCTCAGGTGGAAAA
GGAGGGAGCTACTCTAAGGCTGAGTGGAGCGACAGTGCCCAGGGGTCTGAGTCTCA
CAGCTTG
SEQ ID NO: 24, HLA-E (HLA-E-B2M dimer)
MVDGTLLLLLSEALALTQTWAGSHSLKYFHTSVSRPGRGEPRFISVGYVDDTQFVRFDN
DAASPRMVPRAPWMEQEGSEYWDRETRSARDTAQIFRVNLRTLRGYYNQSEAGSHTL
QWMHGCELGPDGRFLRGYEQFAYDGKDYLTLNEDLRSWTAVDTAAQISEQKSNDASE
AEHQRAYLEDTCVEWLHKYLEKGKETLLHLEPPKTHVTHHPISDHEATLRCWALGFYP
AEITLTWQQDGEGHTQDTELVETRPAGDGTFQKWAAVVVPSGEEQRYTCHVQHEGLP
EPVTLRWKPASQPTIPIVGIIAGLVLLGSVVSGAVVAAVIWRKKSSGGKGGSYSKAEWS
DSAQGSESHSL
SEQ ID NO: 25, B2M (HLA-E-B2M dimer)
TCTCGCTCCGTGGCCTTAGCTGTGCTCGCGCTACTCTCTCTTTCTGGCCTGGAGGCTA
TCCAGCGTACTCCAAAGATTCAGGTTTACTCACGTCATCCAGCAGAGAATGGAAAG
TCAAATTTCCTGAATTGCTATGTGTCTGGGTTTCATCCATCCGACATTGAAGTTGACT
TACTGAAGAATGGAGAGAGAATTGAAAAAGTGGAGCATTCAGACTTGTCTTTCAGC
AAGGACTGGTCTTTCTATCTCTTGTACTACACTGAATTCACCCCCACTGAAAAAGAT
GAGTATGCCTGCCGTGTGAACCATGTGACTTTGTCACAGCCCAAGATAGTTAAGTGG
GATCGAGACATG
SEQ ID NO: 26, B2M (HLA-E-B2M dimer)
SRSVALAVLALLSLSGLEAIQRTPKIQVYSRHPAENGKSNFLNCYVSGFHPSDIEVDLLK
NGERIEKVEHSDLSFSKDWSFYLLYYTEFTPTEKDEYACRVNHVTLSQPKIVKWDRDM
SEQ ID NO: 27, linker.
GGGGSSGGGGSSGGGGSS
SEQ ID NO: 28, HLA-E-B2M dimer construct
ATGGTAGATGGAACCCTCCTTTTACTCCTCTCGGAGGCCCTGGCCCTTACCCAGACC
TGGGCGGGCTCCCACTCCTTGAAGTATTTCCACACTTCCGTGTCCCGGCCCGGCCGC
GGGGAGCCCCGCTTCATCTCTGTGGGCTACGTGGACGACACCCAGTTCGTGCGCTTC
GACAACGACGCCGCGAGTCCGAGGATGGTGCCGCGGGCGCCGTGGATGGAGCAGG
AGGGGTCAGAGTATTGGGACCGGGAGACACGGAGCGCCAGGGACACCGCACAGAT
TTTCCGAGTGAATCTGCGGACGCTGCGCGGCTACTACAATCAGAGCGAGGCCGGGT
CTCACACCCTGCAGTGGATGCATGGCTGCGAGCTGGGGCCCGACGGGCGCTTCCTCC
GCGGGTATGAACAGTTCGCCTACGACGGCAAGGATTATCTCACCCTGAATGAGGAC
CTGCGCTCCTGGACCGCGGTGGACACGGCGGCTCAGATCTCCGAGCAAAAGTCAAA
TGATGCCTCTGAGGCGGAGCACCAGAGAGCCTACCTGGAAGACACATGCGTGGAGT
GGCTCCACAAATACCTGGAGAAGGGGAAGGAGACGCTGCTTCACCTGGAGCCCCCA
AAGACACACGTGACTCACCACCCCATCTCTGACCATGAGGCCACCCTGAGGTGCTG
GGCCCTGGGCTTCTACCCTGCGGAGATCACACTGACCTGGCAGCAGGATGGGGAGG
GCCATACCCAGGACACGGAGCTCGTGGAGACCAGGCCTGCAGGGGATGGAACCTTC
CAGAAGTGGGCAGCTGTGGTGGTGCCTTCTGGAGAGGAGCAGAGATACACGTGCCA
TGTGCAGCATGAGGGGCTACCCGAGCCCGTCACCCTGAGATGGAAGCCGGCTTCCC
AGCCCACCATCCCCATCGTGGGCATCATTGCTGGCCTGGTTCTCCTTGGATCTGTGG
TCTCTGGAGCTGTGGTTGCTGCTGTGATATGGAGGAAGAAGAGCTCAGGTGGAAAA
GGAGGGAGCTACTCTAAGGCTGAGTGGAGCGACAGTGCCCAGGGGTCTGAGTCTCA
CAGCTTGGGTGGTGGTGGTAGTAGTGGTGGTGGTGGTAGTAGTGGTGGTGGTGGTA
GTAGTTCTCGCTCCGTGGCCTTAGCTGTGCTCGCGCTACTCTCTCTTTCTGGCCTGGA
GGCTATCCAGCGTACTCCAAAGATTCAGGTTTACTCACGTCATCCAGCAGAGAATGG
AAAGTCAAATTTCCTGAATTGCTATGTGTCTGGGTTTCATCCATCCGACATTGAAGT
TGACTTACTGAAGAATGGAGAGAGAATTGAAAAAGTGGAGCATTCAGACTTGTCTT
TCAGCAAGGACTGGTCTTTCTATCTCTTGTACTACACTGAATTCACCCCCACTGAAA
AAGATGAGTATGCCTGCCGTGTGAACCATGTGACTTTGTCACAGCCCAAGATAGTTA
AGTGGGATCGAGACATGTAA
SEQ ID NO: 29, B2M (B2M-HLAG dimer)
ATGTCTCGCTCCGTGGCCTTAGCTGTGCTCGCGCTACTCTCTCTTTCTGGCCTGGAGG
CTATCCAGCGTACTCCAAAGATTCAGGTTTACTCACGTCATCCAGCAGAGAATGGAA
AGTCAAATTTCCTGAATTGCTATGTGTCTGGGTTTCATCCATCCGACATTGAAGTTG
ACTTACTGAAGAATGGAGAGAGAATTGAAAAAGTGGAGCATTCAGACTTGTCTTTC
AGCAAGGACTGGTCTTTCTATCTCTTGTACTACACTGAGTTCACCCCCACTGAAAAA
GATGAGTATGCCTGCCGTGTGAACCATGTGACTTTGTCACAGCCCAAGATAGTTAAG
TGGGATCGAGACATG
SEQ ID NO: 30, B2M (B2M-HLAG dimer)
MSRSVALAVLALLSLSGLEAIQRTPKIQVYSRHPAENGKSNFLNCYVSGFHPSDIEVDLL
KNGERIEKVEHSDLSFSKDWSFYLLYYTEFTPTEKDEYACRVNHVTLSQPKIVKWDRD
M
SEQ ID NO: 31, HLA-G (B2M-HLAG dimer)
AAGACGCCAAGGATGGTGGTCATGGCGCCCCGAACCCTCTTCCTGCTGCTCTCGGGG
GCCCTGACCCTGACCGAGACCTGGGCGGGCTCCCACTCCATGAGGTATTTCAGCGCC
GCCGTGTCCCGGCCCGGCCGCGGGGAGCCCCGCTTCATCGCCATGGGCTACGTGGA
CGACACGCAGTTCGTGCGGTTCGACAGCGACTCGGCGTGTCCGAGGATGGAGCCGC
GGGCGCCGTGGGTGGAGCAGGAGGGGCCGGAGTATTGGGAAGAGGAGACACGGAA
CACCAAGGCCCACGCACAGACTGACAGAATGAACCTGCAGACCCTGCGCGGCTACT
ACAACCAGAGCGAGGCCAGTTCTCACACCCTCCAGTGGATGATTGGCTGCGACCTG
GGGTCCGACGGACGCCTCCTCCGCGGGTATGAACAGTATGCCTACGATGGCAAGGA
TTACCTCGCCCTGAACGAGGACCTGCGCTCCTGGACCGCAGCGGACACTGCGGCTC
AGATCTCCAAGCGCAAGTGTGAGGCGGCCAATGTGGCTGAACAAAGGAGAGCCTAC
CTGGAGGGCACGTGCGTGGAGTGGCTCCACAGATACCTGGAGAACGGGAAGGAGA
TGCTGCAGCGCGCGGACCCCCCCAAGACACACGTGACCCACCACCCTGTCTTTGACT
ATGAGGCCACCCTGAGGTGCTGGGCCCTGGGCTTCTACCCTGCGGAGATCATACTGA
CCTGGCAGCGGGATGGGGAGGACCAGACCCAGGACGTGGAGCTCGTGGAGACCAG
GCCTGCAGGGGATGGAACCTTCCAGAAGTGGGCAGCTGTGGTGGTGCCTTCTGGAG
AGGAGCAGAGATACACGTGCCATGTGCAGCATGAGGGGCTGCCGGAGCCCCTCATG
CTGAGATGGAAGCAGTCTTCCCTGCCCACCATCCCCATCATGGGTATCGTTGCTGGC
CTGGTTGTCCTTGCAGCTGTAGTCACTGGAGCTGCGGTCGCTGCTGTGCTGTGGAGA
AAGAAGAGCTCAGATTGA
SEQ ID NO: 32, HLA-G (B2M-HLAG dimer)
KTPRMVVMAPRTLFLLLSGALTLTETWAGSHSMRYFSAAVSRPGRGEPRFIAMGYVDD
TQFVRFDSDSACPRMEPRAPWVEQEGPEYWEEETRNTKAHAQTDRMNLQTLRGYYNQ
SEASSHTLQWMIGCDLGSDGRLLRGYEQYAYDGKDYLALNEDLRSWTAADTAAQISK
RKCEAANVAEQRRAYLEGTCVEWLHRYLENGKEMLQRADPPKTHVTHHPVFDYEATL
RCWALGFYPAEIILTWQRDGEDQTQDVELVETRPAGDGTFQKWAAVVVPSGEEQRYTC
HVQHEGLPEPLMLRWKQSSLPTIPIMGIVAGLVVLAAVVTGAAVAAVLWRKKSSD
SEQ ID NO: 33, B2M-HLAG dimer construct
GCCACCATGTCTCGCTCCGTGGCCTTAGCTGTGCTCGCGCTACTCTCTCTTTCTGGCC
TGGAGGCTATCCAGCGTACTCCAAAGATTCAGGTTTACTCACGTCATCCAGCAGAGA
ATGGAAAGTCAAATTTCCTGAATTGCTATGTGTCTGGGTTTCATCCATCCGACATTG
AAGTTGACTTACTGAAGAATGGAGAGAGAATTGAAAAAGTGGAGCATTCAGACTTG
TCTTTCAGCAAGGACTGGTCTTTCTATCTCTTGTACTACACTGAGTTCACCCCCACTG
AAAAAGATGAGTATGCCTGCCGTGTGAACCATGTGACTTTGTCACAGCCCAAGATA
GTTAAGTGGGATCGAGACATGGGTGGTGGTGGTAGTGGTGGTGGTGGTAGTGGTGG
TGGTGGTAGTAAGACGCCAAGGATGGTGGTCATGGCGCCCCGAACCCTCTTCCTGCT
GCTCTCGGGGGCCCTGACCCTGACCGAGACCTGGGCGGGCTCCCACTCCATGAGGT
ATTTCAGCGCCGCCGTGTCCCGGCCCGGCCGCGGGGAGCCCCGCTTCATCGCCATGG
GCTACGTGGACGACACGCAGTTCGTGCGGTTCGACAGCGACTCGGCGTGTCCGAGG
ATGGAGCCGCGGGCGCCGTGGGTGGAGCAGGAGGGGCCGGAGTATTGGGAAGAGG
AGACACGGAACACCAAGGCCCACGCACAGACTGACAGAATGAACCTGCAGACCCT
GCGCGGCTACTACAACCAGAGCGAGGCCAGTTCTCACACCCTCCAGTGGATGATTG
GCTGCGACCTGGGGTCCGACGGACGCCTCCTCCGCGGGTATGAACAGTATGCCTAC
GATGGCAAGGATTACCTCGCCCTGAACGAGGACCTGCGCTCCTGGACCGCAGCGGA
CACTGCGGCTCAGATCTCCAAGCGCAAGTGTGAGGCGGCCAATGTGGCTGAACAAA
GGAGAGCCTACCTGGAGGGCACGTGCGTGGAGTGGCTCCACAGATACCTGGAGAAC
GGGAAGGAGATGCTGCAGCGCGCGGACCCCCCCAAGACACACGTGACCCACCACCC
TGTCTTTGACTATGAGGCCACCCTGAGGTGCTGGGCCCTGGGCTTCTACCCTGCGGA
GATCATACTGACCTGGCAGCGGGATGGGGAGGACCAGACCCAGGACGTGGAGCTCG
TGGAGACCAGGCCTGCAGGGGATGGAACCTTCCAGAAGTGGGCAGCTGTGGTGGTG
CCTTCTGGAGAGGAGCAGAGATACACGTGCCATGTGCAGCATGAGGGGCTGCCGGA
GCCCCTCATGCTGAGATGGAAGCAGTCTTCCCTGCCCACCATCCCCATCATGGGTAT
CGTTGCTGGCCTGGTTGTCCTTGCAGCTGTAGTCACTGGAGCTGCGGTCGCTGCTGT
GCTGTGGAGAAAGAAGAGCTCAGATTGA
SEQ ID NO: 34, AAVS1-HLA-E trimer construct
AGTCCTGCAGGTTTAAACGAATTCGCCCTTTGCTTTCTCTGACCAGCATTCTCTCCCC
TGGGCCTGTGCCGCTTTCTGTCTGCAGCTTGTGGCCTGGGTCACCTCTACGGCTGGC
CCAGATCCTTCCCTGCCGCCTCCTTCAGGTTCCGTCTTCCTCCACTCCCTCTTCCCCTT
GCTCTCTGCTGTGTTGCTGCCCAAGGATGCTCTTTCCGGAGCACTTCCTTCTCGGCGC
TGCACCACGTGATGTCCTCTGAGCGGATCCTCCCCGTGTCTGGGTCCTCTCCGGGCA
TCTCTCCTCCCTCACCCAACCCCATGCCGTCTTCACTCGCTGGGTTCCCTTTTCCTTCT
CCTTCTGGGGCCTGTGCCATCTCTCGTTTCTTAGGATGGCCTTCTCCGACGGATGTCT
CCCTTGCGTCCCGCCTCCCCTTCTTGTAGGCCTGCATCATCACCGTTTTTCTGGACAA
CCCCAAAGTACCCCGTCTCCCTGGCTTTAGCCACCTCTCCATCCTCTTGCTTTCTTTG
CCTGGACACCCCGTTCTCCTGTGGATTCGGGTCACCTCTCACTCCTTTCATTTGGGCA
GCTCCCCTACCCCCCTTACCTCTCTAGTCTGTGCTAGCTCTTCCAGCCCCCTGTCATG
GCATCTTCCAGGGGTCCGAGAGCTCAGCTAGTCTTCTTCCTCCAACCCGGGCCCCTA
TGTCCACTTCAGGACAGCATGTTTGCTGCCTCCAGGGATCCTGTGTCCCCGAGCTGG
GACCACCTTATATTCCCAGGGCCGGTTAATGTGGCTCTGGTTCTGGGTACTTTTATCT
GTCCCCTCCACCCCACAGTGGGGCAAGCTTCTGACCTCTTCTCTTCCTCCCACAGGG
CCTCGAGAGATCTGGCAGCGGAGAGGGCAGAGGAAGTCTTCTAACATGCGGTGACG
TGGAGGAGAATCCCGGCCCTAGGCTCGAGATGACCGAGTACAAGCCCACGGTGCGC
CTCGCCACCCGCGACGACGTCCCCAGGGCCGTACGCACCCTCGCCGCCGCGTTCGCC
GACTACCCCGCCACGCGCCACACCGTCGATCCGGACCGCCACATCGAGCGGGTCAC
CGAGCTGCAAGAACTCTTCCTCACGCGCGTCGGGCTCGACATCGGCAAGGTGTGGG
TCGCGGACGACGGCGCCGCGGTGGCGGTCTGGACCACGCCGGAGAGCGTCGAAGCG
GGGGCGGTGTTCGCCGAGATCGGCCCGCGCATGGCCGAGTTGAGCGGTTCCCGGCT
GGCCGCGCAGCAACAGATGGAAGGCCTCCTGGCGCCGCACCGGCCCAAGGAGCCCG
CGTGGTTCCTGGCCACCGTCGGCGTCTCGCCCGACCACCAGGGCAAGGGTCTGGGC
AGCGCCGTCGTGCTCCCCGGAGTGGAGGCGGCCGAGCGCGCCGGGGTGCCCGCCTT
CCTGGAGACCTCCGCGCCCCGCAACCTCCCCTTCTACGAGCGGCTCGGCTTCACCGT
CACCGCCGACGTCGAGGTGCCCGAAGGACCGCGCACCTGGTGCATGACCCGCAAGC
CCGGTGCCTGATCTAGAGGGCCCGTTTAAACCCGCTGATCAGCCTCGACTGTGCCTT
CTAGTTGCCAGCCATCTGTTGTTTGCCCCTCCCCCGTGCCTTCCTTGACCCTGGAAGG
TGCCACTCCCACTGTCCTTTCCTAATAAAATGAGGAAATTGCATCGCATTGTCTGAG
TAGGTGTCATTCTATTCTGGGGGGTGGGGTGGGGCAGGACAGCAAGGGGGAGGATT
GGGAAGACAATAGCAGGCATGCTGGGGATGCGGTGGGCTCTATGGGTCTCGACATT
GATTATTGACTAGTTATTAATAGTAATCAATTACGGGGTCATTAGTTCATAGCCCAT
ATATGGAGTTCCGCGTTACATAACTTACGGTAAATGGCCCGCCTGGCTGACCGCCCA
ACGACCCCCGCCCATTGACGTCAATAATGACGTATGTTCCCATAGTAACGCCAATAG
GGACTTTCCATTGACGTCAATGGGTGGAGTATTTACGGTAAACTGCCCACTTGGCAG
TACATCAAGTGTATCATATGCCAAGTACGCCCCCTATTGACGTCAATGACGGTAAAT
GGCCCGCCTGGCATTATGCCCAGTACATGACCTTATGGGACTTTCCTACTTGGCAGT
ACATCTACGTATTAGTCATCGCTATTACCATGGTCGAGGTGAGCCCCACGTTCTGCT
TCACTCTCCCCATCTCCCCCCCCTCCCCACCCCCAATTTTGTATTTATTTATTTTTTAA
TTATTTTGTGCAGCGATGGGGGCGGGGGGGGGGGGGGGCGAGGCGGAGAGGTGCG
GCGGCAGCCAATCAGAGCGGCGCGCTCCGAAAGTTTCCTTTTATGGCGAGGCGGCG
GCGGCGGCGGCCCTATAAAAAGCGAAGCGCGCGGCGGGCGGGAGTCGCTGCGCGC
TGCCTTCGCCCCGTGCCCCGCTCCGCCGCCGCCTCGCGCCGCCCGCCCCGGCTCTGA
CTGACCGCGTTACTCCCACAGGTGAGCGGGCGGGACGGCCCTTCTCCTCCGGGCTGT
AATTAGCGCTTGGTTTAATGACGGCTTGTTTCTTTTCTGTGGCTGCGTGAAAGCCTTG
AGGGGCTCCGGGAGGGCCCTTTGTGCGGGGGGAGCGGCTCGGGGGGTGCGTGCGTG
TGTGTGTGCGTGGGGAGCGCCGCGTGCGGCTCCGCGCTGCCCGGCGGCTGTGAGCG
CTGCGGGCGCGGCGCGGGGCTTTGTGCGCTCCGCAGTGTGCGCGAGGGGAGCGCGG
CCGGGGGCGGTGCCCCGCGGTGCGGGGGGGGCTGCGAGGGGAACAAAGGCTGCGT
GCGGGGTGTGTGCGTGGGGGGGTGAGCAGGGGGTGTGGGCGCGTCGGTCGGGCTGC
AACCCCCCCTGCACCCCCCTCCCCGAGTTGCTGAGCACGGCCCGGCTTCGGGTGCGG
GGCTCCGTACGGGGCGTGGCGCGGGGCTCGCCGTGCCGGGCGGGGGGTGGCGGCAG
GTGGGGGTGCCGGGCGGGGGGGGGCCGCCTCGGGCCGGGGAGGGCTCGGGGGAGG
GGCGCGGCGGCCCCCGGAGCGCCGGCGGCTGTCGAGGCGCGGCGAGCCGCAGCCAT
TGCCTTTTATGGTAATCGTGCGAGAGGGCGCAGGGACTTCCTTTGTCCCAAATCTGT
GCGGAGCCGAAATCTGGGAGGCGCCGCCGCACCCCCTCTAGCGGGCGCGGGGCGAA
GCGGTGCGGCGCCGGCAGGAAGGAAATGGGCGGGGAGGGCCTTCGTGCGTCGCCG
CGCCGCCGTCCCCTTCTCCCTCTCCAGCCTCGGGGCTGTCCGCGGGGGGACGGCTGC
CTTCGGGGGGGACGGGGCAGGGCGGGGTTCGGCTTCTGGCGTGTGACCGGCGGCTC
TAGAGCCTCTGCTAACCATGTTCATGCCTTCTTCTTTTTCCTACAGCTCCTGGGCAAC
GTGCTGGTTATTGTGCTGTCTCATCATTTTGGCAAAGAATTCGTCGACATGTCTCGCT
CCGTGGCCTTAGCTGTGCTCGCGCTACTCTCTCTTTCTGGCCTCGAGGCTGTTATGGC
TCCGCGGACTTTAATTTTAGGTGGTGGCGGATCCGGTGGTGGCGGTTCTGGTGGTGG
CGGCTCCATCCAGCGTACGCCAAAGATTCAGGTTTACTCACGTCATCCAGCAGAGA
ATGGAAAGTCAAATTTCCTGAATTGCTATGTGTCTGGGTTTCATCCATCCGACATTG
AAGTTGACTTACTGAAGAATGGAGAGAGAATTGAAAAAGTGGAGCATTCAGACTTG
TCTTTCAGCAAGGACTGGTCTTTCTATCTCTTGTACTACACTGAGTTCACCCCCACTG
AAAAAGATGAGTATGCCTGCCGTGTGAACCATGTGACTTTGTCACAGCCCAAGATA
GTTAAGTGGGATCGCGACATGGGTGGTGGCGGTTCTGGTGGTGGCGGTAGTGGCGG
CGGAGGAAGCGGTGGTGGCGGTTCCGGATCTCACTCCTTGAAGTATTTCCACACTTC
CGTGTCCCGGCCCGGCCGCGGGGAGCCCCGCTTCATCTCTGTGGGCTACGTGGACGA
CACCCAGTTCGTGCGCTTCGACAACGACGCCGCGAGTCCGAGGATGGTGCCGCGGG
CGCCGTGGATGGAGCAGGAGGGGTCAGAGTATTGGGACCGGGAGACACGGAGCGC
CAGGGACACCGCACAGATTTTCCGAGTGAACCTGCGGACGCTGCGCGGCTACTACA
ATCAGAGCGAGGCCGGGTCTCACACCCTGCAGTGGATGCATGGCTGCGAGCTGGGG
CCCGACGGGCGCTTCCTCCGCGGGTATGAACAGTTCGCCTACGACGGCAAGGATTA
TCTCACCCTGAATGAGGACCTGCGCTCCTGGACCGCGGTGGACACGGCGGCTCAGA
TCTCCGAGCAAAAGTCAAATGATGCCTCTGAGGCGGAGCACCAGAGAGCCTACCTG
GAAGACACATGCGTGGAGTGGCTCCACAAATACCTGGAGAAGGGGAAGGAGACGC
TGCTTCACCTGGAGCCCCCAAAGACACACGTGACTCACCACCCCATCTCTGACCATG
AGGCCACCCTGAGGTGCTGGGCTCTGGGCTTCTACCCTGCGGAGATCACACTGACCT
GGCAGCAGGATGGGGAGGGCCATACCCAGGACACGGAGCTCGTGGAGACCAGGCC
TGCAGGGGATGGAACCTTCCAGAAGTGGGCAGCTGTGGTGGTGCCTTCTGGAGAGG
AGCAGAGATACACGTGCCATGTGCAGCATGAGGGGCTACCCGAGCCCGTCACCCTG
AGATGGAAGCCGGCTTCCCAGCCCACCATCCCCATCGTGGGCATCATTGCTGGCCTG
GTTCTCCTTGGATCTGTGGTCTCTGGAGCTGTGGTTGCTGCTGTGATATGGAGGAAG
AAGAGCTCAGGTGGAAAAGGAGGGAGCTACTCTAAGGCTGAGTGGAGCGACAGTG
CCCAGGGGTCTGAGTCTCACAGCTTGTAAACGCGTGAATTCGATATCAAGCTTATCG
ATAATCAACCTCTGGATTACAAAATTTGTGAAAGATTGACTGGTATTCTTAACTATG
TTGCTCCTTTTACGCTATGTGGATACGCTGCTTTAATGCCTTTGTATCATGCTATTGC
TTCCCGTATGGCTTTCATTTTCTCCTCCTTGTATAAATCCTGGTTGCTGTCTCTTTATG
AGGAGTTGTGGCCCGTTGTCAGGCAACGTGGCGTGGTGTGCACTGTGTTTGCTGACG
CAACCCCCACTGGTTGGGGCATTGCCACCACCTGTCAGCTCCTTTCCGGGACTTTCG
CTTTCCCCCTCCCTATTGCCACGGCGGAACTCATCGCCGCCTGCCTTGCCCGCTGCTG
GACAGGGGCTCGGCTGTTGGGCACTGACAATTCCGTGGTGTTGTCGGGGAAATCAT
CGTCCTTTCCTTGGCTGCTCGCCTATGTTGCCACCTGGATTCTGCGCGGGACGTCCTT
CTGCTACGTCCCTTCGGCCCTCAATCCAGCGGACCTTCCTTCCCGCGGCCTGCTGCC
GGCTCTGCGGCCTCTTCCGCGTCTTCGCCTTCGCCCTCAGACGAGTCGGATCTCCCTT
TGGGCCGCCTCCCCGCATCGATACCGAGCGCTGCTCGAGAGATCTACGGGTGGCAT
CCCTGTGACCCCTCCCCAGTGCCTCTCCTGGCCCTGGAAGTTGCCACTCCAGTGCCC
ACCAGCCTTGTCCTAATAAAATTAAGTTGCATCATTTTGTCTGACTAGGTGTCCTTCT
ATAATATTATGGGGTGGAGGGGGGTGGTATGGAGCAAGGGGCAAGTTGGGAAGAC
AACCTGTAGGGCCTGCGGGGTCTATTGGGAACCAAGCTGGAGTGCAGTGGCACAAT
CTTGGCTCACTGCAATCTCCGCCTCCTGGGTTCAAGCGATTCTCCTGCCTCAGCCTCC
CGAGTTGTTGGGATTCCAGGCATGCATGACCAGGCTCAGCTAATTTTTGTTTTTTTGG
TAGAGACGGGGTTTCACCATATTGGCCAGGCTGGTCTCCAACTCCTAATCTCAGGTG
ATCTACCCACCTTGGCCTCCCAAATTGCTGGGATTACAGGCGTGAACCACTGCTCCC
TTCCCTGTCCTTCTGATTTTGTAGGTAACCAGGCGCGTGAATTCACTCCTCAGGTGCA
GGCTGCCTATCAGAAGGTGGTGGCTGGTGTGGCCAATGCCCTGGCTCACAAATACC
ACTGAGATCTTTTTCCCTCTGCCAAAAATTATGGGGACATCATGAAGCCCCTTGAGC
ATCTGACTTCTGGCTAATAAAGGAAATTTATTTTCATTGCAATAGTGTGTTGGAATTT
TTTGTGTCTCTCACTCGGAAGGACATATGGGAGGGCAAATCATTTAAAACATCAGA
ATGAGTATTTGGTTTAGAGTTTGGCAACATATGCCCATATGCTGGCTGCCATGAACA
AAGGTTGGCTATAAAGAGGTCATCAGTATATGAAACAGCCCCCTGCTGTCCATTCCT
TATTCCATAGAAAAGCCTTGACTTGAGGTTAGATTTTTTTTATATTTTGTTTTGTGTT
ATTTTTTTCTTTAACATCCCTAAAATTTTCCTTACATGTTTTACTAGCCAGATTTTTCC
TCCTCTCCTGACTACTCCCAGTCATAGCTGTCCCTCTTCTCTTATGGAGATCCCTCGA
CCTGCAGCCCAAGCTTGGCGTAATCATGGTCATAGCTGTTTCCTGTGTGAAATTGTT
ATCCGCTCACAATTCCACACAACATACGAGCCGGAAGCATAAAGTGTAAAGCCTGG
GGTGCCTAATGAGTGAGCTAACTCACATTAATTGCGTTGCGCTCACTGCCCGCTTTC
CAGTCGGGAAACCTGTCGTGCCAGCGGATCGACAGTACTAAGCTTTACTAGGGACA
GGATTGGTGACAGAAAAGCCCCATCCTTAGGCCTCCTCCTTCCTAGTCTCCTGATAT
TGGGTCTAACCCCCACCTCCTGTTAGGCAGATTCCTTATCTGGTGACACACCCCCAT
TTCCTGGAGCCATCTCTCTCCTTGCCAGAACCTCTAAGGTTTGCTTACGATGGAGCC
AGAGAGGATCCTGGGAGGGAGAGCTTGGCAGGGGGTGGGAGGGAAGGGGGGGATG
CGTGACCTGCCCGGTTCTCAGTGGCCACCCTGCGCTACCCTCTCCCAGAACCTGAGC
TGCTCTGACGCGGCTGTCTGGTGCGTTTCACTGATCCTGGTGCTGCAGCTTCCTTACA
CTTCCCAAGAGGAGAAGCAGTTTGGAAAAACAAAATCAGAATAAGTTGGTCCTGAG
TTCTAACTTTGGCTCTTCACCTTTCTAGTCCCCAATTTATATTGTTCCTCCGTGCGTCA
GTTTTACCTGTGAGATAAGGCCAGTAGCCAGCCCCGTCCTGGCAGGGCTGTGGTGA
GGAGGGGGGTGTCCGTGTGGAAAACTCCCTTTGTGAGAATGGTGCGTCCTAGGTGTT
CACCAGGTCGTGGCCGCCTCTACTCCCTTTCTCTTTCTCCATCCTTCTTTCCTTAAAG
AGTCCCCAGTGCTATCTGGGACATATTCCTCCGCCCAGAGCAGGGTCCCGCTTCCCT
AAGGCCCTGCTCTGGGCTTCTGGGTTTGAGTCCTTGGCAAGCCCAGGAGAGGCGCTC
AGGCTTCCCTGTCCCCCTTCCTCGTCCACCATCTCATGCCCCTGGCTCTCCTGCCCCT
TCCCTACAGGGGTTCCTGGCTCTGCTCTAAGGGCGAATTCGCGGCCGCTAAATTCAA
TTCGCCCTATAGTGAGTCGTATTACAATTCACTGGCCGTCGTTTTACAACGTCGTGA
CTGGGAAAACCCTGGCGTTACCCAACTTAATCGCCTTGCAGCACATCCCCCTTTCGC
CAGCTGGCGTAATAGCGAAGAGGCCCGCACCGATCGCCCTTCCCAACAGTTGCGCA
GCCTATACGTACGGCAGTTTAAGGTTTACACCTATAAAAGAGAGAGCCGTTATCGTC
TGTTTGTGGATGTACAGAGTGATATTATTGACACGCCGGGGCGACGGATGGTGATCC
CCCTGGCCAGTGCACGTCTGCTGTCAGATAAAGTCTCCCGTGAACTTTACCCGGTGG
TGCATATCGGGGATGAAAGCTGGCGCATGATGACCACCGATATGGCCAGTGTGCCG
GTCTCCGTTATCGGGGAAGAAGTGGCTGATCTCAGCCACCGCGAAAATGACATCAA
AAACGCCATTAACCTGATGTTCTGGGGAATATAAATGTCAGGCATGAGATTATCAA
AAAGGATCTTCACCTAGATCCTTTTCACGTAGAAAGCCAGTCCGCAGAAACGGTGCT
GACCCCGGATGAATGTCAGCTACTGGGCTATCTGGACAAGGGAAAACGCAAGCGCA
AAGAGAAAGCAGGTAGCTTGCAGTGGGCTTACATGGCGATAGCTAGACTGGGCGGT
TTTATGGACAGCAAGCGAACCGGAATTGCCAGCTGGGGCGCCCTCTGGTAAGGTTG
GGAAGCCCTGCAAAGTAAACTGGATGGCTTTCTTGCCGCCAAGGATCTGATGGCGC
AGGGGATCAAGCTCTGATCAAGAGACAGGATGAGGATCGTTTCGCATGATTGAACA
AGATGGATTGCACGCAGGTTCTCCGGCCGCTTGGGTGGAGAGGCTATTCGGCTATG
ACTGGGCACAACAGACAATCGGCTGCTCTGATGCCGCCGTGTTCCGGCTGTCAGCGC
AGGGGCGCCCGGTTCTTTTTGTCAAGACCGACCTGTCCGGTGCCCTGAATGAACTGC
AAGACGAGGCAGCGCGGCTATCGTGGCTGGCCACGACGGGCGTTCCTTGCGCAGCT
GTGCTCGACGTTGTCACTGAAGCGGGAAGGGACTGGCTGCTATTGGGCGAAGTGCC
GGGGCAGGATCTCCTGTCATCTCACCTTGCTCCTGCCGAGAAAGTATCCATCATGGC
TGATGCAATGCGGCGGCTGCATACGCTTGATCCGGCTACCTGCCCATTCGACCACCA
AGCGAAACATCGCATCGAGCGAGCACGTACTCGGATGGAAGCCGGTCTTGTCGATC
AGGATGATCTGGACGAAGAGCATCAGGGGCTCGCGCCAGCCGAACTGTTCGCCAGG
CTCAAGGCGAGCATGCCCGACGGCGAGGATCTCGTCGTGACCCATGCATGGCGATG
CCTGCTTGCCGAATATCATGGTGGAAAATGGCCGCTTTTCTGGATTCATCGACTGTG
GCCGGCTGGGTGTGGCGGACCGCTATCAGGACATAGCGTTGGCTACCCGTGATATT
GCTGAAGAGCTTGGCGGCGAATGGGCTGACCGCTTCCTCGTGCTTTACGGTATCGCC
GCTCCCGATTCGCAGCGCATCGCCTTCTATCGCCTTCTTGACGAGTTCTTCTGAATTA
TTAACGCTTACAATTTCCTGATGCGGTATTTTCTCCTTACGCATCTGTGCGGTATTTC
ACACCGCATCAGGTGGCACTTTTCGGGGAAATGTGCGCGGAACCCCTATTTGTTTAT
TTTTCTAAATACATTCAAATATGTATCCGCTCATGAGATTATCAAAAAGGATCTTCA
CCTAGATCCTTTTAAATTAAAAATGAAGTTTTAAATCAATCTAAAGTATATATGAGT
AAACTTGGTCTGACAGTTACCAATGCTTAATCAGTGAGGCACCTATCTCAGCGATCT
GTCTATTTCGTTCATCCATAGTTGCCTGACTCCCCGTCGTGTAGATAACTACGATACG
GGAGGGCTTACCATCTGGCCCCAGTGCTGCAATGATACCGCGAGACCCACGCTCAC
CGGCTCCAGATTTATCAGCAATAAACCAGCCAGCCGGAAGGGCCGAGCGCAGAAGT
GGTCCTGCAACTTTATCCGCCTCCATCCAGTCTATTAATTGTTGCCGGGAAGCTAGA
GTAAGTAGTTCGCCAGTTAATAGTTTGCGCAACGTTGTTGCCATTGCTACAGGCATC
GTGGTGTCACGCTCGTCGTTTGGTATGGCTTCATTCAGCTCCGGTTCCCAACGATCA
AGGCGAGTTACATGATCCCCCATGTTGTGCAAAAAAGCGGTTAGCTCCTTCGGTCCT
CCGATCGTTGTCAGAAGTAAGTTGGCCGCAGTGTTATCACTCATGGTTATGGCAGCA
CTGCATAATTCTCTTACTGTCATGCCATCCGTAAGATGCTTTTCTGTGACTGGTGAAT
ACTCAACCAAGTCATTCTGAGAATAGTGTATGCGGCGACCGAGTTGCTCTTGCCCGG
CGTCAATACGGGATAATACCGCGCCACATAGCAGAACTTTAAAAGTGCTCATCATT
GGAAAACGTTCTTCGGGGCGAAAACTCTCAAGGATCTTACCGCTGTTGAGATCCAGT
TCGATGTAACCCACTCGTGCACCCAACTGATCTTCAGCATCTTTTACTTTCACCAGCG
TTTCTGGGTGAGCAAAAACAGGAAGGCAAAATGCCGCAAAAAAGGGAATAAGGGC
GACACGGAAATGTTGAATACTCATACTCTTCCTTTTTCAATATTATTGAAGCATTTAT
CAGGGTTATTGTCTCATGACCAAAATCCCTTAACGTGAGTTTTCGTTCCACTGAGCG
TCAGACCCCGTAGAAAAGATCAAAGGATCTTCTTGAGATCCTTTTTTTCTGCGCGTA
ATCTGCTGCTTGCAAACAAAAAAACCACCGCTACCAGCGGTGGTTTGTTTGCCGGAT
CAAGAGCTACCAACTCTTTTTCCGAAGGTAACTGGCTTCAGCAGAGCGCAGATACC
AAATACTGTTCTTCTAGTGTAGCCGTAGTTAGGCCACCACTTCAAGAACTCTGTAGC
ACCGCCTACATACCTCGCTCTGCTAATCCTGTTACCAGTGGCTGCTGCCAGTGGCGA
TAAGTCGTGTCTTACCGGGTTGGACTCAAGACGATAGTTACCGGATAAGGCGCAGC
GGTCGGGCTGAACGGGGGGTTCGTGCACACAGCCCAGCTTGGAGCGAACGACCTAC
ACCGAACTGAGATACCTACAGCGTGAGCTATGAGAAAGCGCCACGCTTCCCGAAGG
GAGAAAGGCGGACAGGTATCCGGTAAGCGGCAGGGTCGGAACAGGAGAGCGCACG
AGGGAGCTTCCAGGGGGAAACGCCTGGTATCTTTATAGTCCTGTCGGGTTTCGCCAC
CTCTGACTTGAGCGTCGATTTTTGTGATGCTCGTCAGGGGGGCGGAGCCTATGGAAA
AACGCCAGCAACGCGGCCTTTTTACGGTTCCTGGCCTTTTGCTGGCCTTTTGCTCACA
TGTTCTTTCCTGCGTTATCCCCTGATTCTGTGGATAACCGTATTACCGCCTTTGAGTG
AGCTGATACCGCTCGCCGCAGCCGAACGACCGAGCGCAGCGAGTCAGTGAGCGAGG
AAGCGGAAGAGCGCCCAATACGCAAACCGCCTCTCCCCGCGCGTTGGCCGATTCAT
TAATGCAGCTGGCACGACAGGTTTCCCGACTGGAAAGCGGGCAGTGAGCGCAACGC
AATTAATGTGAGTTAGCTCACTCATTAGGCACCCCAGGCTTTACACTTTATGCTTCC
GGCTCGTATGTTGTGTGGAATTGTGAGCGGATAACAATTTCACACAGGAAACAGCT
ATGACCATGATTACGCCAAGCTCAGAATTAACCCTCACTAAAGGGACTAGCT
SEQ ID NO: 35, AAVS1-HLA-G trimer construct
AGTCCTGCAGGTTTAAACGAATTCGCCCTTTGCTTTCTCTGACCAGCATTCTCTCCCC
TGGGCCTGTGCCGCTTTCTGTCTGCAGCTTGTGGCCTGGGTCACCTCTACGGCTGGC
CCAGATCCTTCCCTGCCGCCTCCTTCAGGTTCCGTCTTCCTCCACTCCCTCTTCCCCTT
GCTCTCTGCTGTGTTGCTGCCCAAGGATGCTCTTTCCGGAGCACTTCCTTCTCGGCGC
TGCACCACGTGATGTCCTCTGAGCGGATCCTCCCCGTGTCTGGGTCCTCTCCGGGCA
TCTCTCCTCCCTCACCCAACCCCATGCCGTCTTCACTCGCTGGGTTCCCTTTTCCTTCT
CCTTCTGGGGCCTGTGCCATCTCTCGTTTCTTAGGATGGCCTTCTCCGACGGATGTCT
CCCTTGCGTCCCGCCTCCCCTTCTTGTAGGCCTGCATCATCACCGTTTTTCTGGACAA
CCCCAAAGTACCCCGTCTCCCTGGCTTTAGCCACCTCTCCATCCTCTTGCTTTCTTTG
CCTGGACACCCCGTTCTCCTGTGGATTCGGGTCACCTCTCACTCCTTTCATTTGGGCA
GCTCCCCTACCCCCCTTACCTCTCTAGTCTGTGCTAGCTCTTCCAGCCCCCTGTCATG
GCATCTTCCAGGGGTCCGAGAGCTCAGCTAGTCTTCTTCCTCCAACCCGGGCCCCTA
TGTCCACTTCAGGACAGCATGTTTGCTGCCTCCAGGGATCCTGTGTCCCCGAGCTGG
GACCACCTTATATTCCCAGGGCCGGTTAATGTGGCTCTGGTTCTGGGTACTTTTATCT
GTCCCCTCCACCCCACAGTGGGGCAAGCTTCTGACCTCTTCTCTTCCTCCCACAGGG
CCTCGAGAGATCTGGCAGCGGAGAGGGCAGAGGAAGTCTTCTAACATGCGGTGACG
TGGAGGAGAATCCCGGCCCTAGGCTCGAGATGACCGAGTACAAGCCCACGGTGCGC
CTCGCCACCCGCGACGACGTCCCCAGGGCCGTACGCACCCTCGCCGCCGCGTTCGCC
GACTACCCCGCCACGCGCCACACCGTCGATCCGGACCGCCACATCGAGCGGGTCAC
CGAGCTGCAAGAACTCTTCCTCACGCGCGTCGGGCTCGACATCGGCAAGGTGTGGG
TCGCGGACGACGGCGCCGCGGTGGCGGTCTGGACCACGCCGGAGAGCGTCGAAGCG
GGGGCGGTGTTCGCCGAGATCGGCCCGCGCATGGCCGAGTTGAGCGGTTCCCGGCT
GGCCGCGCAGCAACAGATGGAAGGCCTCCTGGCGCCGCACCGGCCCAAGGAGCCCG
CGTGGTTCCTGGCCACCGTCGGCGTCTCGCCCGACCACCAGGGCAAGGGTCTGGGC
AGCGCCGTCGTGCTCCCCGGAGTGGAGGCGGCCGAGCGCGCCGGGGTGCCCGCCTT
CCTGGAGACCTCCGCGCCCCGCAACCTCCCCTTCTACGAGCGGCTCGGCTTCACCGT
CACCGCCGACGTCGAGGTGCCCGAAGGACCGCGCACCTGGTGCATGACCCGCAAGC
CCGGTGCCTGATCTAGAGGGCCCGTTTAAACCCGCTGATCAGCCTCGACTGTGCCTT
CTAGTTGCCAGCCATCTGTTGTTTGCCCCTCCCCCGTGCCTTCCTTGACCCTGGAAGG
TGCCACTCCCACTGTCCTTTCCTAATAAAATGAGGAAATTGCATCGCATTGTCTGAG
TAGGTGTCATTCTATTCTGGGGGGTGGGGTGGGGCAGGACAGCAAGGGGGAGGATT
GGGAAGACAATAGCAGGCATGCTGGGGATGCGGTGGGCTCTATGGGTCTCGACATT
GATTATTGACTAGTTATTAATAGTAATCAATTACGGGGTCATTAGTTCATAGCCCAT
ATATGGAGTTCCGCGTTACATAACTTACGGTAAATGGCCCGCCTGGCTGACCGCCCA
ACGACCCCCGCCCATTGACGTCAATAATGACGTATGTTCCCATAGTAACGCCAATAG
GGACTTTCCATTGACGTCAATGGGTGGAGTATTTACGGTAAACTGCCCACTTGGCAG
TACATCAAGTGTATCATATGCCAAGTACGCCCCCTATTGACGTCAATGACGGTAAAT
GGCCCGCCTGGCATTATGCCCAGTACATGACCTTATGGGACTTTCCTACTTGGCAGT
ACATCTACGTATTAGTCATCGCTATTACCATGGTCGAGGTGAGCCCCACGTTCTGCT
TCACTCTCCCCATCTCCCCCCCCTCCCCACCCCCAATTTTGTATTTATTTATTTTTTAA
TTATTTTGTGCAGCGATGGGGGCGGGGGGGGGGGGGGGCGAGGCGGAGAGGTGCG
GCGGCAGCCAATCAGAGCGGCGCGCTCCGAAAGTTTCCTTTTATGGCGAGGCGGCG
GCGGCGGCGGCCCTATAAAAAGCGAAGCGCGCGGCGGGCGGGAGTCGCTGCGCGC
TGCCTTCGCCCCGTGCCCCGCTCCGCCGCCGCCTCGCGCCGCCCGCCCCGGCTCTGA
CTGACCGCGTTACTCCCACAGGTGAGCGGGCGGGACGGCCCTTCTCCTCCGGGCTGT
AATTAGCGCTTGGTTTAATGACGGCTTGTTTCTTTTCTGTGGCTGCGTGAAAGCCTTG
AGGGGCTCCGGGAGGGCCCTTTGTGCGGGGGGAGCGGCTCGGGGGGTGCGTGCGTG
TGTGTGTGCGTGGGGAGCGCCGCGTGCGGCTCCGCGCTGCCCGGCGGCTGTGAGCG
CTGCGGGCGCGGCGCGGGGCTTTGTGCGCTCCGCAGTGTGCGCGAGGGGAGCGCGG
CCGGGGGCGGTGCCCCGCGGTGCGGGGGGGGCTGCGAGGGGAACAAAGGCTGCGT
GCGGGGTGTGTGCGTGGGGGGGTGAGCAGGGGGTGTGGGCGCGTCGGTCGGGCTGC
AACCCCCCCTGCACCCCCCTCCCCGAGTTGCTGAGCACGGCCCGGCTTCGGGTGCGG
GGCTCCGTACGGGGCGTGGCGCGGGGCTCGCCGTGCCGGGCGGGGGGTGGCGGCAG
GTGGGGGTGCCGGGCGGGGGGGGGCCGCCTCGGGCCGGGGAGGGCTCGGGGGAGG
GGCGCGGCGGCCCCCGGAGCGCCGGCGGCTGTCGAGGCGCGGCGAGCCGCAGCCAT
TGCCTTTTATGGTAATCGTGCGAGAGGGCGCAGGGACTTCCTTTGTCCCAAATCTGT
GCGGAGCCGAAATCTGGGAGGCGCCGCCGCACCCCCTCTAGCGGGCGCGGGGCGAA
GCGGTGCGGCGCCGGCAGGAAGGAAATGGGCGGGGAGGGCCTTCGTGCGTCGCCG
CGCCGCCGTCCCCTTCTCCCTCTCCAGCCTCGGGGCTGTCCGCGGGGGGACGGCTGC
CTTCGGGGGGGACGGGGCAGGGCGGGGTTCGGCTTCTGGCGTGTGACCGGCGGCTC
TAGAGCCTCTGCTAACCATGTTCATGCCTTCTTCTTTTTCCTACAGCTCCTGGGCAAC
GTGCTGGTTATTGTGCTGTCTCATCATTTTGGCAAAGAATTCGTCGACATGTCTCGCT
CCGTGGCCTTAGCTGTGCTCGCGCTACTCTCTCTTTCTGGCCTCGAGGCTAAAGGAC
CTCCTGCCGCTCTTACACTTGGTGGTGGCGGATCCGGTGGTGGCGGTTCTGGTGGTG
GCGGCTCCATCCAGCGTACGCCAAAGATTCAGGTTTACTCACGTCATCCAGCAGAG
AATGGAAAGTCAAATTTCCTGAATTGCTATGTGTCTGGGTTTCATCCATCCGACATT
GAAGTTGACTTACTGAAGAATGGAGAGAGAATTGAAAAAGTGGAGCATTCAGACTT
GTCTTTCAGCAAGGACTGGTCTTTCTATCTCTTGTACTACACTGAGTTCACCCCCACT
GAAAAAGATGAGTATGCCTGCCGTGTGAACCATGTGACTTTGTCACAGCCCAAGAT
AGTTAAGTGGGATCGCGACATGGGTGGTGGCGGTTCTGGTGGTGGCGGTAGTGGCG
GCGGAGGAAGCGGTGGTGGCGGTTCCGGCTCCCACTCCATGAGGTATTTCAGCGCC
GCCGTGTCCCGGCCCGGCCGCGGGGAGCCCCGCTTCATCGCCATGGGCTACGTGGA
CGACACGCAGTTCGTGCGGTTCGACAGCGACTCGGCGTGTCCGAGGATGGAGCCGC
GGGCGCCGTGGGTGGAGCAGGAGGGGCCGGAGTATTGGGAAGAGGAGACACGGAA
CACCAAGGCCCACGCACAGACTGACAGAATGAACCTGCAGACCCTGCGCGGCTACT
ACAACCAGAGCGAGGCCAGTTCTCACACCCTCCAGTGGATGATTGGCTGCGACCTG
GGGTCCGACGGACGCCTCCTCCGCGGGTATGAACAGTATGCCTACGATGGCAAGGA
TTACCTCGCCCTGAACGAGGACCTGCGCTCCTGGACCGCAGCGGACACTGCGGCTC
AGATCTCCAAGCGCAAGTGTGAGGCGGCCAATGTGGCTGAACAAAGGAGAGCCTAC
CTGGAGGGCACGTGCGTGGAGTGGCTCCACAGATACCTGGAGAACGGGAAGGAGA
TGCTGCAGCGCGCGGACCCCCCCAAGACACACGTGACCCACCACCCTGTCTTTGACT
ATGAGGCCACCCTGAGGTGCTGGGCCCTGGGCTTCTACCCTGCGGAGATCATACTGA
CCTGGCAGCGGGATGGGGAGGACCAGACCCAGGACGTGGAGCTCGTGGAGACCAG
GCCTGCAGGGGATGGAACCTTCCAGAAGTGGGCAGCTGTGGTGGTGCCTTCTGGAG
AGGAGCAGAGATACACGTGCCATGTGCAGCATGAGGGGCTGCCGGAGCCCCTCATG
CTGAGATGGAAGCAGTCTTCCCTGCCCACCATCCCCATCATGGGTATCGTTGCTGGC
CTGGTTGTCCTTGCAGCTGTAGTCACTGGAGCTGCGGTCGCTGCTGTGCTGTGGAGA
AAGAAGAGCTCAGATTGAACGCGTGAATTCGATATCAAGCTTATCGATAATCAACC
TCTGGATTACAAAATTTGTGAAAGATTGACTGGTATTCTTAACTATGTTGCTCCTTTT
ACGCTATGTGGATACGCTGCTTTAATGCCTTTGTATCATGCTATTGCTTCCCGTATGG
CTTTCATTTTCTCCTCCTTGTATAAATCCTGGTTGCTGTCTCTTTATGAGGAGTTGTG
GCCCGTTGTCAGGCAACGTGGCGTGGTGTGCACTGTGTTTGCTGACGCAACCCCCAC
TGGTTGGGGCATTGCCACCACCTGTCAGCTCCTTTCCGGGACTTTCGCTTTCCCCCTC
CCTATTGCCACGGCGGAACTCATCGCCGCCTGCCTTGCCCGCTGCTGGACAGGGGCT
CGGCTGTTGGGCACTGACAATTCCGTGGTGTTGTCGGGGAAATCATCGTCCTTTCCT
TGGCTGCTCGCCTATGTTGCCACCTGGATTCTGCGCGGGACGTCCTTCTGCTACGTCC
CTTCGGCCCTCAATCCAGCGGACCTTCCTTCCCGCGGCCTGCTGCCGGCTCTGCGGC
CTCTTCCGCGTCTTCGCCTTCGCCCTCAGACGAGTCGGATCTCCCTTTGGGCCGCCTC
CCCGCATCGATACCGAGCGCTGCTCGAGAGATCTACGGGTGGCATCCCTGTGACCCC
TCCCCAGTGCCTCTCCTGGCCCTGGAAGTTGCCACTCCAGTGCCCACCAGCCTTGTC
CTAATAAAATTAAGTTGCATCATTTTGTCTGACTAGGTGTCCTTCTATAATATTATGG
GGTGGAGGGGGGTGGTATGGAGCAAGGGGCAAGTTGGGAAGACAACCTGTAGGGC
CTGCGGGGTCTATTGGGAACCAAGCTGGAGTGCAGTGGCACAATCTTGGCTCACTG
CAATCTCCGCCTCCTGGGTTCAAGCGATTCTCCTGCCTCAGCCTCCCGAGTTGTTGG
GATTCCAGGCATGCATGACCAGGCTCAGCTAATTTTTGTTTTTTTGGTAGAGACGGG
GTTTCACCATATTGGCCAGGCTGGTCTCCAACTCCTAATCTCAGGTGATCTACCCAC
CTTGGCCTCCCAAATTGCTGGGATTACAGGCGTGAACCACTGCTCCCTTCCCTGTCC
TTCTGATTTTGTAGGTAACCAGGCGCGTGAATTCACTCCTCAGGTGCAGGCTGCCTA
TCAGAAGGTGGTGGCTGGTGTGGCCAATGCCCTGGCTCACAAATACCACTGAGATC
TTTTTCCCTCTGCCAAAAATTATGGGGACATCATGAAGCCCCTTGAGCATCTGACTT
CTGGCTAATAAAGGAAATTTATTTTCATTGCAATAGTGTGTTGGAATTTTTTGTGTCT
CTCACTCGGAAGGACATATGGGAGGGCAAATCATTTAAAACATCAGAATGAGTATT
TGGTTTAGAGTTTGGCAACATATGCCCATATGCTGGCTGCCATGAACAAAGGTTGGC
TATAAAGAGGTCATCAGTATATGAAACAGCCCCCTGCTGTCCATTCCTTATTCCATA
GAAAAGCCTTGACTTGAGGTTAGATTTTTTTTATATTTTGTTTTGTGTTATTTTTTTCT
TTAACATCCCTAAAATTTTCCTTACATGTTTTACTAGCCAGATTTTTCCTCCTCTCCTG
ACTACTCCCAGTCATAGCTGTCCCTCTTCTCTTATGGAGATCCCTCGACCTGCAGCCC
AAGCTTGGCGTAATCATGGTCATAGCTGTTTCCTGTGTGAAATTGTTATCCGCTCAC
AATTCCACACAACATACGAGCCGGAAGCATAAAGTGTAAAGCCTGGGGTGCCTAAT
GAGTGAGCTAACTCACATTAATTGCGTTGCGCTCACTGCCCGCTTTCCAGTCGGGAA
ACCTGTCGTGCCAGCGGATCGACAGTACTAAGCTTTACTAGGGACAGGATTGGTGA
CAGAAAAGCCCCATCCTTAGGCCTCCTCCTTCCTAGTCTCCTGATATTGGGTCTAAC
CCCCACCTCCTGTTAGGCAGATTCCTTATCTGGTGACACACCCCCATTTCCTGGAGC
CATCTCTCTCCTTGCCAGAACCTCTAAGGTTTGCTTACGATGGAGCCAGAGAGGATC
CTGGGAGGGAGAGCTTGGCAGGGGGTGGGAGGGAAGGGGGGGATGCGTGACCTGC
CCGGTTCTCAGTGGCCACCCTGCGCTACCCTCTCCCAGAACCTGAGCTGCTCTGACG
CGGCTGTCTGGTGCGTTTCACTGATCCTGGTGCTGCAGCTTCCTTACACTTCCCAAGA
GGAGAAGCAGTTTGGAAAAACAAAATCAGAATAAGTTGGTCCTGAGTTCTAACTTT
GGCTCTTCACCTTTCTAGTCCCCAATTTATATTGTTCCTCCGTGCGTCAGTTTTACCT
GTGAGATAAGGCCAGTAGCCAGCCCCGTCCTGGCAGGGCTGTGGTGAGGAGGGGGG
TGTCCGTGTGGAAAACTCCCTTTGTGAGAATGGTGCGTCCTAGGTGTTCACCAGGTC
GTGGCCGCCTCTACTCCCTTTCTCTTTCTCCATCCTTCTTTCCTTAAAGAGTCCCCAGT
GCTATCTGGGACATATTCCTCCGCCCAGAGCAGGGTCCCGCTTCCCTAAGGCCCTGC
TCTGGGCTTCTGGGTTTGAGTCCTTGGCAAGCCCAGGAGAGGCGCTCAGGCTTCCCT
GTCCCCCTTCCTCGTCCACCATCTCATGCCCCTGGCTCTCCTGCCCCTTCCCTACAGG
GGTTCCTGGCTCTGCTCTAAGGGCGAATTCGCGGCCGCTAAATTCAATTCGCCCTAT
AGTGAGTCGTATTACAATTCACTGGCCGTCGTTTTACAACGTCGTGACTGGGAAAAC
CCTGGCGTTACCCAACTTAATCGCCTTGCAGCACATCCCCCTTTCGCCAGCTGGCGT
AATAGCGAAGAGGCCCGCACCGATCGCCCTTCCCAACAGTTGCGCAGCCTATACGT
ACGGCAGTTTAAGGTTTACACCTATAAAAGAGAGAGCCGTTATCGTCTGTTTGTGGA
TGTACAGAGTGATATTATTGACACGCCGGGGCGACGGATGGTGATCCCCCTGGCCA
GTGCACGTCTGCTGTCAGATAAAGTCTCCCGTGAACTTTACCCGGTGGTGCATATCG
GGGATGAAAGCTGGCGCATGATGACCACCGATATGGCCAGTGTGCCGGTCTCCGTT
ATCGGGGAAGAAGTGGCTGATCTCAGCCACCGCGAAAATGACATCAAAAACGCCAT
TAACCTGATGTTCTGGGGAATATAAATGTCAGGCATGAGATTATCAAAAAGGATCTT
CACCTAGATCCTTTTCACGTAGAAAGCCAGTCCGCAGAAACGGTGCTGACCCCGGA
TGAATGTCAGCTACTGGGCTATCTGGACAAGGGAAAACGCAAGCGCAAAGAGAAA
GCAGGTAGCTTGCAGTGGGCTTACATGGCGATAGCTAGACTGGGCGGTTTTATGGA
CAGCAAGCGAACCGGAATTGCCAGCTGGGGCGCCCTCTGGTAAGGTTGGGAAGCCC
TGCAAAGTAAACTGGATGGCTTTCTTGCCGCCAAGGATCTGATGGCGCAGGGGATC
AAGCTCTGATCAAGAGACAGGATGAGGATCGTTTCGCATGATTGAACAAGATGGAT
TGCACGCAGGTTCTCCGGCCGCTTGGGTGGAGAGGCTATTCGGCTATGACTGGGCAC
AACAGACAATCGGCTGCTCTGATGCCGCCGTGTTCCGGCTGTCAGCGCAGGGGCGC
CCGGTTCTTTTTGTCAAGACCGACCTGTCCGGTGCCCTGAATGAACTGCAAGACGAG
GCAGCGCGGCTATCGTGGCTGGCCACGACGGGCGTTCCTTGCGCAGCTGTGCTCGAC
GTTGTCACTGAAGCGGGAAGGGACTGGCTGCTATTGGGCGAAGTGCCGGGGCAGGA
TCTCCTGTCATCTCACCTTGCTCCTGCCGAGAAAGTATCCATCATGGCTGATGCAAT
GCGGCGGCTGCATACGCTTGATCCGGCTACCTGCCCATTCGACCACCAAGCGAAAC
ATCGCATCGAGCGAGCACGTACTCGGATGGAAGCCGGTCTTGTCGATCAGGATGAT
CTGGACGAAGAGCATCAGGGGCTCGCGCCAGCCGAACTGTTCGCCAGGCTCAAGGC
GAGCATGCCCGACGGCGAGGATCTCGTCGTGACCCATGCATGGCGATGCCTGCTTG
CCGAATATCATGGTGGAAAATGGCCGCTTTTCTGGATTCATCGACTGTGGCCGGCTG
GGTGTGGCGGACCGCTATCAGGACATAGCGTTGGCTACCCGTGATATTGCTGAAGA
GCTTGGCGGCGAATGGGCTGACCGCTTCCTCGTGCTTTACGGTATCGCCGCTCCCGA
TTCGCAGCGCATCGCCTTCTATCGCCTTCTTGACGAGTTCTTCTGAATTATTAACGCT
TACAATTTCCTGATGCGGTATTTTCTCCTTACGCATCTGTGCGGTATTTCACACCGCA
TCAGGTGGCACTTTTCGGGGAAATGTGCGCGGAACCCCTATTTGTTTATTTTTCTAA
ATACATTCAAATATGTATCCGCTCATGAGATTATCAAAAAGGATCTTCACCTAGATC
CTTTTAAATTAAAAATGAAGTTTTAAATCAATCTAAAGTATATATGAGTAAACTTGG
TCTGACAGTTACCAATGCTTAATCAGTGAGGCACCTATCTCAGCGATCTGTCTATTT
CGTTCATCCATAGTTGCCTGACTCCCCGTCGTGTAGATAACTACGATACGGGAGGGC
TTACCATCTGGCCCCAGTGCTGCAATGATACCGCGAGACCCACGCTCACCGGCTCCA
GATTTATCAGCAATAAACCAGCCAGCCGGAAGGGCCGAGCGCAGAAGTGGTCCTGC
AACTTTATCCGCCTCCATCCAGTCTATTAATTGTTGCCGGGAAGCTAGAGTAAGTAG
TTCGCCAGTTAATAGTTTGCGCAACGTTGTTGCCATTGCTACAGGCATCGTGGTGTC
ACGCTCGTCGTTTGGTATGGCTTCATTCAGCTCCGGTTCCCAACGATCAAGGCGAGT
TACATGATCCCCCATGTTGTGCAAAAAAGCGGTTAGCTCCTTCGGTCCTCCGATCGT
TGTCAGAAGTAAGTTGGCCGCAGTGTTATCACTCATGGTTATGGCAGCACTGCATAA
TTCTCTTACTGTCATGCCATCCGTAAGATGCTTTTCTGTGACTGGTGAATACTCAACC
AAGTCATTCTGAGAATAGTGTATGCGGCGACCGAGTTGCTCTTGCCCGGCGTCAATA
CGGGATAATACCGCGCCACATAGCAGAACTTTAAAAGTGCTCATCATTGGAAAACG
TTCTTCGGGGCGAAAACTCTCAAGGATCTTACCGCTGTTGAGATCCAGTTCGATGTA
ACCCACTCGTGCACCCAACTGATCTTCAGCATCTTTTACTTTCACCAGCGTTTCTGGG
TGAGCAAAAACAGGAAGGCAAAATGCCGCAAAAAAGGGAATAAGGGCGACACGGA
AATGTTGAATACTCATACTCTTCCTTTTTCAATATTATTGAAGCATTTATCAGGGTTA
TTGTCTCATGACCAAAATCCCTTAACGTGAGTTTTCGTTCCACTGAGCGTCAGACCC
CGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGC
TACCAACTCTTTTTCCGAAGGTAACTGGCTTCAGCAGAGCGCAGATACCAAATACTG
TTCTTCTAGTGTAGCCGTAGTTAGGCCACCACTTCAAGAACTCTGTAGCACCGCCTA
CATACCTCGCTCTGCTAATCCTGTTACCAGTGGCTGCTGCCAGTGGCGATAAGTCGT
GTCTTACCGGGTTGGACTCAAGACGATAGTTACCGGATAAGGCGCAGCGGTCGGGC
TGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAG
GCGGACAGGTATCCGGTAAGCGGCAGGGTCGGAACAGGAGAGCGCACGAGGGAGC
TTCCAGGGGGAAACGCCTGGTATCTTTATAGTCCTGTCGGGTTTCGCCACCTCTGAC
TTGAGCGTCGATTTTTGTGATGCTCGTCAGGGGGGCGGAGCCTATGGAAAAACGCC
AGCAACGCGGCCTTTTTACGGTTCCTGGCCTTTTGCTGGCCTTTTGCTCACATGTTCT
TTCCTGCGTTATCCCCTGATTCTGTGGATAACCGTATTACCGCCTTTGAGTGAGCTGA
TACCGCTCGCCGCAGCCGAACGACCGAGCGCAGCGAGTCAGTGAGCGAGGAAGCG
GAAGAGCGCCCAATACGCAAACCGCCTCTCCCCGCGCGTTGGCCGATTCATTAATGC
AGCTGGCACGACAGGTTTCCCGACTGGAAAGCGGGCAGTGAGCGCAACGCAATTAA
TGTGAGTTAGCTCACTCATTAGGCACCCCAGGCTTTACACTTTATGCTTCCGGCTCGT
ATGTTGTGTGGAATTGTGAGCGGATAACAATTTCACACAGGAAACAGCTATGACCA
TGATTACGCCAAGCTCAGAATTAACCCTCACTAAAGGGACTAGCT
(P53DD sequence)
SEQ ID NO: 36
ATGACTGCCATGGAGGAGTCACAGTCGGATATCAGCCTCAAGAGAGCGCTGCCCAC
CTGCACAAGCGCCTCTCCCCCGCAAAAGAAAAAACCACTTGATGGAGAGTATTTCA
CCCTCAAGATCCGCGGGCGTAAACGCTTCGAGATGTTCCGGGAGCTGAATGAGGCC
TTAGAGTTAAAGGATGCCCATGCTACAGAGGAGTCTGGAGACAGCAGGGCTCACTC
CAGCTACCTGAAGACCAAGAAGGGCCAGTCTACTTCCCGCCATAAAAAAACAATGG
TCAAGAAAGTGGGGCCTGACTCAGACTGA
(sgRNA 1)
SEQ ID NO: 37
CGCGAGCACAGCTAAGGCCA
(sgRNA 2)
SEQ ID NO: 38
TGTGAACCATGTGACTTTGTC
SEQ ID NO: 39
ATGTCTCGCTCCGTGGCCTTAGCTGTGCTCGCGCTACTCTCT
SEQ ID NO: 40
AGCTGTGCTCGCGCTACTCTCT
SEQ ID NO: 41
ATGTCTCGCTCCGTGGCTTAGCTGTGCTCGCGCTACTCTCT
SEQ ID NO: 42
ATGTCTCGCTCCGTGGGCTCGCGCTACTCTCT

1. A recombinant polynucleotide comprising
a first polynucleotide sequence encoding a beta-2-Microglobulin (B2M) signaling peptide or a fragment thereof;
a second polynucleotide sequence encoding a nonamer or a fragment thereof;
a third polynucleotide sequence encoding a B2M polypeptide that lacks signaling peptide; and
a fourth polynucleotide sequence encoding a human leukocyte antigen (HLA)-E polypeptide or an HLA-G polypeptide or a fragment thereof.

2. (canceled)

3. The recombinant polynucleotide of claim 1, wherein the fourth polynucleotide sequence comprises a sequence at least about 80% identity to SEQ ID NO: 20, SEQ ID NO: 14, SEQ ID NO: 11, or a fragment thereof.

4-6. (canceled)

7. The recombinant polynucleotide of claim 1, wherein the nonamer comprises a signal peptide sequence of a class I HLA, wherein the class I HLA is HLA-C.

8. (canceled)

9. The recombinant polynucleotide of claim 1, wherein the second polynucleotide sequence is at least about 80% identity to SEQ ID NO: 5 or a fragment thereof.

10. The recombinant of claim 1, wherein the first polynucleotide is at least about 80% identity to SEQ ID NO: 1 or 16 or a fragment thereof.

11. The recombinant of claim 1, wherein the third polynucleotide is at least about 80% identity to SEQ ID NO: 3 or 18 or a fragment thereof.

12. The recombinant polynucleotide of claim 1, wherein the recombinant polynucleotide comprises a sequence at least 80% identity to SEQ ID NO: 13, SEQ ID NO: 22, or a fragment thereof.

13. A vector comprising the recombinant polynucleotide of claim 1.

14. (canceled)

15. (canceled)

16. A universal stem cell comprising the recombinant polynucleotide of claim 1.

17. The universal stem cell of claim 16, further comprising a polynucleotide encoding a Cas9 and a polynucleotide encoding a guide RNA; wherein the guide RNA targets the beta-2-Microglobulin gene or a fragment thereof.

18-20. (canceled)

21. The universal stem cell of claim 16, further comprising a polynucleotide encoding a p53 dominant-negative (p53 DD) polypeptide.

22. (canceled)

23. The universal stem cell of claim 16, wherein the universal stem comprising a deletion in B2M host gene or a fragment thereof.

24. A method of making a universal stem cell, comprising
transducing a recombinant polynucleotide into the universal stem cell, and
culturing the transduced universal stem cell,
wherein the recombinant polynucleotide comprises
a first polynucleotide sequence encoding a beta-2-Microglobulin (B2M) signaling peptide or a fragment thereof;
a second polynucleotide sequence encoding a nonamer or a fragment thereof;
a third polynucleotide sequence encoding a B2M polypeptide that lacks signaling peptide; and
a fourth polynucleotide sequence encoding a HLA-E or HLA-G polypeptide or a fragment thereof.

25. (canceled)

26 The method of claim 25, wherein the fourth polynucleotide sequence comprises a sequence at least about 80% identity to SEQ ID NO: 20, SEQ ID NO: 14, SEQ ID NO:: 11, or a fragment thereof.

27-29. (canceled)

30. The method of claim 24, wherein the nonamer comprises a signal peptide sequence of a class I HLA; wherein the class I HLA is HLA-C.

31. (canceled).

32. The method of claim 24, wherein the second polynucleotide sequence is at least about 80% identity to SEQ ID NO: 5 or a fragment thereof.

33. The method of claim 24, wherein first nucleotide is at least about 80% identity to SEQ ID NO: 1 or 16 or a fragment thereof.

34. The method of claim 24, wherein the third nucleotide is at least about 80% identity to SEQ ID NO: 3 or 18 or a fragment thereof.

35. The method of claim 24, comprises a sequence at least about 80% identity to SEQ ID NO: 13, SEQ ID NO: 22, or a fragment thereof.

36-38. (canceled)

39. The method of claim 24, further comprising transducing the universal stem cell a polynucleotide encoding a Cas9 and a polynucleotide encoding a guide RNA; wherein the guide RNA targets the beta-2-Microglobulin gene or a fragment thereof.

40. (canceled)

41. (canceled)

42. The method of claim 24, further comprising transducing the universal stem cell a polynucleotide encoding a p53 dominant-negative (p53 DD) polypeptide (p53 DD).

43. (canceled)

44. A method of making a cardiomyocyte, comprising making a universal stem cell by the method of claim 24;
culturing the universal stem cell for at least about 7 days in a cell culture media to differentiate the universal stem cell into a cardiomyocyte; and
harvesting the differentiated cardiomyocyte.

45. A method for treating a cardiac disorder in a subject in need, comprising
making a universal stem cell by the method of claim 24;
culturing the universal stem cell for at least about 7 days in a cell culture media to differentiate the universal stem cell into a cardiomyocyte; and
transplanting the cardiomyocyte into the subject.

46-48. (canceled)

49. The method of claim 45, wherein the universal stem cell is cultured for at least about 12 days to differentiate the universal stem cell into a cardiomyocyte.
